(19) United States
(12) Patent Application Publication
(10) Pub. No.: US 2017/0151362 A1
(43) Pub. Date: Jun. 1, 2017

(54) TABLE TOP SCENT DISPENSING DEVICE AND METHOD

(71) Applicant: Vapor Communications, Inc., Cambridge, MA (US)

(72) Inventors: David A. Edwards, Cambridge, MA (US); Rachel Diane Field, Huntington Beach, CA (US)

(21) Appl. No.: 15/029,544

(22) PCT Filed: Oct. 15, 2014
(86) PCT No.: PCT/US2014/060630

§ 371 Date: Apr. 14, 2016

Related U.S. Application Data
(60) Provisional application No. 61/891,328, filed on Oct. 15, 2013.
(60) Provisional application No. 61/944,862, filed on Feb. 26, 2014.

(51) Int. Cl. A61L 9/12; B05B 7/24; B65D 25/04

(52) U.S. Cl. CPC A61L 9/122; B65D 25/04; B05B 7/2472; A61L 2209/133

(57) ABSTRACT

A method of operating a scent dispensing device includes starting operation of an air mover to move air. A motor orients a scent cartridge in a first orientation in which an outlet of a first chamber of the scent cartridge is fluidly communicatively coupled with a port of the scent dispensing device. A direction and an amount of movement from the first orientation to orient the scent cartridge in a second orientation in which an outlet of a second chamber of the scent cartridge is fluidly communicatively coupled with the first port of the scent dispensing device are determined. The scent cartridge is then oriented in the second orientation. A consumable scent cartridge includes a housing having walls that define chambers arrayed about a rotational axis and consumable scent media received in at least two of the chambers.

### BACKGROUND

Technical Field

The present disclosure relates generally to the selective dispensing of combinations and sequences scents that are specified in scent specifications.

Description of the Related Art

The sense of smell (i.e., olfactory perception) is extremely important to humans. For example, the smell of smoke can alert a person of the potential dangers of a nearby fire. Additionally, smells can trigger memories and impact our emotions. Various forms of aromatherapy have been investigated in attempts to alter a person's mind, mood, cognitive function, and/or health.

A number of systems and devices have been developed for selectively providing olfactory sensations. For a variety of reasons, such systems and devices have proven to be inadequate and have not gained commercial acceptance. For example, the Smell-O-Vision system was developed over fifty years ago, which caused a number of different odors to be emitted from the seats in a movie theater when triggered by a film's soundtrack. That system was expensive and had technical problems. Accordingly, the Smell-O-Vision system was used in conjunction with only one film.

New approaches that enable users to quickly and easily generate customized scent specifications for communicating specific themes, ideas, and/or feelings using sequences and combinations of scents are desirable. Additionally, new systems and devices that dispense scents based on such scent specifications are desirable.

### BRIEF SUMMARY

A method of operating a scent dispensing device may be summarized as including starting operation of an air mover to move air; following the starting operation of the air mover, orienting via a first motor a first scent cartridge in a first orientation in which a respective outlet of a first one of a plurality of chambers of the first scent cartridge is fluidly communicatively coupled with a first port of the scent dispensing device; determining via at least one controller a direction of movement and an amount of movement in the determined direction of movement from the first orientation to orient the first scent cartridge in a second orientation in which a respective outlet of a second one of the plurality of chambers is fluidly communicatively coupled with the first port of the scent dispensing device; and orienting via the first motor the first scent cartridge in the second orientation in which the respective outlet of the second one of the plurality of chambers is fluidly communicatively coupled with the first port of the scent dispensing device.

Determining a direction of movement may include determining which of a first rotational direction and a second rotational direction provides a least amount of movement to move the first scent cartridge from the first orientation to the second orientation, the second rotational direction opposite the first rotational direction. Determining an amount of movement in the determined direction of movement may include determining a number of steps by a stepper motor in the first rotational direction that will orient the first scent cartridge in the second orientation from the first orientation. Determining an amount of movement in the determined direction of movement may include determining a number of steps by a stepper motor in the first rotational direction that will orient the first scent cartridge in the second orientation from the first orientation. Orienting the first scent cartridge in the second orientation may include moving the first scent cartridge from the first orientation in the determined direction of movement by the determined amount of movement. The first direction may be a first rotational direction, and orienting the first scent cartridge in the second orientation may include driving a stepper motor to move the first scent cartridge in the first rotational direction by a determined first number of steps of the stepper motor. The method may further include in response to a first indication, orienting via the first motor the first scent cartridge in a default orientation. Orienting the first scent cartridge in the default orientation may include orienting the first scent cartridge such that none of the plurality of chambers which contain scent media are fluidly communicatively coupled with the first port of the scent dispensing device. Orienting the first scent cartridge in the default orientation may include orienting the first scent cartridge such that a third one of the plurality of chambers which is empty is fluidly communicatively coupled with the first port of the scent dispensing device. Orienting the first scent cartridge in the default orientation may include orienting the first scent cartridge such that a third one of the plurality of chambers in which an air filter is located is fluidly communicatively coupled with the first port of the scent dispensing device. The method may further include detecting a low available power condition of a power source via the at least one controller, and providing the first indication in response to the detection of the low available power condition. The method may further include detecting a turn off input via the at least one controller, and providing the first indication in response to the detection of the turn off input. The method may further include stopping the operation of the air mover after orienting the first scent cartridge in the default orientation in response to the first indication. The method may further include continuously operating the air mover to move air between the starting operation of the air mover and the stopping of the operation of the air mover in response to the first indication. The method may further include determining a direction of movement and an amount of movement in the determined direction of movement from the second orientation to orient a third one of the plurality of chambers of the first scent cartridge with the first port of the scent dispensing device via the at least one controller; and orienting via the first motor the first scent cartridge in a third orientation in which a respective outlet of the second one of the plurality of chambers is fluidly communicatively coupled with the first port of the scent dispensing device; and continuously operating the air mover to move air between the starting operation of the air mover and the stopping of the operation of the air mover in response to the first indication. The method may further include orienting via the first motor the first scent cartridge in the default orientation between orienting the first scent cartridge in the second orientation and orienting the first scent cartridge in a third orientation. The method may further include determining a sequence of orientations of the first scent cartridge based at least in part on a scent specification via the at least one controller, the sequence of orientations including at least the first and the second orientations. The method may further include determining via the at least one controller a direction of movement and an amount of movement in the determined direction of movement from the default orientation to orient the first scent cartridge in the first orientation; and orienting via the first motor the first scent cartridge in the first orientation in which the respective outlet of the first one of the plurality of chambers is fluidly communicatively coupled with the first port of the scent dispensing device before orienting the first scent cartridge in the second orientation. The method may further include wirelessly receiving the scent specification via a radio prior to determining the sequence of orientations of the first scent cartridge.

A scent dispensing device may be summarized as including an air mover that moves air; a first motor that orients a first scent cartridge in a first orientation in which a respective outlet of a first one of a plurality of chambers of the first scent cartridge is fluidly communicatively coupled with a first port of the scent dispensing device after operation of the air mover has been started; and at least one controller that determines a direction of movement and an amount of movement in the determined direction of movement from the first orientation to orient the first scent cartridge in a second orientation in which a respective outlet of a second one of the plurality of chambers is fluidly communicatively coupled with the first port of the scent dispensing device, and that causes the first motor to orient the first scent cartridge in the second orientation in which the respective outlet of the second one of the plurality of chambers is fluidly communicatively coupled with the first port of the scent dispensing device.

The at least one controller may determine which of a first rotational direction and a second rotational direction provides a least amount of movement to move the first scent cartridge from the first orientation to the second orientation, the second rotational direction opposite the first rotational direction. The at least one controller may determine a number of steps by a stepper motor in the first rotational direction that will orient the first scent cartridge in the second orientation from the first orientation. The at least one controller may determine a number of steps by a stepper motor in the first rotational direction that will orient the first scent cartridge in the second orientation from the first orientation. The at least one controller may cause the first motor to move the first scent cartridge from the first orientation in the determined direction of movement by the determined amount of movement. The first direction may be a first rotational direction, the first motor may be a stepper motor, and the at least one controller may cause the first motor to move the first scent cartridge in the first rotational direction by a determined first number of steps. The at least one controller may cause the first motor to orient the first scent cartridge in a default orientation in response to a first indication. The at least one controller may cause the first motor to orient the first scent cartridge in the default orientation such that none of the plurality of chambers which contain scent media are fluidly communicatively coupled with the first port of the scent dispensing device. The at least one controller may cause the first motor to orient the first scent cartridge in the default orientation such that a third one of the plurality of chambers which is empty is fluidly communicatively coupled with the first port of the scent dispensing device. The at least one controller may cause the first motor to orient the first scent cartridge in the default orientation such that a third one of the plurality of chambers in which an air filter is located is fluidly communicatively coupled with the first port of the scent dispensing device. The at least one controller may detect a low available power condition of a power source and may provide the first indication in response to a detection of the low available power condition. The at least one controller may detect a turn off input and may provide the first indication in response to a detection of the turn off input. The at least one controller may stop the operation of the air mover after orienting the first scent cartridge in the default orientation in response to the first indication. The at least one controller may cause the air mover to continuously operate to move air between starting operation of the air mover and stopping operation of the air mover in response to the first indication. The at least one controller: may determine a direction of movement and an amount of movement in the determined direction of movement from the second orientation to orient a third one of the plurality of chambers of the first scent cartridge with the first port of the scent dispensing device via the at least one controller, and may cause the first motor to orient the first scent cartridge in a third orientation in which a respective outlet of the second one of the plurality of chambers is fluidly communicatively coupled with the first port of the scent dispensing device, and may cause the air mover to continuously operate to move air between starting operation of the air mover and stopping operation of the air mover in response to the first indication. The at least one controller may cause the first motor to orient the first scent cartridge in the default orientation between orienting the first scent cartridge in the second orientation and orienting the first scent cartridge in a third orientation. The at least one controller may determine a sequence of orientations of the first scent cartridge based at least in part on a scent specification, the sequence of orientations including at least the first and the second orientations. The at least one controller: may determine a direction of movement and an amount of movement in the determined direction of movement from the default orientation to orient the first scent cartridge in the first orientation; and may cause the first motor to orient the first scent cartridge in the first orientation in which the respective outlet of the first one of the plurality of chambers is fluidly communicatively coupled with the first port of the scent dispensing device before orienting the first scent cartridge in the second orientation. The device may further include a communications module that wirelessly receives the scent specification via a radio, wherein the communications module receives the scent specification before the at least one controller determines the sequence of orientations of the first scent cartridge.

A method of operating a scent dispensing device may be summarized as including receiving a first scent cartridge having a plurality of chambers at least some of the chambers having respective scent media therein; with the first scent cartridge in a default orientation, starting operation of an air mover to move air with respect to the first scent cartridge; following the starting operation of the air mover, orienting via a first motor the first scent cartridge from the default orientation to a first orientation in which a respective outlet of a first one of the plurality of chambers of the first scent cartridge is fluidly communicatively coupled with a first port of the scent dispensing device; and in response to an indication of a stop condition, returning the first scent cartridge to the default orientation.

Returning the first scent cartridge to the default orientation may include orienting the first scent cartridge such that none of the plurality of chambers which contain scent media are fluidly communicatively coupled with the first port of the scent dispensing device. Returning the first scent cartridge to the default orientation may include orienting the first scent cartridge such that one of the plurality of chambers which is empty is fluidly communicatively coupled with the first port of the scent dispensing device. Returning the first scent cartridge to the default orientation may include orienting the first scent cartridge such that one of the plurality of chambers in which an air filter is located is fluidly communicatively coupled with the first port of the scent dispensing device. The method may further include detecting a low available power condition of a power source via at least one controller, providing the indication of the stop condition in response to the detection of the low available power condition. The method may further include detecting a turn off input via at least one controller, and providing the indication of the stop condition in response to the detection of the turn off input. The method may further include stopping the operation of the air mover after orienting the first scent cartridge in the default orientation in response to the indication of the stop condition. The method may further include continuously operating the air mover to move air between the starting operation of the air mover and the stopping of the operation of the air mover in response to the indication of the stop condition.

A scent dispensing device may be summarized as including a first scent cartridge having a plurality of chambers at least some of the chambers having respective scent media therein; an air mover that moves air with respect to the first scent cartridge; a first motor that changes an orientation of the first scent cartridge; and at least one controller, wherein the at least one controller: starts operation of air mover with the first scent cartridge in a default orientation, following the starting operation of the air mover, causes the first motor to orient the first scent cartridge from the default orientation to a first orientation in which a respective outlet of a first one of the plurality of chambers of the first scent cartridge is fluidly communicatively coupled with a first port of the scent dispensing device, and in response to an indication of a stop condition, causes the first motor to return the first scent cartridge to the default orientation via the first motor.

The at least one controller may cause the first motor to return the first scent cartridge to the default orientation such that none of the plurality of chambers which contain scent media are fluidly communicatively coupled with the first port of the scent dispensing device. The at least one controller may cause the first motor to return the first scent cartridge to the default orientation such that one of the plurality of chambers which is empty is fluidly communicatively coupled with the first port of the scent dispensing device. The at least one controller may cause the first motor to return the first scent cartridge to the default orientation such that one of the plurality of chambers in which an air filter is located is fluidly communicatively coupled with the first port of the scent dispensing device. The at least one controller may detect a low available power condition of a power source, and may provide the indication of the stop condition in response to a detection of the low available power condition. The at least one controller may detect a turn off input, and may provide the indication of the stop condition in response to the detection of the turn off input. The at least one controller may cause the operation of the air mover to stop after causing the first motor to orient the first scent cartridge in the default orientation in response to the indication of the stop condition. The at least one controller may cause the air mover to continuously operate to move air between the starting operation of the air mover and the stopping of the operation of the air mover in response to the indication of the stop condition.

A method of operation in a scent sequence specification device may be summarized as including causing a presentation of a scent sequence specification user interface, the scent sequence specification user interface including a scent palette of a set of user selectable scent icons, each of the user selectable scent icons corresponding to a respective scent, the scent sequence specification user interface further including an array of scent specification fields which are selectively populatable via selections of the user selectable scent icons from the scent palette; and receiving inputs via the scent sequence specification user interface; and generating a scent specification file based at least in part on the inputs received via the scent sequence specification user interface.

Causing the presentation of the scent sequence specification user interface may include causing a presentation of the scent sequence specification user interface including two or more categories of scents. Causing the presentation of the scent sequence specification user interface may include causing a presentation of the scent sequence specification user interface including user selectable icons for two or more scents in each of the categories of scents. Causing the presentation of the scent sequence specification user interface may include causing a presentation of the scent sequence specification user interface including, in each of the categories of scents, two or more user selectable icons for two or more scents arranged spatially sequentially with respect to one another in the respective category. Causing the presentation of the scent sequence specification user interface may include causing a presentation of the scent sequence specification user interface including, in each of the categories of scents, two or more user selectable icons for two or more scents arranged spatially sequentially in columns with respect to one another in the respective category. Causing the presentation of the scent sequence specification user interface may include causing a presentation of a first array of user selectable scent icons each of which represents a respective scent of a first category of scent, and at least a second array of user selectable scent icons each of which represents a respective scent of a second category of scent, the second category of scent different from the first category of scent. Causing the presentation of the scent sequence specification user interface may include causing a presentation of at least two of the following: a first array of user selectable scent icons each of which represents a respective scent of a coffee category of scent, a second array of user selectable scent icons each of which represents a respective scent of a chocolate category of scent, a third array of user selectable scent icons each of which represents a respective scent of a caramel category of scent, or a fourth array of user selectable scent icons each of which represents a respective scent of a nut category of scent. Causing the presentation of the scent sequence specification user interface may include causing a presentation of the scent sequence specification user interface including an array of scent specification fields where a temporal sequence is represented by distribution of the scent specifications fields along a first axis of the array of scent specification fields. Causing the presentation of the scent sequence specification user interface may include causing a presentation of the scent sequence specification user interface including a two-dimensional array of scent specification fields having a first axis and second axis perpendicular to the first axis, where a distribution of the scent specifications fields along the first axis of the array represents a position in a temporal sequence and a distribution of the scent specification field along the second axis of the array represents one or more scents to be dispensed at a respective position in the temporal sequence. Causing the presentation of the scent sequence specification user interface may include causing a presentation of the scent sequence specification user interface including a name field, and may further include logically associating a name entered in the name field with the scent specification file. Causing the presentation of the scent sequence specification user interface may include causing a presentation of the scent sequence specification user interface including a recipient address field and a submit user selectable icon; and may further include in response to selection of the submit user selectable icon, causing the scent specification file to be transmitted to an address specified via the recipient address field. Causing a presentation of a scent sequence specification user interface may include providing a set of processor executable instructions to a processor-based device. Causing a presentation of a scent sequence specification user interface may include wirelessly providing at least one markup language file to a mobile communications device via a server computer. Causing a presentation of a scent sequence specification user interface may include providing a set of Web services via a server computer.

A scent sequence specification device may be summarized as including at least one processor; and a computer-readable storage medium storing instructions that, when executed by the at least one processor, cause the scent sequence specification device to: present a scent sequence specification user interface, the scent sequence specification user interface including a scent palette of a set of user selectable scent icons, each of the user selectable scent icons corresponding to a respective scent, the scent sequence specification user interface further including an array of scent specification fields which are selectively populatable via selections of the user selectable scent icons from the scent palette; and receiving inputs via the scent sequence specification user interface; and generating a scent specification file based at least in part on the inputs received via the scent sequence specification user interface.

The scent sequence specification user interface may include two or more categories of scents. The scent sequence specification user interface may include user selectable icons for two or more scents in each of the categories of scents. The scent sequence specification user interface may include, in each of the categories of scents, two or more user selectable icons for two or more scents arranged spatially sequentially with respect to one another in the respective category. The scent sequence specification user interface may include, in each of the categories of scents, two or more user selectable icons for two or more scents arranged spatially sequentially in columns with respect to one another in the respective category. The scent sequence specification user interface may include a first array of user selectable scent icons each of which represents a respective scent of a first category of scent, and at least a second array of user selectable scent icons each of which represents a respective scent of a second category of scent, the second category of scent different from the first category of scent. The scent sequence specification user interface may include at least two of the following: a first array of user selectable scent icons each of which represents a respective scent of a coffee category of scent, a second array of user selectable scent icons each of which represents a respective scent of a chocolate category of scent, a third array of user selectable scent icons each of which represents a respective scent of a caramel category of scent, or a fourth array of user selectable scent icons each of which represents a respective scent of a nut category of scent. The scent sequence specification user interface may include an array of scent specification fields where a temporal sequence is represented by distribution of the scent specifications fields along a first axis of the array of scent specification fields. The scent sequence specification user interface may include a two-dimensional array of scent specification fields having a first axis and second axis perpendicular to the first axis, where a distribution of the scent specifications fields along the first axis of the array represents a position in a temporal sequence and a distribution of the scent specification field along the second axis of the array represents one or more scents to be dispensed at a respective position in the temporal sequence. The scent sequence specification user interface may include a name field, the at least one processor logically associates a name entered in the name field with the scent specification file. The scent sequence specification user interface may include a recipient address field and submit user selectable icon and, in response to selection of the submit user selectable icon, the at least one processor may cause the scent specification file to be transmitted to an address specified via the recipient address field. The at least one processor may present the scent sequence specification user interface by providing a set of processor executable instructions to a processor-based device. The at least one processor may present the scent sequence specification user interface by wirelessly providing at least one markup language file to a mobile communications device. The at least one processor may present the scent sequence specification user interface by providing a set of Web services.

A consumable scent cartridge may be summarized as including a housing having at least one wall defining a peripheral perimeter, a first end, a second end opposed across a length of the housing from the first end, and a plurality of chambers in the housing annularly arrayed about a rotational axis thereof, at least two of the chambers extending through the first and the second ends, each of the chambers separated and distinct from one another; and a plurality of consumable scent media, a respective one of the consumable scent media received in a respective one of each of at least two of the chambers.

At least one of the chambers may be empty. 85. The consumable scent cartridge of claim 83 wherein at least one of the chambers may contain a medium that is unscented. At least one of the chambers may contain an air filter medium that removes scent from air that passes therethrough. Each of the chambers may have an aperture at least proximate the first end, the apertures each having a respective dimensional area, and the first end includes at least one portion spaced between two of the apertures, the at least one portion having a dimensional area equal to the respective dimensional area of the apertures. Each of the consumable scent media may be in a non-liquid form. Each consumable scent medium may be in a powder form. Each consumable scent medium may be in a packed powder form. Each consumable scent medium may be in a wax form. For each of the chambers, there may be a gap between the consumable scent medium and an inner surface of at least one wall of the respective chamber, the gap extending along the length of the housing. For each of the chambers, there may be a gap between the consumable scent medium and an inner surface of the at least one wall that defines the peripheral perimeter of the housing. The housing may have a circular peripheral perimeter and each chamber is a wedge-shaped segment of a circular profile of the circular peripheral perimeter. For each of the chambers, there may be a gap between the consumable scent medium and an inner surface of the at least one wall that defines the peripheral perimeter of the housing, and each consumable scent medium may be adjacent at least one other wall that forms the respective chamber. The consumable scent cartridge may further include at least one selective membrane at each of the first and the second ends, which at least one selective membrane retains consumable scent media in a solid form and passes air and scented air, and wherein, for each of the chambers, there is a gap between the consumable scent medium and all walls that form the respective chamber, the consumable scent media in contact with the respective selective membranes. The at least one selective membrane may have a nominal pore size small enough to retain consumable scent media in a solid form and large enough to allow passage of air and scented air. Each of the consumable scent media may have a respective scent, the scents of a respective scent medium received in each of the at least two of the chambers being different from one another. The housing may include an annular bearing surface at the second end of the housing, the annular bearing surface disposed about the axis of rotation. The consumable scent cartridge may further include a first and a second hermetically sealed membrane removably located respectively across the first and the second ends of the housing.

A consumable scent cartridge may be summarized as including a housing having at least one wall defining a peripheral perimeter, a first end, a second end opposed across a length of the housing from the first end, and a plurality of chambers, at least two of the chambers extending through the first and the second ends, each of the chambers separated and distinct from one another; and a plurality of consumable scent media, each of the consumable scent media received in respective ones of each of at least two of the chambers.

Each consumable scent medium may be in a solid form. Each consumable scent medium may be in a powder form. Each consumable scent medium may be in a packed powder form. Each consumable scent medium may be in a wax form. The consumable scent cartridge may further include at least one selective membrane at each of the first and the second ends, the selective membranes which retain the consumable scent media in the respective chambers while passing air into and scented air out of the respective chambers. For each of the chambers, there may be a gap between the consumable scent medium and all walls that form the respective chamber, the consumable scent media in contact with the respective selective membranes. The housing may have a circular peripheral perimeter and each chamber may be a wedge-shaped segment of a circular profile of the circular peripheral perimeter. Each of the consumable scent media may have a respective scent, the scents of a respective scent medium received in each of the at least two of the chambers being different from one another. The consumable scent cartridge may further include a first and a second hermetically sealed membrane removably located respectively across the first and the second ends of the housing to hermetically seal the chambers at least prior to use.

A consumable scent cartridge may be summarized as including a housing having a first end, a second end opposed across a length of the housing from the first end, and a plurality of chambers in the housing, each of the chambers separated and distinct from one another and having an aperture at least proximate the first end of the housing; a plurality of consumable scent media in a particulate form, a respective one of the consumable scent medium located in a respective one of each of at least two of the chambers; and at least one first end selective membrane at least proximate the first end of the housing and positioned across at least the apertures of the at least two of the chambers in which the consumable scent medium is located, the at least one first end selective membrane which selectively passes scented air while not passing the consumable scent media in the particulate form.

Each of the chambers additionally may have an aperture at least proximate the second end of the housing, and may further include at least one second end selective membrane at least proximate the second end of the housing and positioned across at least the apertures of the at least two of the chambers in which the consumable scent medium is located, the at least one second end selective membrane which selectively passes scented air while not passing the consumable scent media in the particulate form. Each of the chambers may form a fluid flow path within the housing, extending through the first and the second ends of the housing. Each consumable scent medium may be in a powder form. Each consumable scent medium may be in a packed powder form. Each consumable scent medium may be in a wax form. For each of the chambers, there may be a gap between the consumable scent medium and all walls that form the respective chamber, the consumable scent media in contact with at least one of the respective selective membranes. The consumable scent cartridge may further include a first and a second hermetically sealed membrane removably located respectively across the first and the second ends of the housing to hermetically seal the chambers at least prior to use. The first and the second hermetically sealed membranes may be foil membranes with a pressure sensitive adhesive about at least a peripheral portion thereof.

A consumable scent kit may be summarized as including a first consumable scent cartridge having a plurality of chambers, two or more of the chambers containing consumable scent media; and at least a second consumable scent cartridge having a plurality of chambers, two or more of the chambers containing consumable scent media in a non-liquid form, at least some of the consumable scent media of the second consumable scent cartridge having a scent that is a different scent from that of at least some of the consumable scent media of the first consumable scent cartridge.

At least one of the chambers may be empty. At least one of the chambers may contain a medium that is unscented. At least one of the chambers may contain a scent neutralizing medium. At least one of the chambers may contain an air filter medium that removes scent from air that passes therethrough. Each consumable scent medium may be in a non-liquid form. Each consumable scent medium may be a solid. Each consumable scent medium may be a reservoir of particulate. The reservoir of particulate may be a powder. The reservoir of particulate may be a packed powder. Each consumable scent medium may be in a wax form. The consumable scent kit may further include a first pair of hermetically sealed membranes removably located respectively across a first end and a second end of the first consumable scent cartridge to hermetically seal the chambers thereof at least prior to use; and a second pair of hermetically sealed membranes removably located respectively across a first end and a second end of the second consumable scent cartridge to hermetically seal the chambers thereof at least prior to use. The hermetically sealed membranes may be foil membranes each with a respective tab and with a pressure sensitive adhesive about at least a peripheral portion thereof, the pressure sensitive which allows resealing of the chambers after use. The consumable scent kit may further include a first hermetically sealed pouch which contains at least the first consumable scent cartridge prior to use. The first hermetically sealed pouch may also contain at least the second consumable scent cartridge prior to use. The consumable scent kit may further include a second hermetically sealed pouch which contains at least the second consumable scent cartridge prior to use, the first and the second hermetically sealed pouches packaged together prior to use. The consumable scent kit may further include a blister pack including a backing substrate and a blister substrate carried by the backing substrate, the blister substrate having a plurality of blisters to receive respective ones of the first and at the second first consumable scent cartridges therein prior to use. The consumable scent media contained in each of the chambers of the first consumable scent cartridge which contain consumable scent media may have a different scent from one another. The consumable scent media contained in each of the chambers of the first consumable scent cartridge which contain consumable scent media may have a same scent as each other.

A consumable scent cartridge may be summarized as including a housing having a first end, a second end opposed across a length of the housing from the first end, and a plurality of chambers in the housing, each of the chambers separated and distinct from one another and having an aperture at least proximate the first end of the housing; and a plurality of consumable scent media, a respective one of the consumable scent media located in a respective one of each of at least two of the chambers, with at least fifty percent of a total surface area of the consumable scent medium exposed to an airflow through the chamber.

Each consumable scent media may emit a scent at least during use and is in a non-liquid form. Each consumable scent media may be in a powder form. Each consumable scent media may be in a packed powder form. For each of the chambers, there may be a gap between the consumable scent medium and an inner surface of at least one wall of the respective chamber, the gap extending along the length of the housing. For each of the chambers, there may be a gap between the consumable scent medium and an inner surface of at least one wall that defines a peripheral perimeter of the housing. The housing may have a circular peripheral perimeter and each chamber may be a wedge-shaped segment of a circular profile of the circular peripheral perimeter. For each of the chambers, there may be a gap between each consumable scent media medium and an inner surface of at least one wall that defines a peripheral perimeter of the housing, and each consumable scent medium may be adjacent at least one other wall that forms the respective chamber. The consumable scent cartridge may further include at least one selective membrane at each of the first and the second ends, and wherein, for each of the chambers, there may be a gap between the consumable scent medium and all walls that form the respective chamber, the consumable scent media in contact with the respective selective membranes. The at least one selective membrane at each of the first and the second ends may include at least one first end selective membrane at least proximate the first end of the housing and positioned across at least the apertures of the at least two of the chambers in which the consumable scent medium is located, the at least one first end selective membrane which selectively passes air or scented air while not passing the consumable scent media in the particulate form. The consumable scent cartridge may further include a first and a second hermetically sealed membrane removably located respectively across the first and the second ends of the housing. The consumable scent cartridge may further include an asymmetric coupler to detachably couple the cartridge to an asymmetric portion of a drive shaft.

A scent dispensing device to dispense a scent from a removable scent cartridge, the removable scent cartridge including body having a plurality of chambers each having a respective outlet, the body of the removable scent cartridge including an exterior surface having a keyway, the scent dispensing device may be summarized as including: an air mover that moves air; a first motor including an output shaft; a coupling sleeve coupled to the output shaft of the first motor and rotatable thereby, the coupling sleeve defining a volume shaped and sized to receive at least a portion of the exterior surface of the body of the removable scent cartridge, the coupling sleeve including a protruding key that slidably engages with the keyway of the removable scent cartridge to releasably interconnect the coupling sleeve with the removable scent cartridge such that the output shaft of the first motor transmits rotary motion to the coupling sleeve which in turn transmits rotary motion to the removable scent cartridge, wherein the first motor orients the removable scent cartridge in a first orientation in which a respective outlet of a first one of the plurality of chambers of the removable scent cartridge is fluidly communicatively coupled with a first port of the scent dispensing device after operation of the air mover has been started; and a controller that determines a direction of movement and an amount of movement in the determined direction of movement from the first orientation to orient the removable scent cartridge in a second orientation in which a respective outlet of a second one of the plurality of chambers is fluidly communicatively coupled with the first port of the scent dispensing device, and that causes the first motor to orient the removable scent cartridge in the second orientation in which the respective outlet of the second one of the plurality of chambers is fluidly communicatively coupled with the first port of the scent dispensing device.

The coupling sleeve may include a base portion and a cylindrical wall extending upwardly therefrom, and the protruding key may be disposed on an interior surface of the cylindrical wall. The base portion of the coupling sleeve may include a plurality of apertures each aligned with a respective outlet of the plurality of chambers of the removable scent cartridge. The coupling sleeve may include a motor shaft coupling portion including an aperture shaped and sized to receive at least a portion of the output shaft of the first motor. The device may further include: a light source operatively coupled to the controller, wherein the controller causes the light source to emit light upon the occurrence of one or more conditions. The one or more conditions may include the scent dispensing device receiving a scent specification that specifies at least one scent. The controller may determine which of a first rotational direction and a second rotational direction provides a least amount of movement to move the removable scent cartridge from the first orientation to the second orientation, the second rotational direction opposite the first rotational direction. The controller may determine a number of steps by a stepper motor in the first rotational direction that will orient the removable scent cartridge in the second orientation from the first orientation. The controller may determine a number of steps by a stepper motor in the first rotational direction that will orient the removable scent cartridge in the second orientation from the first orientation. The controller may cause the first motor to move the removable scent cartridge from the first orientation in the determined direction of movement by the determined amount of movement. The first direction may be a first rotational direction, the first motor may be a stepper motor, and the controller may cause the first motor to move the removable scent cartridge in the first rotational direction by a determined first number of steps. The controller may cause the first motor to orient the removable scent cartridge in a default orientation in response to a first indication. The controller may cause the first motor to orient the removable scent cartridge in the default orientation such that none of the plurality of chambers which contain scent media are fluidly communicatively coupled with the first port of the scent dispensing device. The controller may cause the first motor to orient the removable scent cartridge in the default orientation such that a third one of the plurality of chambers which is empty is fluidly communicatively coupled with the first port of the scent dispensing device. The controller may cause the first motor to orient the removable scent cartridge in the default orientation such that a third one of the plurality of chambers in which an air filter is located is fluidly communicatively coupled with the first port of the scent dispensing device. The controller may detect a low available power condition of a power source and provide the first indication in response to a detection of the low available power condition. The controller may detect a turn off input and provide the first indication in response to a detection of the turn off input. The controller may stop the operation of the air mover after orienting the removable scent cartridge in the default orientation in response to the first indication. The controller may cause the air mover to continuously operate to move air between starting operation of the air mover and stopping operation of the air mover in response to the first indication. The controller: may determine a direction of movement and an amount of movement in the determined direction of movement from the second orientation to orient a third one of the plurality of chambers of the removable scent cartridge with the first port of the scent dispensing device via the controller, and may cause the first motor to orient the removable scent cartridge in a third orientation in which a respective outlet of the second one of the plurality of chambers is fluidly communicatively coupled with the first port of the scent dispensing device, and may cause the air mover to continuously operate to move air between starting operation of the air mover and stopping operation of the air mover in response to the first indication. The controller may cause the first motor to orient the removable scent cartridge in the default orientation between orienting the removable scent cartridge in the second orientation and orienting the removable scent cartridge in a third orientation. The controller may determine a sequence of orientations of the removable scent cartridge based at least in part on a scent specification, the sequence of orientations including at least the first and the second orientations. The controller: may determine a direction of movement and an amount of movement in the determined direction of movement from the default orientation to orient the removable scent cartridge in the first orientation; and may cause the first motor to orient the removable scent cartridge in the first orientation in which the respective outlet of the first one of the plurality of chambers is fluidly communicatively coupled with the first port of the scent dispensing device before orienting the removable scent cartridge in the second orientation. The device may further include: a communications module that wirelessly receives the scent specification via a radio, wherein the communications module receives the scent specification before the controller determines the sequence of orientations of the removable scent cartridge. The device may further include: a light source operatively coupled to the controller, wherein the controller causes the light source to emit light when the communications module receives the scent specification.

A scent dispensing device, a body having a top surface; a plurality of spaced apart scent dispensing subunits coupled to the body, each of the plurality of scent dispensing subunits may be summarized as including: an air mover that moves air; a motor that orients a scent cartridge in a first orientation in which a respective outlet of a first one of a plurality of chambers of the scent cartridge is fluidly communicatively coupled with a port of the scent dispensing subunit after operation of the air mover has been started; an elongate scent channel fluidly communicatively coupled with the port of the scent dispensing subunit, the elongate scent channel attached to and extending upwardly from the top surface of the body and being selectively rotatable about a vertical axis; and a controller that determines a direction of movement and an amount of movement in the determined direction of movement from the first orientation to orient the scent cartridge in a second orientation in which a respective outlet of a second one of the plurality of chambers is fluidly communicatively coupled with the port of the scent dispensing subunit, and that causes the motor to orient the scent cartridge in the second orientation in which the respective outlet of the second one of the plurality of chambers is fluidly communicatively coupled with the port of the scent dispensing subunit.

The body of the scent dispensing device may include a lower body portion, and the top surface may be rotatably coupled to the lower body portion. The plurality of spaced apart scent dispensing units may include two spaced apart scent dispensing units, and each of the elongate scent channels of the respective scent dispensing units may be rotatable over a range of at least 90 degrees.

A scent dispensing device to dispense a scent from a removable scent cartridge, the removable scent cartridge including body having a plurality of chambers each having a respective outlet, the body of the removable scent cartridge including an exterior surface having a keyway, the scent dispensing device may be summarized as including: a scent channel comprising an air mover that moves air, the scent channel further comprising a first port; a first motor comprising an output shaft; a coupling sleeve coupled to the output shaft of the first motor and rotatable thereby, the coupling sleeve defining a volume shaped and sized to receive at least a portion of the exterior surface of the body of the removable scent cartridge, the coupling sleeve including a protruding key that slidably engages with the keyway of the removable scent cartridge to releasably interconnect the coupling sleeve with the removable scent cartridge such that the output shaft of the first motor transmits rotary motion to the coupling sleeve which in turn transmits rotary motion to the removable scent cartridge, wherein the first motor orients the removable scent cartridge in a first orientation in which a respective outlet of a first one of the plurality of chambers of the removable scent cartridge is fluidly communicatively coupled with the first port of the scent channel after operation of the air mover has been started; and a controller that determines a direction of movement and an amount of movement in the determined direction of movement from the first orientation to orient the removable scent cartridge in a second orientation in which a respective outlet of a second one of the plurality of chambers is fluidly communicatively coupled with the first port of the scent channel, and that causes the first motor to orient the removable scent cartridge in the second orientation in which the respective outlet of the second one of the plurality of chambers is fluidly communicatively coupled with the first port of the scent channel.

The device may further include: a base portion that houses the first motor, the coupling sleeve and the controller; and a lid portion, wherein the scent channel is coupled to the lid portion, and the lid portion is rotatably coupled to the base portion. The scent channel may include a light source operatively coupled to the controller, and the controller may cause the light source to emit light upon the occurrence of one or more conditions.

A scent dispensing device to dispense a scent from a removable scent cartridge, the removable scent cartridge including body having a plurality of chambers each having a respective outlet, the body of the removable scent cartridge including an exterior surface having a first coupling portion, the scent dispensing device may be summarized as including: an air mover that moves air; a first motor comprising an output shaft; a coupling sleeve coupled to the output shaft of the first motor and rotatable thereby, the coupling sleeve defining a volume shaped and sized to receive at least a portion of the exterior surface of the body of the removable scent cartridge, the coupling sleeve including a second coupling portion that engages the first coupling portion of the removable scent cartridge to releasably interconnect the coupling sleeve with the removable scent cartridge such that the output shaft of the first motor transmits rotary motion to the coupling sleeve which in turn transmits rotary motion to the removable scent cartridge, wherein the first motor orients the removable scent cartridge in a first orientation in which a respective outlet of a first one of the plurality of chambers of the removable scent cartridge is fluidly communicatively coupled with a first port of the scent dispensing device after operation of the air mover has been started; and a controller that determines a direction of movement and an amount of movement in the determined direction of movement from the first orientation to orient the removable scent cartridge in a second orientation in which a respective outlet of a second one of the plurality of chambers is fluidly communicatively coupled with the first port of the scent dispensing device, and that causes the first motor to orient the removable scent cartridge in the second orientation in which the respective outlet of the second one of the plurality of chambers is fluidly communicatively coupled with the first port of the scent dispensing device.

A consumable scent cartridge may be summarized as including: a housing having at least one wall defining a peripheral perimeter, a first end, a second end opposed across a length of the housing from the first end, and a plurality of chambers in the housing arrayed about a rotational axis thereof, at least two of the chambers extending through the first and the second ends, each of the chambers separated and distinct from one another, the wall comprising one of a key and a keyway that mates with the other of a key and a keyway of a coupling portion of a scent dispensing device to define a keyed joint, the keyed joint permitting axial movement between the housing of the consumable scent cartridge and the coupling portion of the scent dispensing device and restricting rotational movement therebetween about the rotational axis of housing; and a plurality of consumable scent media, a respective one of the consumable scent media received in a respective one of each of at least two of the chambers.

The wall may include a keyway extending upwardly along the wall from the second end.

A consumable scent cartridge may be summarized as including: a housing having at least one wall defining a peripheral perimeter, a first end, a second end opposed across a length of the housing from the first end, and a plurality of chambers in the housing arrayed about a rotational axis thereof, at least two of the chambers extending through the first and the second ends, each of the chambers separated and distinct from one another, the wall comprising a recess that mates with a protrusion of a coupling portion of a scent dispensing device to define a keyed joint, the keyed joint permitting axial movement between the housing of the consumable scent cartridge and the coupling portion of the scent dispensing device and restricting rotational movement therebetween about the rotational axis of housing; and a plurality of consumable scent media, a respective one of the consumable scent media received in a respective one of each of at least two of the chambers.

A consumable scent cartridge may be summarized as including: a housing having at least one wall defining a peripheral perimeter, a first end, a second end opposed across a length of the housing from the first end, and a plurality of chambers in the housing arrayed about a rotational axis thereof, at least two of the chambers extending through the first and the second ends, each of the chambers separated and distinct from one another, the wall comprising exterior coupling portion that mates with interior coupling portion of a coupling sleeve of a scent dispensing device to define a keyed joint, the keyed joint permitting axial movement between the housing of the consumable scent cartridge and the coupling portion of the scent dispensing device and restricting rotational movement therebetween about the rotational axis of housing; and a plurality of consumable scent media, a respective one of the consumable scent media received in a respective one of each of at least two of the chambers.

### BRIEF DESCRIPTION OF THE SEVERAL VIEWS OF THE DRAWINGS

FIG. 1A is an isometric view of a scent dispensing device, according to one illustrated embodiment.

FIG. 1B is an exploded view of the scent dispensing device of FIG. 1A.

FIG. 2A is an isometric view of a scent emitter, according to one illustrated embodiment.

FIG. 2B is an exploded view of the scent emitter of FIG. 2A.

FIG. 3A is a front elevational view of a first diffuser part, according to one illustrated embodiment.

FIG. 3B is a rear plan view of the first diffuser part of FIG. 3A.

FIG. 3C is a top plan view of the first diffuser part of FIG. 3A, with a fan attached thereto.

FIG. 4 is a front elevational view of a second diffuser part, according to one illustrated embodiment.

FIG. 5A is a front elevational view of a scent mixing chamber according to one illustrated embodiment.

FIG. 5B is a bottom plan view of the scent mixing chamber of FIG. 5A.

FIG. 6A is a top plan view of an upper shelf of the scent dispensing device of FIG. 1A.

FIG. 6B is an enlarged view of a portion of the upper shelf of FIG. 6A.

FIG. 7A is a side elevational view of a motor, according to one illustrated embodiment.

FIG. 7B is a top plan view of the motor of FIG. 7A.

FIG. 8A is a top plan view of a lower shelf of the scent dispensing device of FIG. 1A.

FIG. 8B is an enlarged view of a portion of the lower shelf of FIG. 8A.

FIG. 9A is a front elevational view of a scent cartridge, according to one illustrated embodiment.

FIG. 9B is cross-sectional view taken along line I-I of FIG. 9A.

FIG. 9C is a top plan view the scent cartridge of FIG. 9A.

FIG. 9D is top plan view of the scent cartridge of FIG. 9A, with an end portion thereof removed.

FIG. 10A is an isometric view of a scent dispensing device according to one illustrated embodiment.

FIG. 10B is a partially exploded view of the scent dispensing device of FIG. 10A.

FIG. 10C is another partially exploded view of the scent dispensing device of FIG. 10A.

FIG. 10D is cross-sectional view taken along line II-II of FIG. 10A.

FIG. 10E is bottom plan view of a scent mixing chamber of the scent dispensing device of FIG. 10A.

FIG. 11A is a top plan view of a scent cartridge holder of the scent dispensing device of FIG. 10A.

FIG. 11B is an isometric view of the scent cartridge holder of FIG. 11A.

FIG. 12A is an isometric view of a scent cartridge of the scent dispensing device of FIG. 10A.

FIG. 12B is a partially exploded view of the scent cartridge of FIG. 12A.

FIG. 12C is a bottom plan view of the scent cartridge of FIG. 12A.

FIG. 13A is a horizontal slice view taken along line III-III of FIG. 12A, according to one illustrated embodiment.

FIG. 13B is a horizontal slice view taken along line III-III of FIG. 12A, according to one illustrated embodiment.

FIG. 13C is a horizontal slice view taken along line III-III of FIG. 12A, according to one illustrated embodiment.

FIG. 13D is a horizontal slice view taken along line III-III of FIG. 12A, according to one illustrated embodiment.

FIG. 13E is a horizontal slice view taken along line III-III of FIG. 12A, according to one illustrated embodiment.

FIG. 13F is a horizontal slice view taken along line III-III of FIG. 12A, according to one illustrated embodiment.

FIG. 14A is an isometric view of an end portion of a scent cartridge, according to one illustrated embodiment.

FIG. 14B is an exploded view of the end portion of the scent cartridge of FIG. 14A.

FIG. 14C is a bottom plan view of a hermetically sealed membrane of the end portion of the scent cartridge of FIG. 14A, according to one illustrated embodiment.

FIG. 15A an exploded view of a scent cartridge pouch, according to one illustrated embodiment.

FIG. 15B is an exploded view of a scent cartridge blister pack, according to one illustrated embodiment.

FIG. 16 is a network diagram of a scent dispensing system according to one illustrated embodiment.

FIGS. 17A-17F illustrate a schematic diagram of a scent dispensing device, according to one illustrated embodiment.

FIG. 18 is a block diagram of a mobile device, according to one illustrated embodiment.

FIG. 19 is a block diagram of a server, according to one illustrated embodiment.

FIG. 20 is block diagram of a scent sequence specification user interface, according to one illustrated embodiment.

FIG. 21 is a flowchart of a method of operating a scent dispensing system, according to one illustrated embodiment.

FIG. 22 is a flowchart of a method of operating a scent dispensing system, according to one illustrated embodiment.

FIG. 23 is a flowchart of a method of operating a scent dispensing system, according to one illustrated embodiment.

FIG. 24A is a top plan view of a scent cartridge, according to one illustrated embodiment.

FIG. 24B is a top plan view of the scent cartridge of FIG. 24A rotated clockwise by 144°.

FIG. 24C is a top plan view of the scent cartridge of FIG. 24B rotated clockwise by 72°.

FIG. 25 is a front perspective view of a scent dispensing device, according to one illustrated embodiment.

FIG. 26 is a rear perspective view of the scent dispensing device of FIG. 25.

FIG. 27 is a rear perspective, partially exploded view of portions of the scent dispensing device of FIG. 25.

FIG. 28 is a front perspective, partially exploded view of portions of the scent dispensing device of FIG. 25.

FIG. 29 is a front perspective view of the scent dispensing device of FIG. 25 when a lid thereof is in an open position.

FIG. 30 is an enlarged perspective view of a portion of the scent dispensing device of FIG. 25 when the lid is in the open position.

FIG. 31A is a front elevational view of the scent dispensing device of FIG. 25 when the lid is in the open position.

FIG. 31B is a sectional elevational view of the scent dispensing device taken along the line C-C of FIG. 31A.

FIG. 32A is a front elevational view of the scent dispensing device of FIG. 25 when the lid is in a closed position.

FIG. 32B is a sectional elevational view of the scent dispensing device taken along the line A-A of FIG. 32A.

FIG. 33A is a top plan view of a scent emitter of the scent dispensing device of FIG. 25.

FIG. 33B is an elevational sectional view of the scent emitter taken along the line A-A of FIG. 33A.

FIG. 34A is a perspective view, from the top, of a scent cartridge, according to one illustrated embodiment.

FIG. 34B is a perspective view, from the bottom, of the scent cartridge of FIG. 34A.

FIG. 34C is a front elevational view of the scent cartridge of FIG. 34A.

FIG. 34D is a bottom plan view of the scent cartridge of FIG. 34A.

FIG. 35A is an elevational view of the scent cartridge of FIG. 34A disposed above a scent cartridge receiving portion of the scent dispensing device of FIG. 25.

FIG. 35B is an elevational view of the scent cartridge of FIG. 34A disposed within the scent cartridge receiving portion of the scent dispensing device of FIG. 25.

FIG. 35C is a sectional bottom view taken along the line 35C-35C of FIG. 35B.

FIG. 35D is a bottom plan view of the scent cartridge receiving portion of the scent dispensing device of FIG. 25.

FIG. 35E is an isometric view of four scent cartridges disposed above four scent cartridge receiving portions of the scent dispensing device of FIG. 25.

FIG. 36A is a front perspective view of a scent dispensing device, according to one illustrated embodiment.

FIG. 36B is a front perspective view of the scent dispensing device of FIG. 36A when two scent emitters thereof are rotated inward by 45°.

FIG. 36C is a front perspective view of the scent dispensing device of FIG. 36A when two scent emitters thereof are rotated outward by 45°.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with microcontrollers, motor devices, power supplies such as DC/DC converters, wireless radios (i.e., transmitters, receivers or transceivers), computing systems including client and server computing systems, and networks (e.g., cellular, packet switched), as well as other communications channels, have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

The structure of various embodiments of a scent dispensing device will be described first. A scent dispensing system will then be described. The structure of various components of scent dispensing system will be described next. Several examples of operating of the scent dispensing system will then be described.

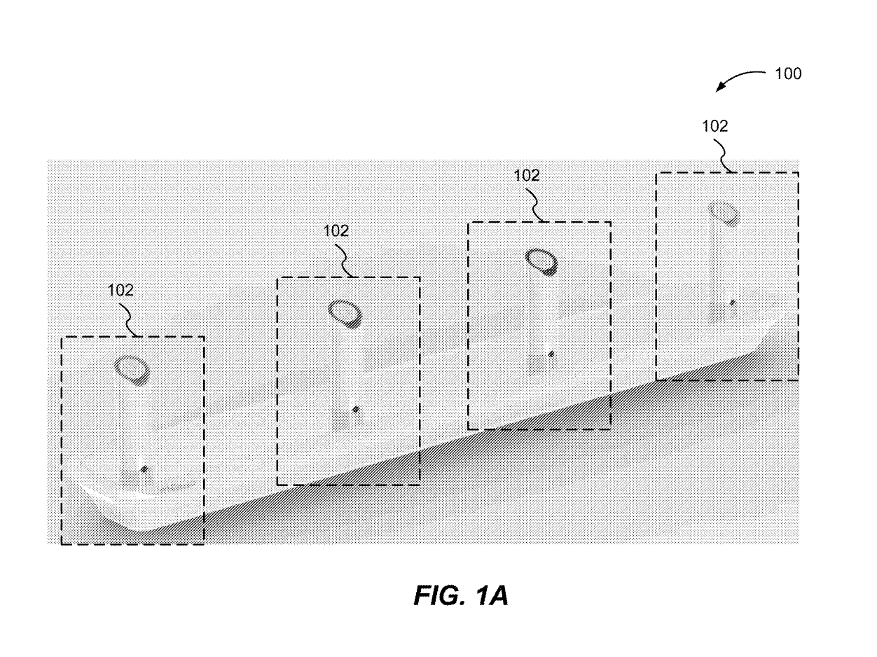

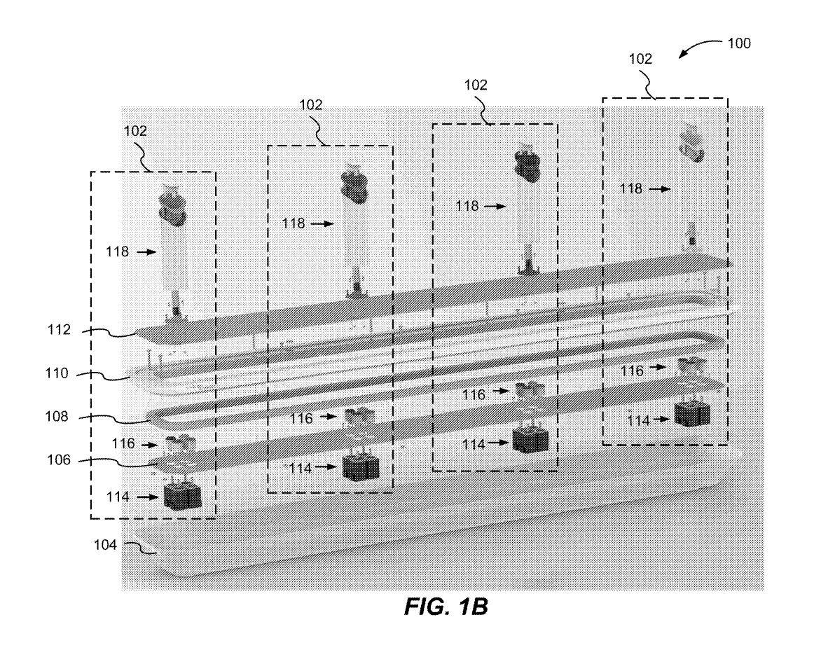

FIG. 1A is an isometric view of a scent dispensing device 100 according to one illustrated embodiment. FIG. 1B is an exploded view of the scent dispensing device 100. The scent dispensing device 100 includes four scent dispensing units 102, a base 104, a lower shelf 106, a spacer 108, a retaining member 110, and an upper shelf 112. Each scent dispensing unit 102 includes four motors 114, four scent cartridges 116, a scent emitter 118, and a number of electronic components that will be described below.

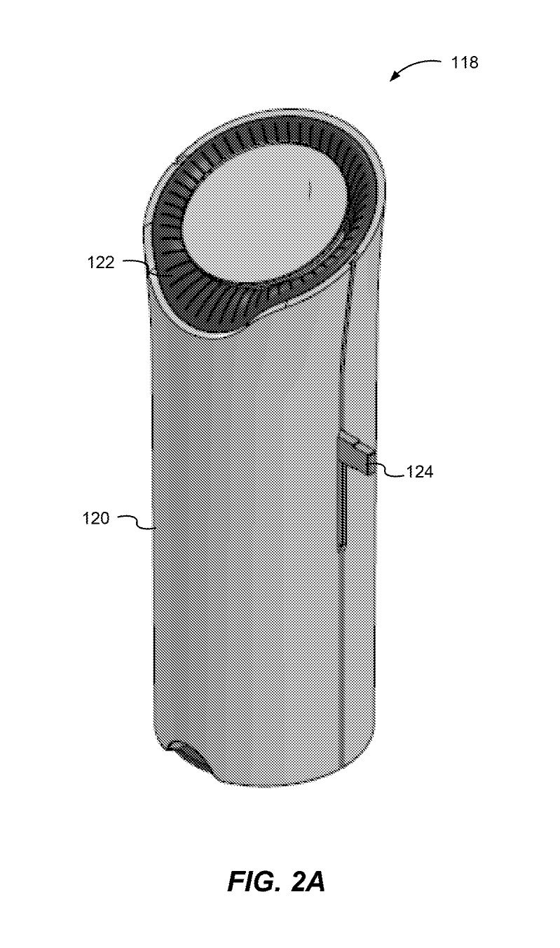

FIG. 2A is an isometric view of the scent emitter 118 (also referred to as a scent channel) according to one illustrated embodiment. FIG. 2B is an exploded view of the scent emitter 118 shown in FIG. 2A. The scent emitter 118 includes a housing 120 having a first housing portion 120a and a second housing portion 120b, a diffuser 122, a switch 124 that is used to control operation of a fan 126, a communications interface connector 128, a scent mixing chamber 130, and a conduit 132. In one embodiment, the communications interface connector 128 is a USB A-Type, female connector. The diffuser 122 includes a first diffuser portion 134, a second diffuser portion 136, and a third diffuser portion 138.

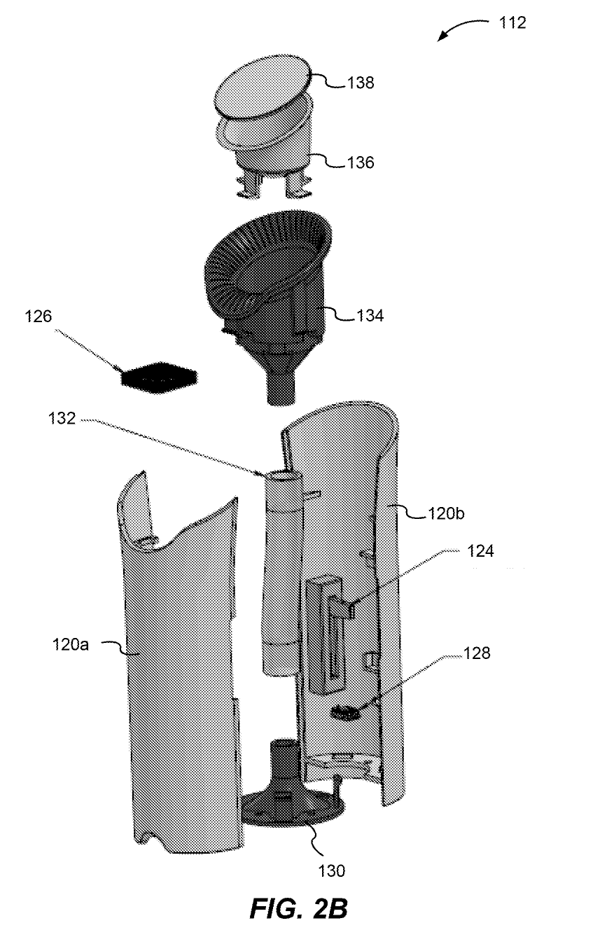

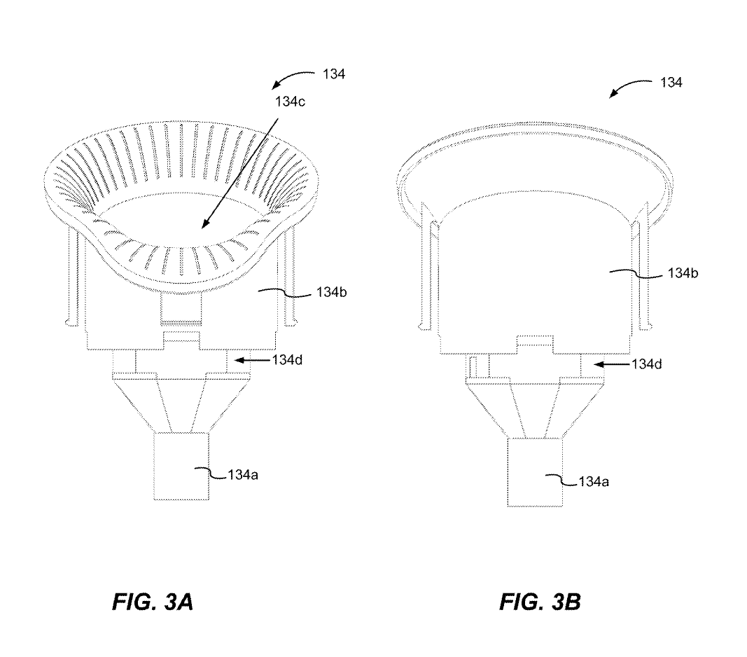

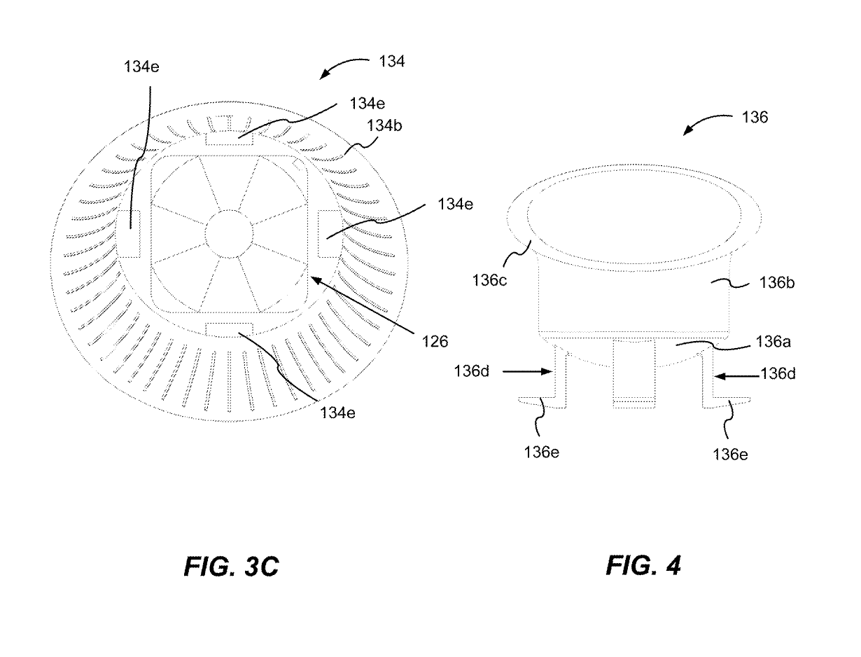

FIGS. 3A-3C show various views of the first diffuser portion 134 shown in FIG. 2B. The first diffuser portion 134 includes an inlet port 134a and a chamber 134b that defines an outlet port 134c. The inlet port 134a of the first diffuser portion 134 serves as an inlet port of the diffuser 122 and the outlet port 134c serves as an outlet port of the diffuser 122. A compartment 134d is disposed between the inlet port 134a and the outlet port 134c. The compartment 134d is sized and dimensioned to hold at least part of the fan 126 therein. The fan 126 is operable to cause air to flow from the inlet port 134a to the outlet port 134c of the first diffuser portion 134. As shown in FIG. 3C, the first diffuser portion 134 includes four rectangular apertures 134e formed in a lower portion of the chamber 134b.

FIG. 4 is a front elevational view of the second diffuser portion 136 shown in FIG. 2B. The second diffuser portion 136 includes a lower portion 136a, an upper portion 136b, and a lip 136c that extends outwardly from the periphery of the top of the upper portion 136b. Four retaining members 136d extend outwardly from the lower portion 136a of the second diffuser portion 136d; only three of the retaining members 136d can be seen in FIG. 4. A tapered portion 136e extends outwardly from each of the retaining members 136d.

The diffuser 122 is assembled by attaching the first diffuser portion 134 to the second diffuser portion 136, and by attaching the third diffuser portion 138 to the second diffuser portion 136. More particularly, the second diffuser portion 136 is placed inside the first diffuser portion 134, the retaining members 136d are aligned with the rectangular apertures 134e formed in a lower portion of the chamber 134b, and the second diffuser portion 136 is advanced toward the first diffuser portion 134 until the tapered portions 136e extend through the rectangular apertures 134e. The third diffuser portion 138 may be attached to the lip 136c of the second diffuser portion 136 using an adhesive. For example, an epoxy resin is placed on the top surface of the lip 136c, the third diffuser portion 138 is placed above the second diffuser portion 136, and the third diffuser portion 138 is advanced toward the second diffuser portion 136 until the bottom surface of the third diffuser portion 138 contacts the epoxy resin on the top surface of the lip 136c.

The first diffuser portion 134 and the second diffuser portion 136 are sized and dimensioned such that, when the second diffuser portion 136 is attached to the first diffuser portion 134, a passage is formed between the interior surfaces of the chamber 134b of the first diffuser portion 134 and the exterior surfaces of the second diffuser portion 136. When the scent emitter 118 is assembled and operated, the fan 126 causes air to flow into the inlet port 134a of the first diffuser portion 134, around the exterior surfaces of the second diffuser portion 136, and out of the outlet port 134c of the first diffuser portion 134.

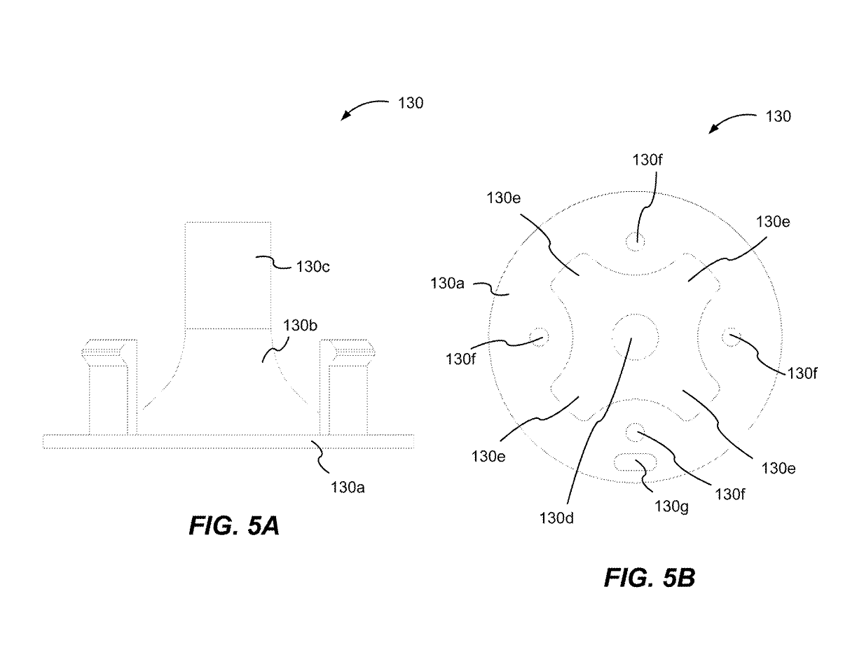

FIGS. 5A and 5B are front and bottom plan views, respectively, of the scent mixing chamber 130. The scent mixing chamber 130 includes a bottom portion 130a, a center portion 130b, and a top portion 130c that serves as an outlet port for the scent mixing chamber 130. As shown in FIG. 5B, the scent mixing chamber 130 includes a plurality of channels 130e that extend from the bottom portion 130a through the center portion 130b to the top portion 130c. The bottom portion 130a of the scent mixing chamber 130 includes four circular apertures 130f and an oval-shaped aperture 130g.

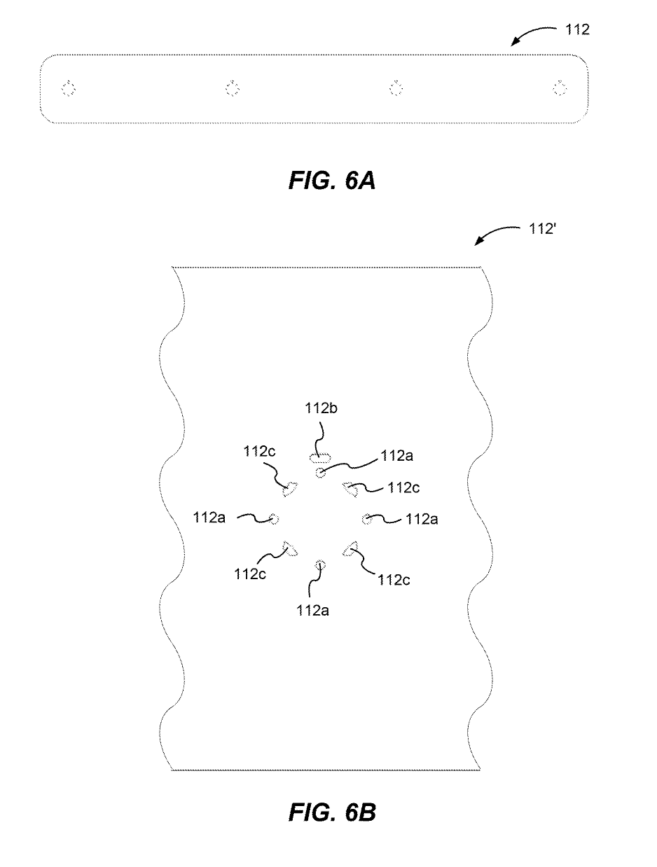

FIG. 6A is a top plan view of the upper shelf 112 of the scent dispensing device 100. FIG. 6B is an enlarged view of a portion 112′ of the upper shelf 112 of the scent dispensing device 100. The portion 112′ of the upper shelf 112 includes four circular apertures 112a, an oval-shaped aperture 112b, and four wedge-shaped apertures 112c.

Four scent mixing chambers 130 are attached to the upper shelf 112 using a plurality of nuts and bolts (not labeled). A process for attaching one of the scent mixing chambers 130 to the upper shelf 112 will now be described with reference to FIGS. 1B, 5A, 5B, 6A, and 6B. The scent mixing chamber 130 is placed on a top surface of the upper shelf 112 such that the circular apertures 130f of the scent mixing chamber 130 are aligned with a group of four circular apertures 112a of the upper shelf 112. Four bolts are inserted into respective circular apertures 130f of the scent mixing chamber 130 and passed through corresponding circular apertures 112a of the upper shelf 112. A nut is then secured to each bolt. The above-described process is repeated for each of the remaining scent mixing chambers 130. When the scent mixing chambers 130 are attached to the upper shelf 112, the wedge-shaped apertures 112c of the upper shelf 112 act as inlet ports to the scent mixing chambers 130.

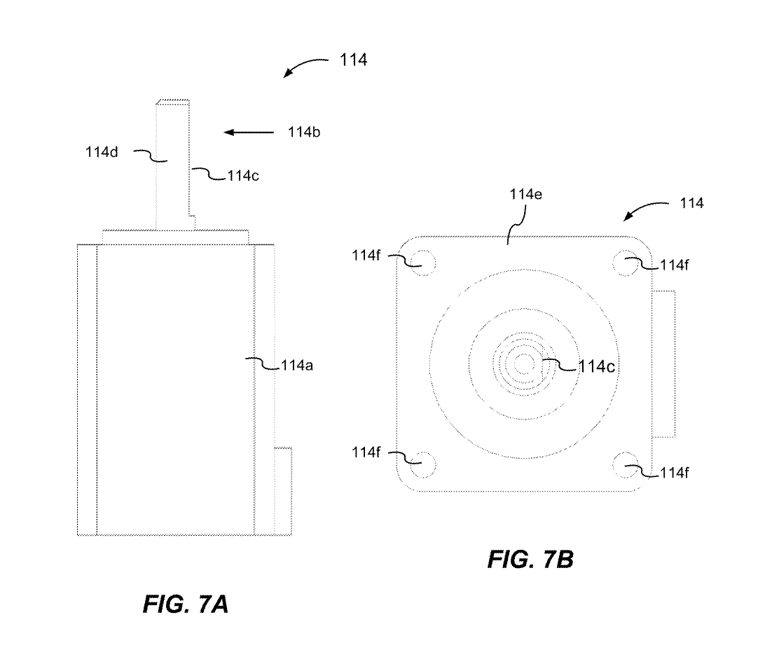

FIG. 7A is a side elevational view of the motor 114 according to one illustrated embodiment. The motor 114 includes a motor housing 114a and a shaft 114b that extends from the motor housing 114a. The shaft 114b includes a flat portion 114c and a truncated cylindrical portion 114d. As shown in FIG. 7B, the motor 114 includes a top portion 114e having a plurality of apertures 114f.

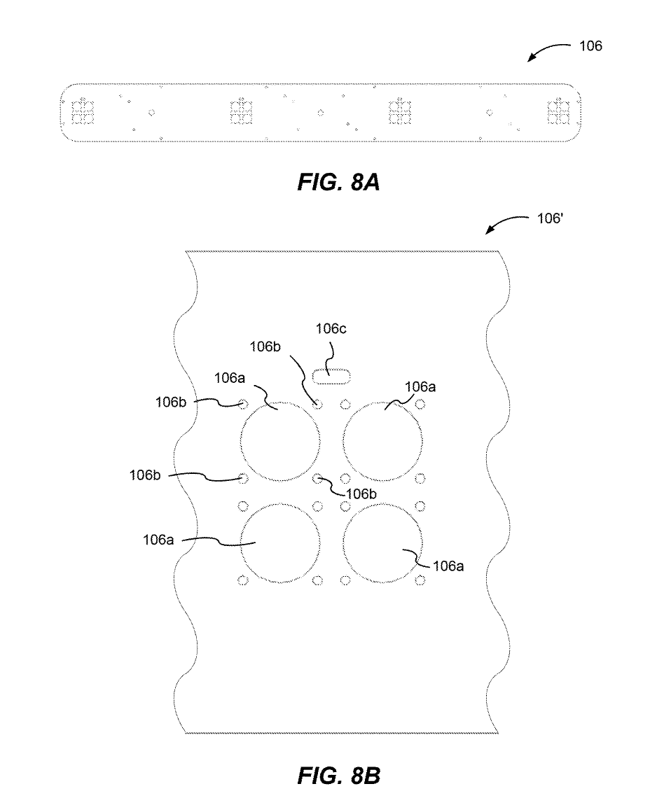

FIG. 8A is a top plan view of the lower shelf 106 of the scent dispensing device 100. FIG. 8B is an enlarged view of a portion 106′ of the upper shelf 106 shown in FIG. 8A. The portion 106′ of the lower shelf 106 includes four large circular apertures 106a, sixteen small circular apertures 106b, and an oval-shaped aperture 106c.

Sixteen motors 114 are attached to the lower shelf 106 using a plurality of bolts (not labeled). A process for attaching one of the motors 114 to the lower shelf 106 will now be described with reference to FIGS. 1B, 7A, 7B, 8A, and 8B. One of the motors 114 is placed below the lower shelf 106 such that the circular apertures 114f of the motor 114 are aligned with a group of four small circular apertures 106b surrounding one of the large circular apertures 106a of the lower shelf 106. The motor 114 is advanced toward the lower shelf 106 and the shaft 114b is passed through the large circular aperture 106a until the top portion 114e of the motor 114 abuts the bottom surface of the lower shelf 106. Four bolts are inserted through respective small circular apertures 106b of the lower shelf 106 and are advanced into the circular apertures 114f of the motor 114. The bolts are rotated to engage threads (not shown) within the motor housing 114a thereby attaching the motor 114 to the lower shelf 106. The above-described process is repeated for the remaining fifteen motors 114.

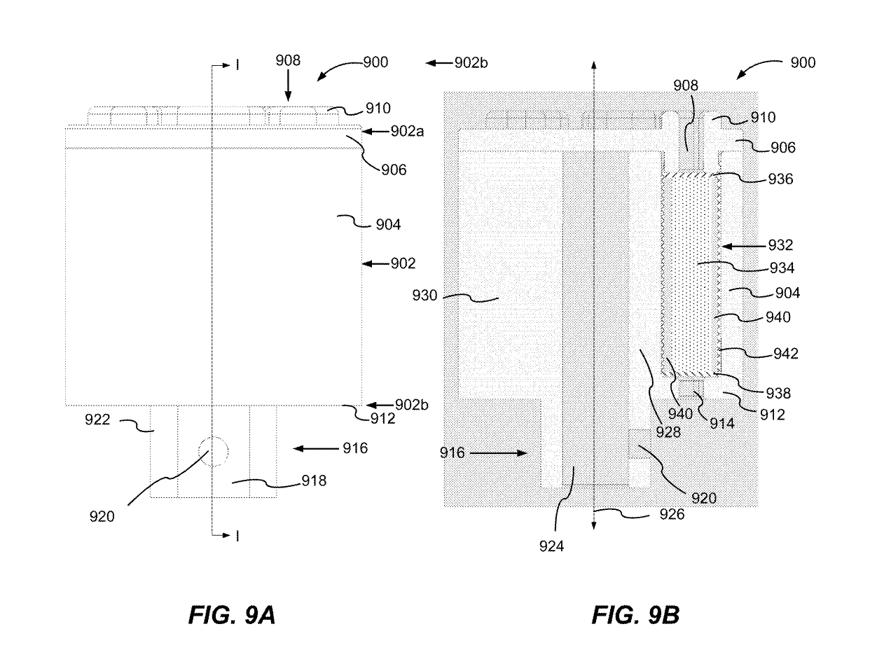

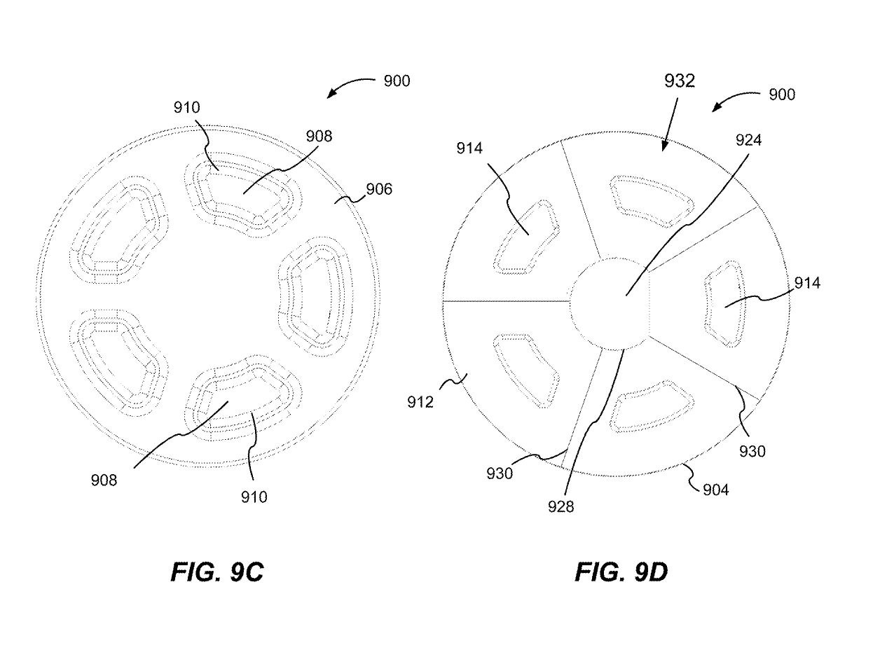

A scent cartridge 900 according to one illustrated embodiment will now be described with reference to FIGS. 9A-9D. FIG. 9A is a front elevational view of the scent cartridge 900. FIG. 9B is cross-sectional view taken along line I-I of FIG. 9A. FIG. 9C is a top plan view the scent cartridge 900. FIG. 9D is top plan view of the scent cartridge of FIG. 900, with a first end portion 906 removed.

As shown in FIG. 9A, the scent cartridge 900 includes a housing 902 having a first end 902a and a second end 902b. A cylindrical outer wall 904 defines a peripheral perimeter of the housing 902. A first end portion 906 is disposed at the first end 902a of the scent cartridge 900. The first end portion 906 includes five wedge-shaped apertures 908. A raised lip 910 extends outwardly from the first end portion 906 around the periphery of each of the apertures 908.

A second end portion 912 is disposed at the second end 902b of the scent cartridge 900. As best shown in FIG. 9D, the second end portion 912 includes five wedge-shaped apertures 914. As best shown in FIG. 9A, an axle 916 extends outwardly from the second end portion 912. The axle 916 includes a planar portion 918 having a circular aperture 920. The axle 916 also includes a truncated cylindrical portion 922. The planar portion 918 and the truncated cylindrical portion 922 form a “D” shaped aperture 924. The aperture 924 of the axle 916 is sized and dimensioned such that the shaft 114b of the motors 114 can be inserted into the aperture 924. When the shaft 114b of one of the motors 114 is inserted into the aperture 924 of the axle 916, the planar portion 114c of the shaft 114b is adjacent the planar portion 918 of the axle 916, which ensures that rotation of the shaft 114d causes rotation of the scent cartridge 900 about a longitudinal axis 926 of the scent cartridge 900.

FIG. 9C is a top view of the scent cartridge 900, and FIG. 9D is a top view of the scent cartridge 900 with the first end portion 906 removed. A plurality of walls extends upwardly from the second end portion 912, including the outer wall 904, an axial wall 928, and five planar walls 930. The planar walls 930 extend radially from the axial wall 928 to the outer wall 904. The first end portion 906, the second end portion 912, the planar walls 930, the axial wall 928, and the outer wall 904 define five chambers 932 within the scent cartridge 900. For each of the chambers 932, one of the apertures 908 formed in the first end portion 906 is aligned with a corresponding aperture 914 formed in the second end portion 912.

Referring now to FIG. 9B, a cross-sectional view of the scent cartridge 900 taken along line I-I of FIG. 9A is shown. Only one of the five chambers 932 can be seen in FIG. 9B. The scent cartridge 900 may have virtually any desired number of scent cartridge chambers 932. One or more of the chambers 932 may be filled with a scent medium 934. The scent medium 934 may be a non-liquid substance such as aromatic particles, a scented powder, which may be in a packed powder form, a scented wax, an air filter, or an unscented material. One or more of the chambers 932 may be empty.

A first selective membrane 936 may be attached to the bottom surface of the first end portion 906 of the scent cartridge 900. The first selective membrane 936 includes apertures (not shown) formed therein that are sized and dimensioned to enable the molecules of the scent medium 934 to flow from the apertures 908 of the first end portion 906 of the scent cartridge 900. For example, the scent medium 934 may be in a particulate form and the first selective membrane 936 selectively passes air or scented air while not passing the scent medium 934 in the particulate form. In one embodiment, the scent cartridge 900 does not include the first selective membrane 936.

A second selective membrane 938 may be attached to the upper surface of the second end portion 912. The second selective membrane 938 may include apertures (not shown) formed therein that are sized and dimensioned to prevent the scent medium 934 from falling out of the apertures 914, and to enable air to flow through the apertures 914. In one embodiment, the second selective membrane 938 is a non-porous plastic material. In another embodiment, the scent cartridge 900 does not include the second selective membrane 938. In one embodiment, the first selective membrane 936 and the second selective membrane 938 are formed from a cotton material having a thread count of 56 threads per centimeter.

As shown in FIG. 9B, the scent medium 934 may be positioned in the chamber 932 such that one or more gaps 940 are formed along the length of the housing 902 between one or more of the walls that define the chamber 932 and the scent medium 934. In FIG. 9B, one of the gaps 940 is formed between the scent medium 934 and the outer wall 904, and one of the gaps is formed between the scent medium 934 and the axial wall 928. The gaps 940 facilitate air flow through the chamber 932.

In one embodiment, one or more interior surfaces of one or more of the walls that define one or more of the scent cartridge chambers 932 have a coating 942 (e.g., wax) formed thereon. In FIG. 9B, the coating 942 is formed on the interior surfaces of the outer wall 904 and the axial wall 928.

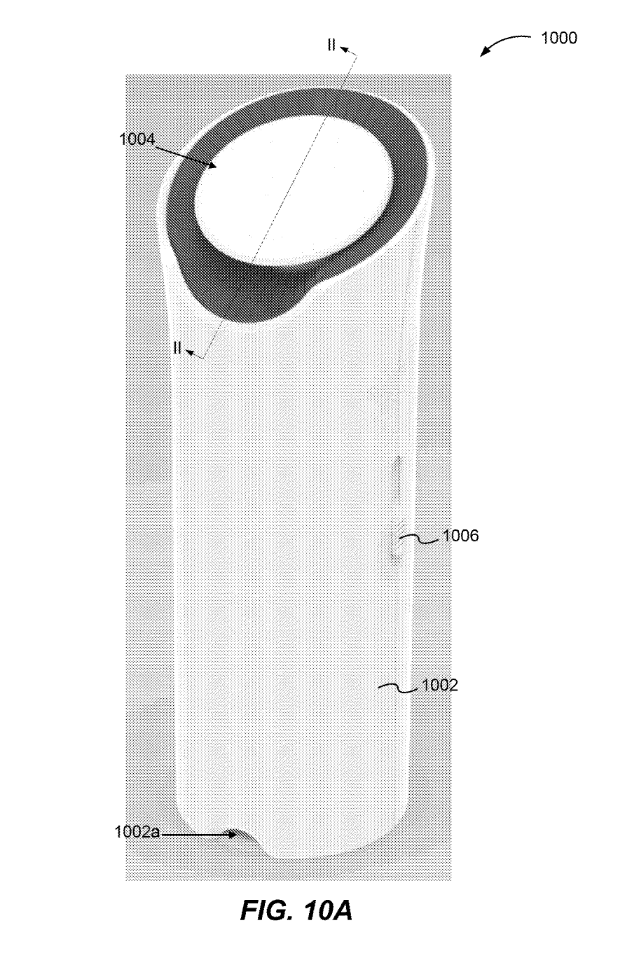

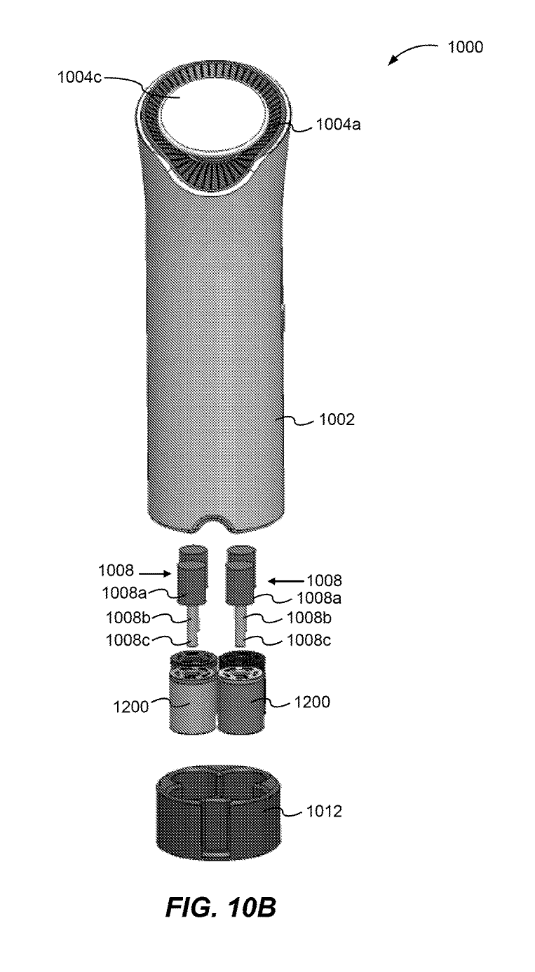

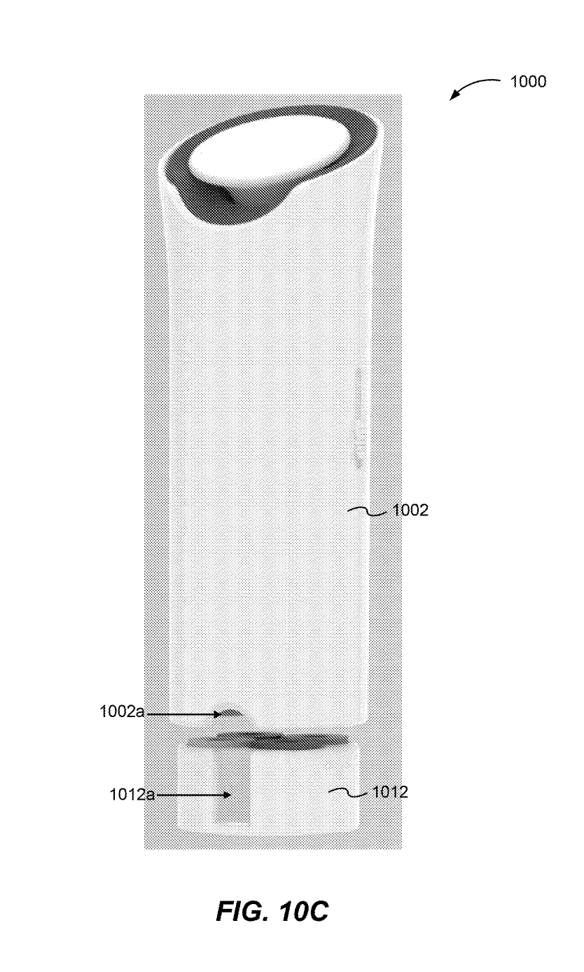

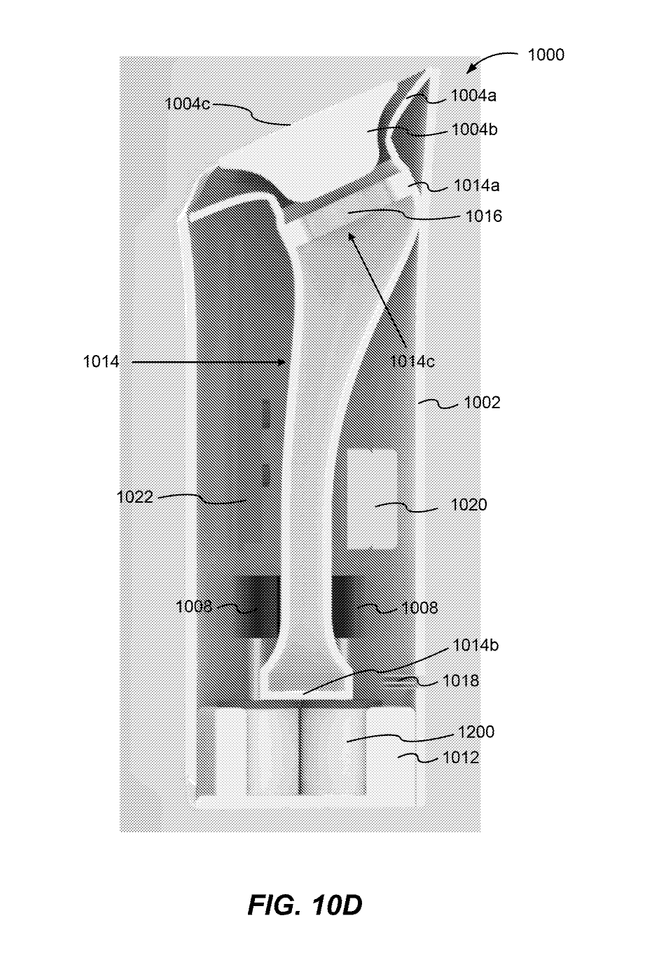

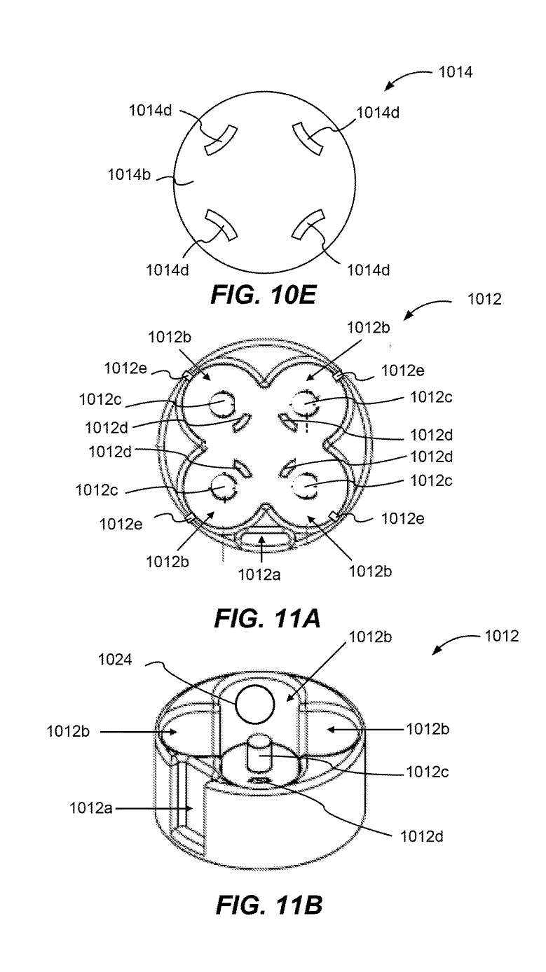

A scent dispensing device 1000 according to one illustrated embodiment will now be described with reference to FIGS. 10A-10E. FIG. 10A is an isometric view of the scent dispensing device 1000. FIGS. 10B and 10C are partially exploded views of the scent dispensing device 1000. FIG. 10D is cross-sectional view taken along line II-II of FIG. 10A. FIG. 10E is a bottom plan view of a scent mixing chamber shown in FIG. 10D.

As shown in FIG. 10A, the scent dispensing device 1000 includes a housing 1002 having a recessed portion 1002a, a diffuser 1004, and a switch 1006. As shown in FIG. 10B, the scent dispensing device 1000 also includes four motors 1008, four scent cartridges 1200, and a scent cartridge holder 1012. Each of the motors 1008 includes a housing 1008a, a shaft 1008b, and an end portion 1008c. In one embodiment, each of the motors 1008 is a stepper motor. As shown in FIG. 10C, the scent cartridge holder 1012 has a recessed portion 1012a that can be aligned with the recessed portion 1002a of the housing 1002. The recessed portions 1002a and 1012a facilitate removal of the scent cartridge holder 1012 from the housing 1002 so that cartridges 1200 can be loaded into and/or unloaded from the scent cartridge holder 1012. The recessed portions 1002a and 1010a also serve as alignment structures that ensure that the scent cartridge holder 1012 has a predetermined orientation relative to the housing 1002 when the scent cartridge holder 1012 is disposed within the housing 1002.

As shown in FIG. 10D, the diffuser 1004 includes a first diffuser portion 1004a, a second diffuser portion 1004b, and a third diffuser portion 1004c. The first diffuser portion 1004a, the diffuser second portion 1004b, and the third diffuser portion 1004c are similar to the first diffuser portion 134, the second diffuser portion 136, and the third diffuser portion 138, respectively. The scent dispensing device 1000 includes a scent mixing chamber 1014 that includes an upper portion 1014a and a lower portion 1014b. The upper portion 1014a of the scent mixing chamber 1014 includes an aperture 1014c that serves as an outlet port for the scent mixing chamber 1014. A fan 1016 is disposed in the aperture 1014c of the scent mixing chamber 1014. The scent dispensing device 1000 also includes an interface connector 1018, one or more batteries 1020, and a printed circuit board 1022. In one embodiment, the communications interface connector 1018 is a USB A-Type, female connector. The printed circuit board 1022 includes various components and circuits for communicating and processing scent information, and for controlling operation of the motors 1008 and the fan 1016 based on such scent information.

As shown in FIG. 10E, the lower portion 1014b of the scent mixing chamber 1014 includes four wedge-shaped apertures 1014d. Each of the wedge-shaped apertures 1014d serves as an inlet port to the scent mixing chamber 1014.

FIG. 11A is a top plan view of the scent cartridge holder 1012 shown in FIG. 10B. FIG. 11B is an isometric view of the scent cartridge holder 1012. The scent cartridge holder 1012 includes four scent cartridge receptacles 1012b, each of which is sized and dimensioned to receive at least a lower portion of one of the scent cartridges 1200. Each scent cartridge receptacle 1012b includes a shaft or axle 1012c, a wedge-shaped aperture 1012d, and an alignment indicator 1012e. The alignment indicator 1012e may take a variety of forms, for example, a marking, an indentation, or a projection.

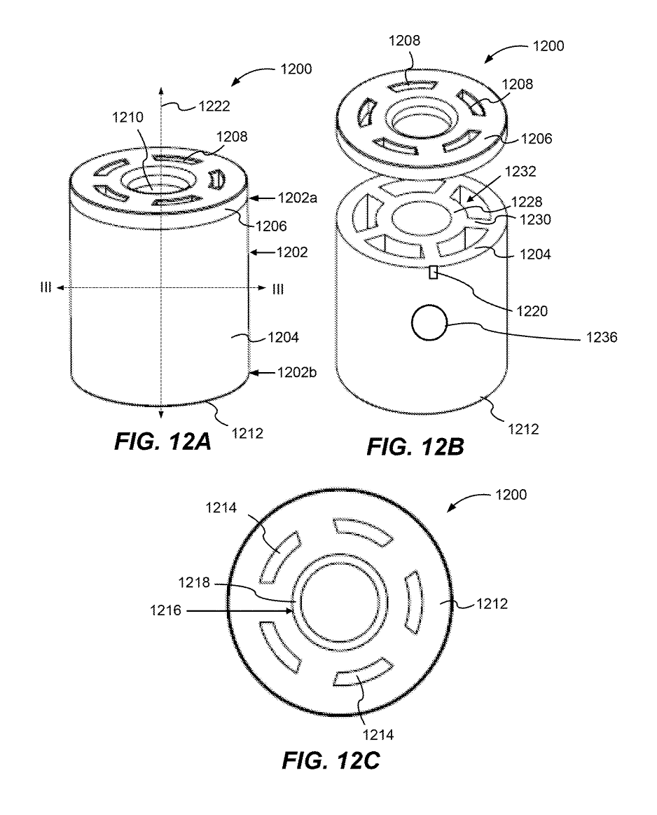

The scent cartridge 1200 according to one illustrated embodiment will now be described with reference to FIGS. 12A-12C. FIG. 12A is an isometric view of the scent cartridge 1200. FIG. 12B is a partially exploded view of the scent cartridge 1200. FIG. 12C is a bottom plan view of the scent cartridge 1200. As shown in FIG. 12A, the scent cartridge 1200 includes a housing 1202 having a circular peripheral perimeter. The housing 1202 includes a first end 1202a and a second end 1202b. A cylindrical outer wall 1204 defines a peripheral perimeter of the housing 1202.

A first end portion 1206 is disposed at the first end 1202a of the housing 1202. The first end portion 1206 includes five wedge-shaped apertures 1208 and an aperture 1210. The aperture 1210 and the end portion 1008c of the shaft 1008b of each of the motors 1008 are sized and dimensioned such that the end portion 1008c can be inserted into the aperture 1210. In one embodiment, the end portion 1008c and the aperture 1210 are circular and the end portion 1008c can be inserted snugly into the aperture 1210 such that rotation of the shaft 1008b causes rotation of the scent cartridge 1200 about a longitudinal, rotational axis 1222. In one embodiment, the end portion 1008c and the aperture 1210 are “D” shaped and the end portion 1008c of the shaft 1008b of each motor 1008 can be inserted into the aperture 1210 such that rotation of the shaft 1008b causes rotation of the scent cartridge 1200 about the longitudinal axis 1222.

A second end portion 1212 is disposed at the second end 1202b of the housing 1202. The second end portion 1212 includes five wedge-shaped apertures 1214. A circular aperture 1216 is formed in the second end portion 1212 and extends upwardly into the housing 1202 and ends a bearing surface 1218. The circular aperture 1216 and the axles 1012c of the scent cartridge holder 1012 are sized and dimensioned such that each axle 1012c can be inserted into the circular aperture 1216 such that the scent cartridge 1200 is able to rotate about the longitudinal axis 1222 when the axle 1012c is inserted into the circular aperture 1216. An annular bearing surface 1018 is included at the second end 1202b of the housing 1200 adjacent the circular aperture 1216. The annular bearing surface 1018 is disposed about the axis of rotation 1222 such that, when the axle 1012c of the scent cartridge holder 1012 is inserted into the circular aperture 1216 and the scent cartridge 1200 is rotated, the annular bearing surface 1018 abuts the side surfaces of the axle 1012c.

As shown in FIG. 12B, a plurality of walls extends upwardly from the second end portion 1212, including the outer wall 1204, an axial wall 1228, and five planar walls 1230. The planar walls 1230 extend radially from the axial wall 1228 to the outer wall 1204. The outer wall 1204, the first end portion 1206, the second end portion 1212, the axial wall 1228, and the planar walls 1230 define five chambers 1232 within the scent cartridge 1200 that are annularly arrayed about the rotational axis 1222. For each of the chambers 1232, one of the apertures 1208 formed in the first end portion 1206 is aligned with a corresponding aperture 1214 formed in the second end portion 1212. Each chamber 1232 defines a channel in which air can be fluidly communicated from the aperture 1214 formed in the second end portion 1212 to the corresponding aperture 1208 formed in the first end portion 1206. The outer wall 1204, the axial wall 1228, and the planar walls 1230 prevent air flowing through one of the chambers 1232 from leaking into any other of the chambers 1232.

As will be explained in detail below, the end portion 1008c of the shaft 1008b of the motors 1008 can engage the aperture 1216 of the scent cartridge 1200 and cause it to rotate into one of five predetermined positions/orientations in which a particular one of the wedge-shaped apertures 1208 of the first end portion 1206 of the scent cartridge 1200 is aligned with one of the wedge-shaped apertures 1014d of the lower portion 1014b of the scent mixing chamber 1014. When the scent cartridge 1200 is in such a position, the corresponding wedge-shaped aperture 1214 of the second end portion 1212 of the scent cartridge 1200 is aligned with one of the wedge-shaped apertures 1012d of the scent cartridge holder 1012. In one embodiment, the size and dimensions of the wedge-shaped apertures 1012d, 1014c, 1208, and 1214 are the same. When the scent cartridge 1200 is in such a position/orientation, the fan 1016 can cause air to flow into one of the wedge-shaped apertures 1012d of the scent cartridge holder 1012, through one of the chambers 1232 via corresponding wedge-shaped apertures 1214 and 1208, through the scent mixing chamber 1014 via apertures 1014d and 1014c, and out of the diffuser 1004.

The scent cartridge 1200 also includes an alignment indicator 1220. The alignment indicator 1220 may take a variety of forms, for example, a marking, an indentation, or a projection. When the alignment indicator 1220 of one of the scent cartridges 1200 is aligned with one of the alignment indicators 1012e of the scent cartridge holder 1012, an aperture 1214 of a predetermined one of the chambers 1232 (i.e., a default chamber) is aligned with one of the apertures 1012d formed in the scent cartridge holder 1012. A user of the scent dispensing device 1000 may align the alignment indicator 1220 of one of the scent cartridges 1200 with one of the alignment indicators 1012e of the scent cartridge holder 1012 when the scent cartridge 1200 is initially installed in the scent cartridge holder 1012. When the alignment indicator 1220 of one of the scent cartridges 1200 is aligned with one of the alignment indicators 1012e of the scent cartridge holder 1012, and the scent cartridge holder 1012 is inserted into the housing 1002 of the scent dispensing device 1000, an aperture 1208 of the first end portion 1206 of the default chamber 1232 of the scent cartridge 1200 is aligned with one of the apertures 1014d of the lower portion 1014b of the scent mixing chamber 1014.

Although the scent cartridge 1200 is described in connection with the scent dispensing device 1000, the scent cartridge 1200 may be used in other scent dispensing devices. For example, the scent cartridge 1200 may be used in other scent dispensing devices that are included with printers, cameras, clothing, chairs, kitchen appliances, and automobiles. Such scent dispensing devices may enable a user to specify a particular scent or scent combination by manually rotating one or more of the scent cartridges 1200 such that a particular chamber 1232 of each of the scent cartridges 1200 has a predetermined orientation. Alternatively, the scent dispensing device 1000 may be integrated with other devices, such as printers, cameras, clothing, chairs, kitchen appliances, and automobiles.

The scent cartridge holder 1012 of the scent dispensing device 1000 described above includes four scent cartridge receptacles 1012b, and each of the scent cartridges 1200 described above includes five chambers 1232. If four of the chambers 1232 of each of the scent cartridges 1200 include a scent medium 1234 (e.g., one of the chambers 1232 of each of the scent cartridges 1200 is empty) and the scent of each scent medium 1234 is different than that of every other scent medium 1234, the scent dispensing device 1000 could dispense 340 different scent combinations (i.e., 4⁴+4³+4−²+4¹).

In one embodiment, the scent cartridge holder 1012 of the scent dispensing device 1000 includes five scent cartridge receptacles 1012b, and each of the scent cartridges 1200 includes six chambers 1232. If five of the chambers 1232 of each of the scent cartridges 1200 include a scent medium 1234 (e.g., one of the chambers 1232 of each of the scent cartridges 1200 includes an air filter) and the scent of each scent medium 1234 is different than that of every other scent medium 1234, the scent dispensing device 1000 could dispense 3,905 different scent combinations (i.e., 5⁵+5−⁴+5³+5²+5¹).

In another embodiment, the scent cartridge holder 1012 of the scent dispensing device 1000 includes six scent cartridge receptacles 1012b, and each of the scent cartridges 1200 includes seven chambers 1232. If six of the chambers 1232 of each of the scent cartridges 1200 include a scent medium 1234 (e.g., one of the chambers 1232 of each of the scent cartridges 1200 is empty) and the scent of each scent medium 1234 is different than that of every other scent medium 1234, the scent dispensing device 1000 could dispense 55,986 different scent combinations (i.e., 6⁶+6−⁵+6⁴+6³+6²+6¹).

Of course, the scent cartridge holder 1012 of the scent dispensing device 1000 may include another number of scent cartridge receptacles 1012b, and each of the scent cartridges 1200 may include another number of chambers 1232. Additionally, the scent media 1234 can be distributed within the chambers 1232 of the scent cartridges 1200 such that the scent dispensing device 1000 could produce virtually any desired number of different scent combinations.

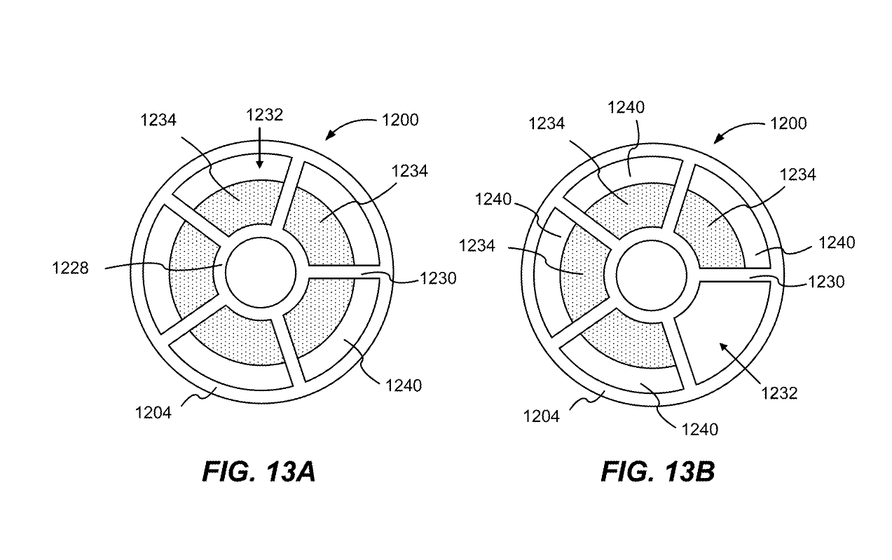

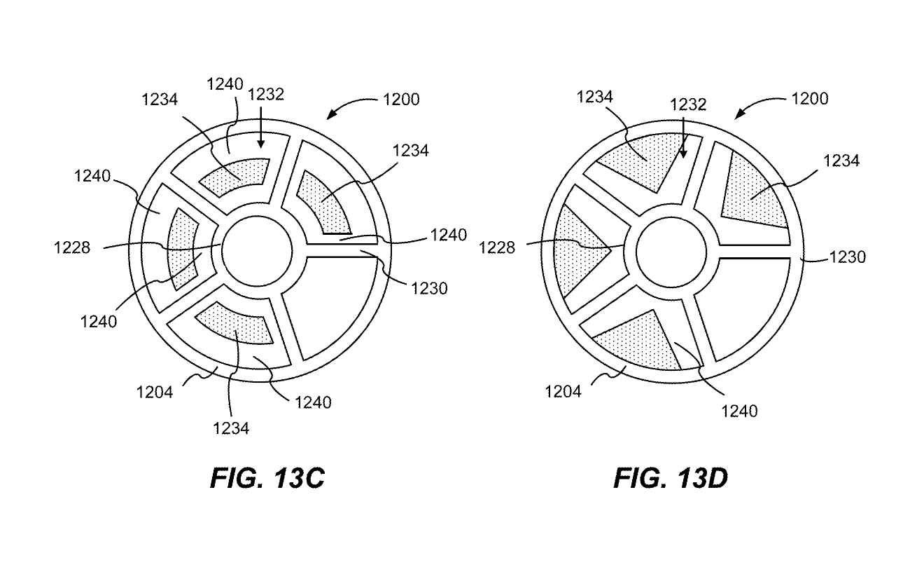

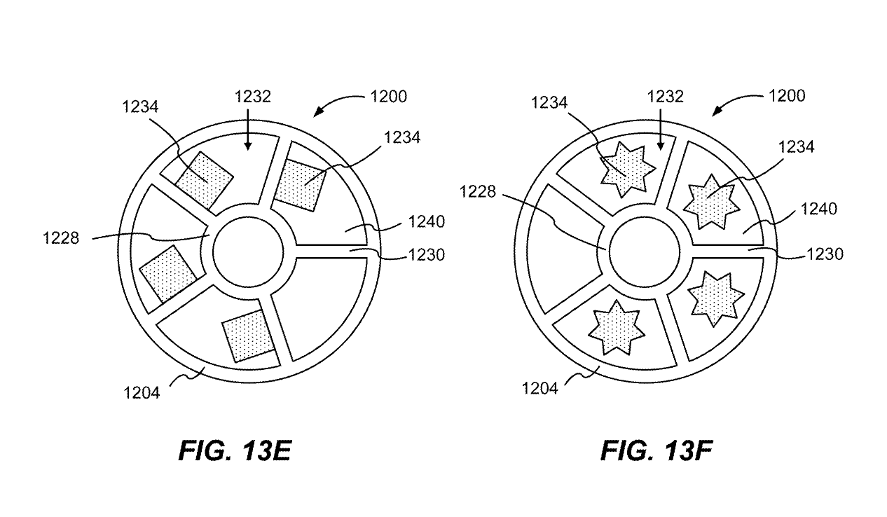

A number of example embodiments of the scent cartridge 1200 will now be described with reference to FIGS. 13A-13F. Each of FIGS. 13A-13F shows a horizontal slice view of the scent cartridge 1200 along the line III-III shown in FIG. 12A. In each of the example embodiments, one or more scent media 1234 are arranged in one or more of the chambers 1232. Each scent medium 1234 is arranged similarly to the scent medium 934 described above in connection with FIG. 9B. For example, each scent medium 1234 may be arranged in one of the chambers 1232 between corresponding ones of the apertures 1208 and 1214 such that a first selective membrane 936 is disposed between the scent medium 1234 and the aperture 1208 and/or a second selective membrane 938 is disposed between the scent medium 1234 and the aperture 1214.

FIG. 13A illustrates an embodiment in which each of the chambers 1232 includes a scent medium 1234. One or more of the scent media 1234 may have a scent or aroma that is the same as or different than that of one or more of the other scent media 1234. For example, each scent medium 1234 may have a scent or aroma that is different than that of the other scent media 1234. One or more of the scent media 1234 may be unscented, for example, an air filter medium (e.g., a medium including a charcoal filter) and/or a scent neutralizing medium (e.g., a medium including baking soda).

As shown in FIG. 13B, one or more of the chambers 1232 may be empty. Additionally, a gap 1240 may be disposed between each of the scent media 1234 and one or more of the walls that define each chamber 1232. For example, within one or more of the chambers 1232, the gap 1240 may be disposed between the scent medium 1234 and the outer wall 1204.

As shown in FIG. 13C, within one or more of the chambers 1232, the gap 1240 may be disposed between the scent medium 1234 and each of the walls that define the one or more chambers 1232. That is, within one or more chambers 1232, the gap 1240 may be disposed between the scent medium 1234 and the outer wall 1204, the axial wall 1228, and a pair of planar walls 1230.

The scent media 1234 may be disposed on one of the walls of one or more of the chambers 1232. For example, each scent medium 1234 may be disposed on the outer wall 1204 as shown in FIG. 13D, or may be disposed on one of the planar walls 1230 as shown in FIG. 13E.

Each scent medium 1234 may be arranged such that greater than fifty percent of the total surface area of the scent medium 1234 is exposed to the air that flows through one of the chambers 1232. For example, the dimensions of each scent medium 1234 shown in FIG. 13E may be as follows: the length is equal to 1 centimeter (cm), the width is equal to 1 cm, and the height is 5 cm. Accordingly, the total surface area of each scent medium 1234 is the surface area of four side portions (i.e., 4×1 cm×5 cm=20 cm²) plus the surface area of two end portions (i.e., 2×1 cm×1 cm=2 cm²) or 22 cm². The surface area of the side portion touching one of the planar walls 1230 is 1 cm×5 cm or 5 cm². Assuming that the apertures 1208 and 1214 of the first and the second end portions 1206 and 1212, respectively, are sized and dimensioned such that one-hundred percent of the surface area of each end portion of the scent media 1234 is exposed to air flowing in each chamber 1232, the surface area of each scent medium 1234 that is exposed to air flowing through one of the chambers 1232 is the surface area of three side portions (i.e., 3×1 cm×5 cm=15 cm²) plus the surface area of two end portions (i.e., 2×1 cm×1 cm=2 cm²) or 17 cm². Accordingly, roughly 72% (i.e., 17 cm²/22 cm²) of each scent medium 1234 is exposed to air flowing through one of the chambers 1232.

FIG. 13F shows an example in which a star-shaped scent medium 1234 is provided in four of the chambers 1232, such that the gap 1240 is provided between the scent media 1234 and inner surfaces of each of the walls that define the chambers 1232. Assuming that the apertures 1208 and 1214 of the first and the second end portions 1206 and 1212, respectively, are sized and dimensioned such that one-hundred percent of the surface area of each end portion of the scent media 1234 is exposed to air flowing in the chambers 1232, one-hundred percent of the surface area of each scent medium 1234 is exposed to air flowing through one of the chambers 1232.

The example cross-sectional shapes and positions of the scent media 1234 shown in FIGS. 13A-13F are illustrative and are not limiting. The cross-sectional shapes of the scent media 1234 may take a variety of shapes including circles, squares, rectangles, triangles, ovals, wedges, stars, etc. Additionally, each scent medium 1234 may be arranged such that the gap 1240 is provided between the scent medium 1234 and an inner surface of at least one wall of the one of the chambers 1232, wherein the gap 1240 extends along the length of the housing 1202.

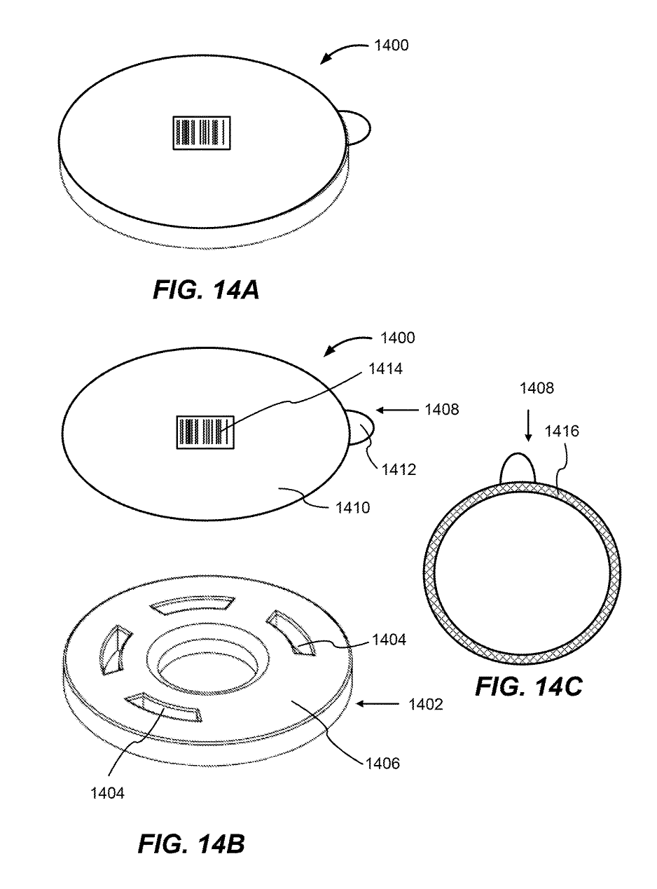

FIG. 14A is an isometric view of the top of a scent cartridge 1400, according to one illustrated embodiment. FIG. 14B is an exploded view of the top of the scent cartridge 1400. The scent cartridge 1400 is similar to the scent cartridge 1200 shown in FIGS. 12A-12C. The scent cartridge 1400 includes an end portion 1402 having four wedge-shaped apertures 1404 and a sealed portion 1406, which is provided between two of the apertures 1404. The surface area of the sealed portion 1406 may be less than, greater than, or equal to the surface area of each of the apertures 1404.

The scent cartridge 1400 also includes one or more hermetically sealed membranes 1408. The hermetically sealed membrane 1408 may be formed from tin foil, aluminum foil, or a foil formed from an alloy thereof, for example. The hermetically sealed membrane 1408 includes a main portion 1410, a tab 1412, and a contents indicator 1414 formed on an upper surface of the main portion 1410.

The contents indicator 1414 may include a picture, text, and/or a computer-readable structure (e.g., a barcode or RFID tag) indicating the particular scents, aromas, or other items contained in each of the chambers of the scent cartridge 1400. For example, the contents indicator 1414 may indicate that a first specified chamber includes particles having a first scent (e.g., the scent of hazelnut coffee), a second specified chamber includes particles having a second scent (e.g., the scent of espresso), a third specified chamber includes particles having a third scent (e.g., the scent of Kona coffee), that a forth specified chamber includes particles that neutralize odors (e.g., baking soda), and that a fifth specified chamber is empty. In one embodiment, the contents indicator 1414 is provided on a housing of the cartridge 1400.

FIG. 14C shows a bottom plan view of the hermetically sealed membrane 1408. A pressure sensitive adhesive 1416 is provided about a peripheral portion of a lower surface of the hermetically sealed membrane 1408. The hermetically sealed membrane 1408 may be placed above the first end portion 1402 and advanced toward the first end portion 1402 until the pressure sensitive adhesive 1416 contacts an upper surface of the first end portion 1402. Pressure may be applied to the upper surface of the main portion 1410 of the hermetically sealed membrane 1408 such that the main portion 1410 forms a seal around the apertures 1404 and prevents air and moisture from flowing from through the apertures 1404, which can degrade the scent media included in chambers of the scent cartridge 1400. The hermetically sealed membrane 1408 may be removed from the first end portion 1402 prior to use of the cartridge 1400, and may be reattached to the first end portion 1402 after use of the cartridge 1400 to protect the scent media therein. The cartridge 1400 also may include a second hermetically sealed membrane 1408 that can be attached to a second end portion of the cartridge 1400 that is similar to the second end portion 1212 shown in FIG. 12C.

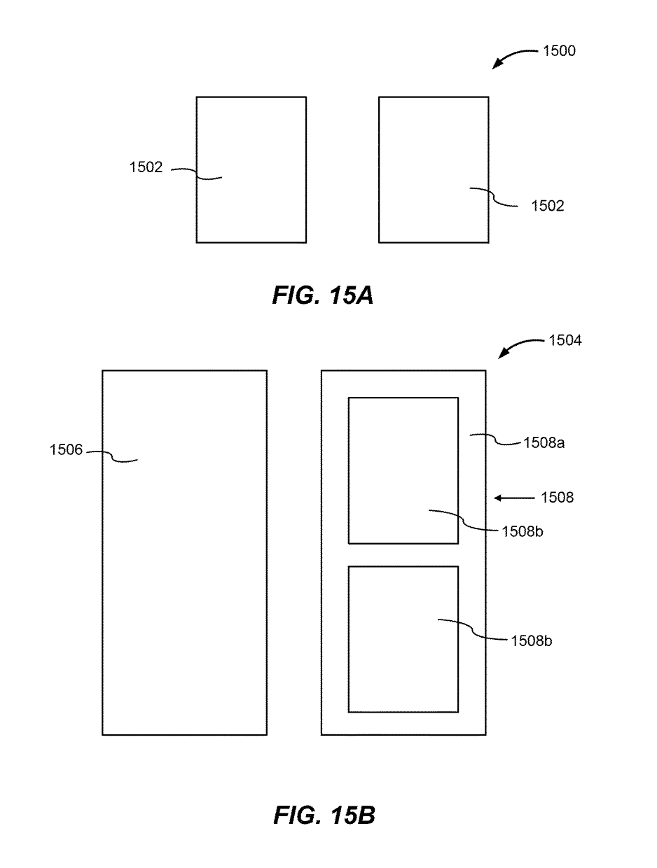

FIG. 15A is an exploded view of a pouch 1500 according to one illustrated embodiment. The pouch 1500 includes one or more side portions 1502. In one embodiment, the pouch 1500 includes two side portions 1502 formed of a plastic material that does not enable air and moisture to pass therethrough. One or more scent cartridges (e.g., cartridge 1200) are placed between the side portions 1502, and corresponding edges of the side portions 1502 are welded together such that an air tight and moisture proof pouch is formed around the cartridge, which protects the contents of the cartridge prior to use of the cartridge. In one embodiment, one side portion 1502 is provided, which is folded around one or more scent cartridges, and corresponding edges of the side portion 1502 are welded together to form an air tight and moisture proof pouch around the cartridges. In one embodiment, the pouch 1500 includes a desiccant (e.g., calcium oxide or silica gel) that can absorb moisture that may be contained in the pouch 1500 after the pouch 1500 is sealed.

FIG. 15B is an exploded view of a scent cartridge blister pack 1504 according to one illustrated embodiment. The blister pack 1504 includes a backing substrate 1506 and a blister substrate 1508. The blister substrate 1508 includes a border portion 1508a and one or more compartments 1508b that extend outwardly from a first surface of the blister substrate 1508. After a scent cartridge is placed in each of the compartments 1508b, an adhesive is applied to the border portion 1508a on a second surface opposite the first surface of the blister substrate 1508, and the backing substrate 1506 is brought into contact with the adhesive on the border portion 1508a. The backing substrate 1506 hermetically seals each of the compartments 1508b, thereby protecting the contents of the cartridges sealed therein from external air and moisture. In one embodiment, each of the compartments 1508b includes a desiccant (e.g., calcium oxide or silica gel) that can absorb moisture that may be contained in the compartments 1508b after the compartments 1508b are sealed.

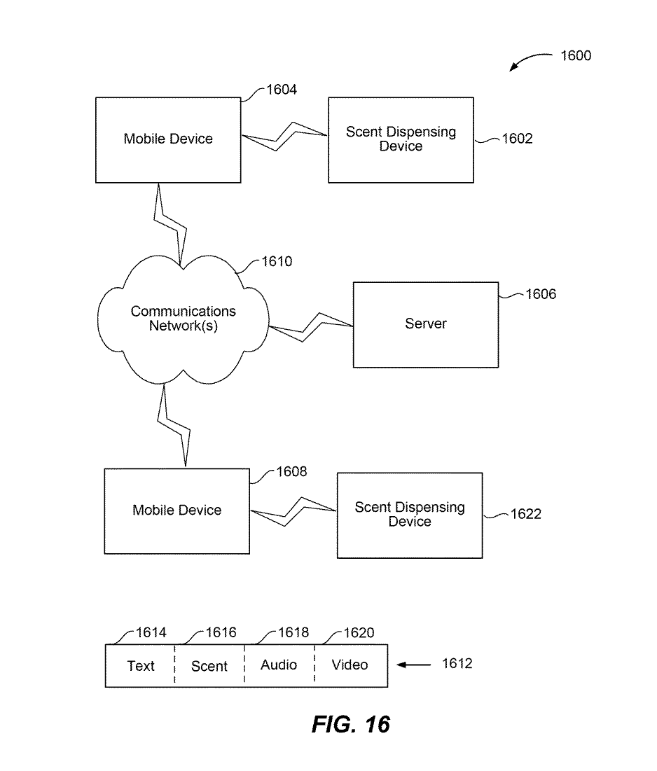

FIG. 16 is a network diagram of a scent dispensing system 1600 according to one illustrated embodiment. The scent dispensing system 1600 includes a scent dispensing device 1602, a first mobile device 1604, a server 1606, and a second mobile device 1608. One or more communications networks 1610 provide communications paths between the first mobile device 1604, the server 1606, and the second mobile device 1608. The scent dispensing device 1602 and the first mobile device 1604 communicate via wired or wireless communications. In one embodiment, the scent dispensing device 1602 and the first mobile device 1604 communicate via Bluetooth communications.

As will be explained below, the first mobile device 1604, the server 1606, and/or the second mobile device 1608 may include computer-executable code that, when executed by a processor, generates one or more messages 1612. Each message 1612 may include a text portion 1614, a scent portion 1616, an audio portion 1618, and/or a video portion 1620. The first mobile device 1604 stores a software application including computer-executable instructions that, when executed by a processor, cause the first mobile device 1604 to process the messages 1612. For example, the first mobile device 1604 may begin to execute such instructions when one or more of the messages 1612 are received via one or more applications, such as an electronic mail application and/or a social media application (e.g., Twitter or Facebook).

Upon receipt of one or more of the messages 1612 the software application may, for example, instruct the processor to cause a display device (e.g., a touch screen) of the first mobile device 1604 to display the text portion 1614, which may include a description of the scent portion 1616, the audio portion 1618, and/or the video portion 1620 of the message 1612. A user of the first mobile device 1604 may indicate using an input device (e.g., a key pad or touch screen) whether the message 1612 or a sequence of related messages 1612 may be processed further. If the processor receives an indication that the message(s) 1612 are to be processed further, the processor may cause the first mobile device 1604 to send information corresponding to the scent portion 1616 to the scent dispensing device 1602. The processor also may cause a speaker to produce one or more sound(s) corresponding to the audio portion 1618 and/or a display device to display one or more image(s) corresponding to the video portion 1620.

Additionally, one or more software applications may include computer-executable instructions for gathering information regarding the contents of scent cartridges (e.g., scent cartridges 900, 1200, or 1400). For example, the software application may enable a user to indicate that one or more scent cartridges are being or have been loaded into the scent dispensing device 1602. In response, the software application instructs the processor to cause a display device (e.g., a tough screen) of the first mobile device 1604 to display a series of prompts for a user to input information regarding each cartridge that has been or is being loaded into the scent dispensing device 1602. Such information may include information regarding a particular location within the scent dispensing device 1602 (e.g., a particular scent cartridge receptacle 1012b of a scent cartridge holder 1012) and information regarding the contents of each scent chamber (e.g., an identifier of each scent chamber 1232 and an identifier of the contents thereof). For example, the user may respond to the prompts indicating that the front, right scent cartridge receptacle 1012b has been loaded with a scent cartridge that includes five chambers, wherein a first predefined chamber contains a first scent, a second predefined chamber contains a second scent, a third predefined chamber contains a third scent, a fourth predefined chamber contains an odor neutralizer (e.g., baking soda), and a fifth predefined chamber is empty.

Additionally or alternatively, the software application may cause the display device of the first mobile device 1604 to display a message instructing the user to place a barcode included with the scent cartridge within the field of view of a camera of the first mobile device. The software application may then instruct the processor to cause the camera to take an image and to process corresponding image data to obtain the information regarding the contents of the cartridge. In this case, the user may simply enter information regarding the location within the scent dispensing device 1602 (e.g., a particular scent cartridge receptacle 1012b of a scent cartridge holder 1012) in which the cartridge is being or has been placed. After the software application has caused the processor to store information regarding the contents and locations of the cartridges loaded within the scent dispensing device 1602 into a memory of the first mobile device 1604, the application can process the messages 1612.

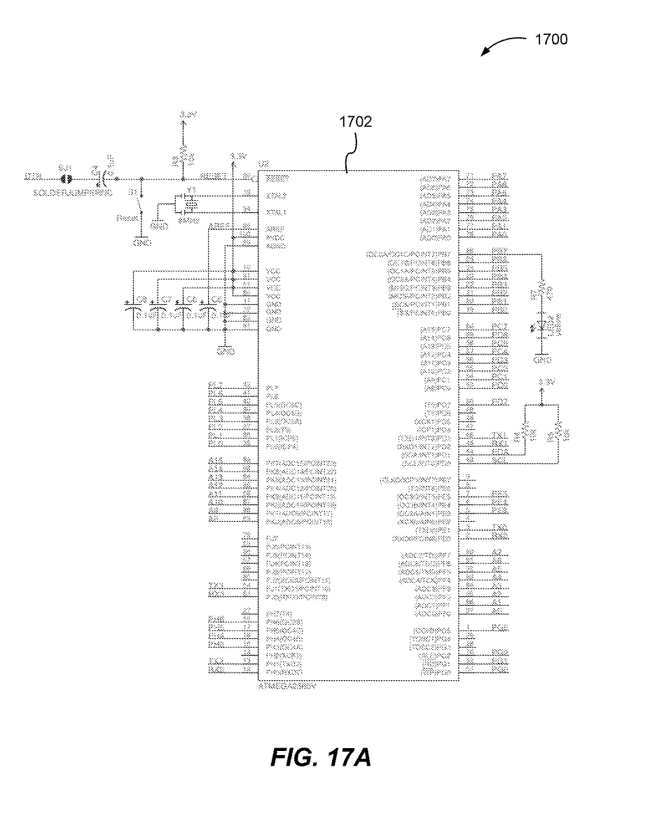

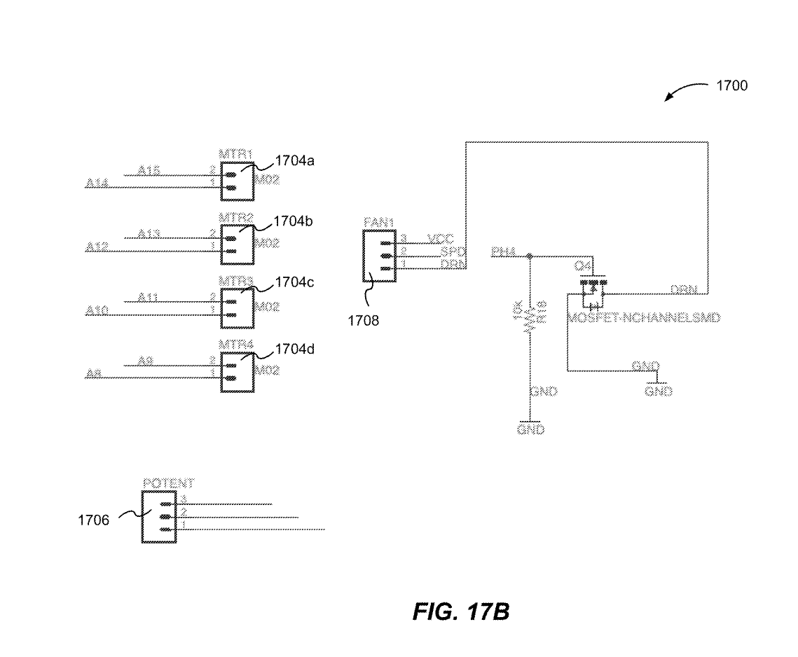

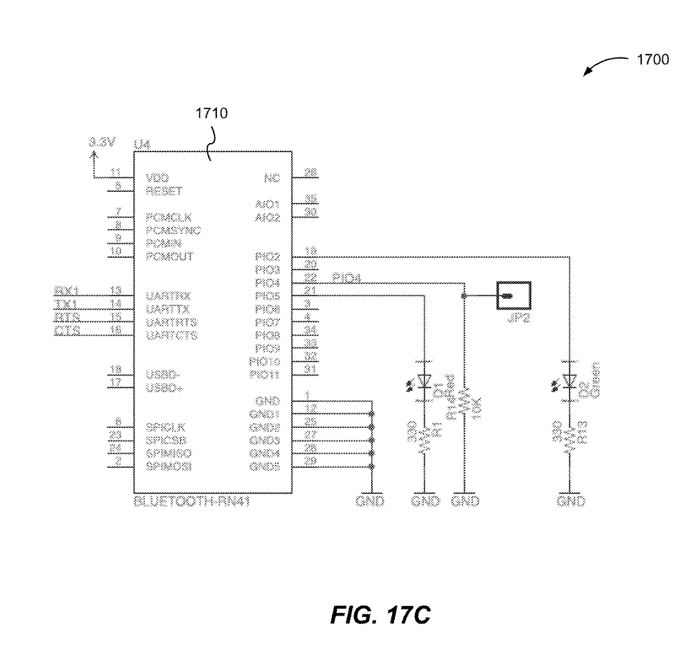

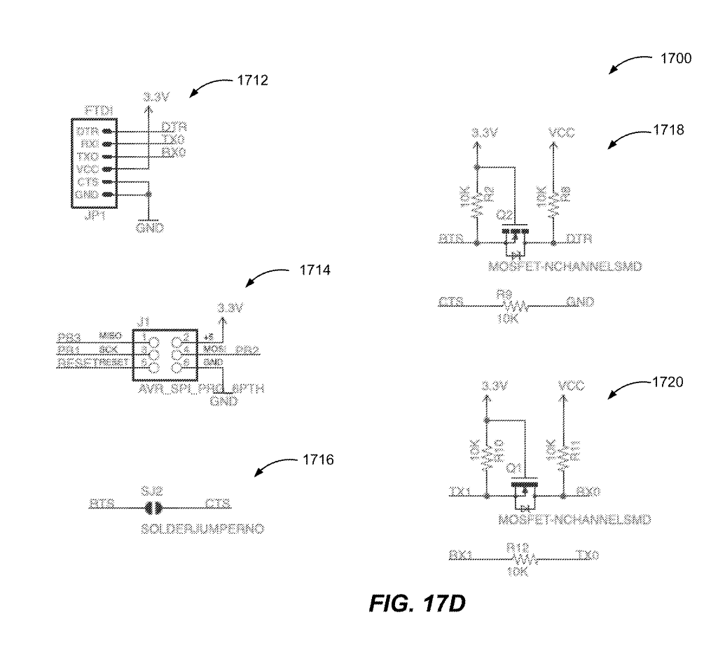

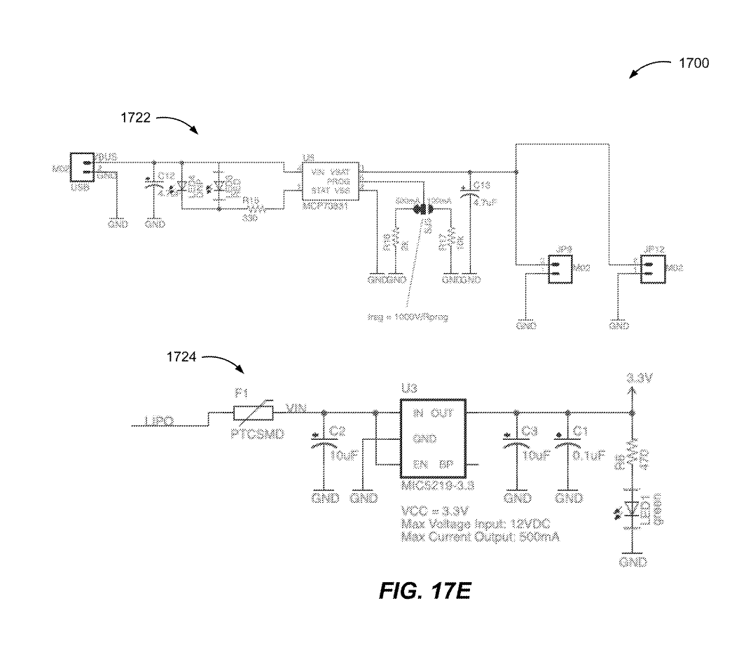

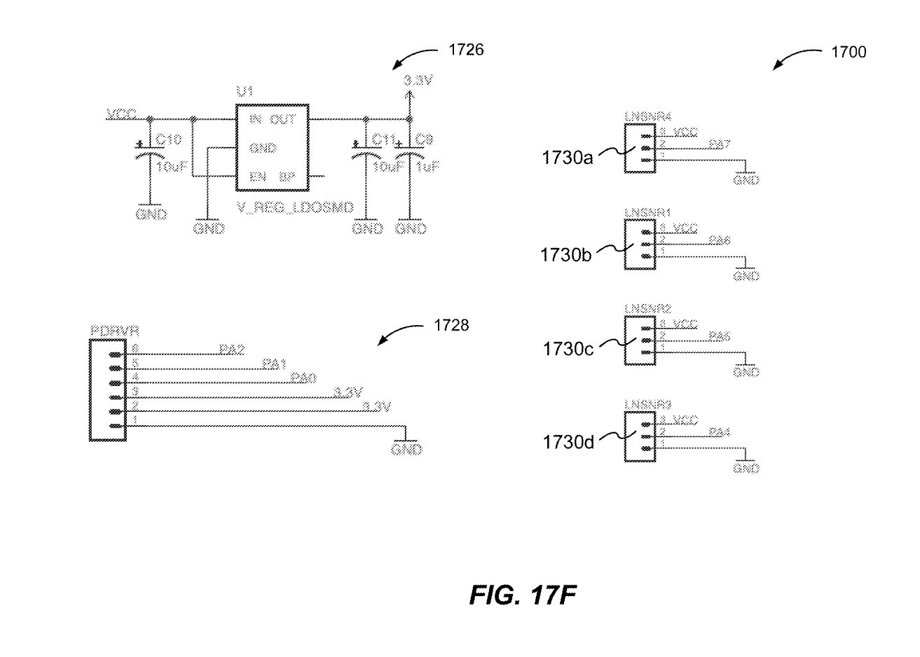

FIGS. 17A-17F illustrate various portions of a schematic diagram of a computing device 1700 according to one illustrated embodiment. The computing device 1700 may be used to implement the scent dispensing device 1602 shown in FIG. 16. The computing device 1700 includes a microcontroller 1702, four stepper motors 1704a-1704d (collectively 1704), a slide potentiometer 1706, a fan 1708, and a Bluetooth communications module 1710. In one embodiment, the microcontroller 1702 is a model ATMEGA2560V microcontroller by Atmel Corporation. The computing device 1700 also includes circuits 1712, 1714, 1716, 1718, 1720, 1722, 1724, 1726, and 1728, which perform various functions such as regulating power, charging batteries, monitoring battery levels, distributing power, biasing components, and processing USB communications. Additionally, the computing device 1700 may include four sensors 1730a-1730d (e.g., cameras) that can provide image data that the microcontroller 1702 can process to determine one or more orientations/positions of one or more scent cartridges.

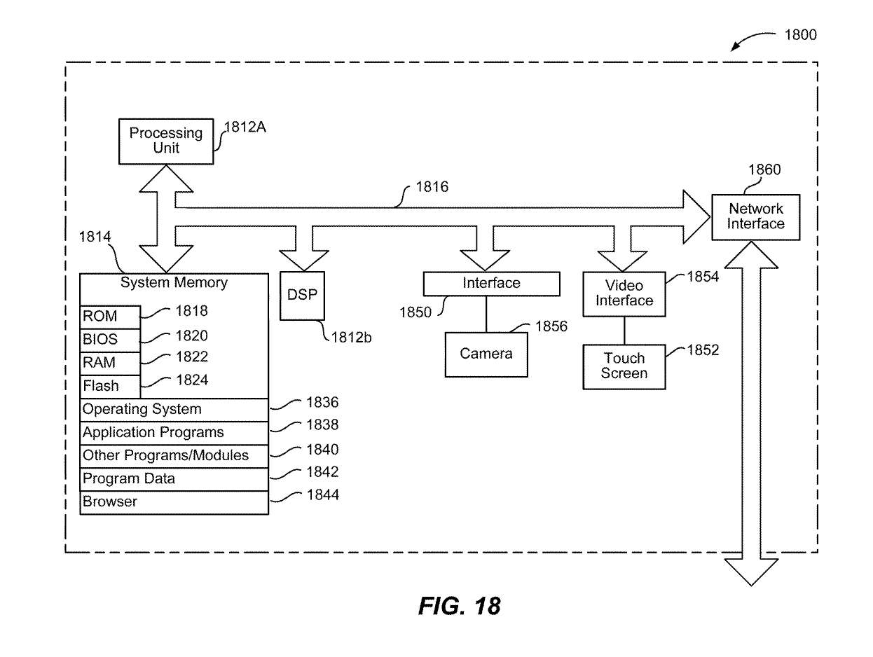

FIG. 18 is a block diagram of a computer system 1800 according to one illustrated embodiment. The computer system 1800 may be used to implement the first mobile device 1604 and/or the second mobile device 1608 shown in FIG. 16. The computer system 1800 is suitable for facilitating generation and processing of scent messages (e.g., message 1612). The computer system 1800 may include one or more processing units 1812a and 1812b (collectively processing unit 1812), a system memory 1814 and a system bus 1816 that couples various system components including the system memory 1814 to the processing units 1812. The processing unit 1812 may be any logic processing unit, such as one or more central processing units (CPUs) 1812a, digital signal processors (DSP) 1812b, application-specific integrated circuits (ASICs), field programmable gate arrays (FPGAs), etc. The system bus 1816 can employ any known bus structures or architectures, including a memory bus with memory controller, a peripheral bus, and a local bus. The system memory 1814 includes read-only memory (ROM) 1818, random access memory (RAM) 1822, and flash memory 1824. A basic input/output system (BIOS) 1820, which can form part of the ROM 1818, contains basic routines that help transfer information between elements within the computer system 1800, such as during start-up.

The computer system 1800 may also include a plurality of interfaces such as network interface 1860 and interface 1850 supporting any other wireless/wired interfaces. Computer-readable storage media can be used to store the information that may be accessed by processing unit 1812a. For example, such computer-readable storage media may include, but is not limited to, random access memory (RAM), read-only memory (ROM), electrically erasable programmable read-only memory (EEPROM), flash memory, or other solid state memory or any other medium.

Program modules can be stored in the system memory 1814, such as an operating system 1836, one or more application programs 1838, other programs or modules 1840, and program data 1842. Application programs 1838 may include instructions that cause the processor unit(s) 1812 to generate, process, and/or receive scent messages and scent information. Other program modules 1840 may include instructions for handling security such as password or other access protection and communications encryption. The system memory 1814 may also include communications programs, for example, a Web client or browser 1844 for permitting the computer system 1800 to access and exchange data with sources such as Web sites of the Internet, corporate intranets, extranets, or other networks and devices as described herein, as well as other server applications on server computing systems. The browser 1844 in the depicted embodiment is markup language based, such as Hypertext Markup Language (HTML), Extensible Markup Language (XML) or Wireless Markup Language (WML), and operates with markup languages that use syntactically delimited characters added to the data of a document to represent the structure of the document. A number of Web clients or browsers are commercially available such as those from Mozilla, Google, and Microsoft.

An operator can enter commands and information into the computer system 1800 through input devices such as a touch screen 1852, and/or via a graphical user interface. Other input devices can include a microphone, a pointing device, etc. These and other input devices, such as camera unit 1856, are connected to one or more of the processing units 1812 through the interface 1850 such as a serial port interface that couples to the system bus 1816, although other interfaces such as a parallel port, a game port or a wireless interface or a universal serial bus (USB) can be used. The touch screen device 1852 or other display device is coupled to the system bus 1816 via a video interface 1854, such as a video adapter.

The computer system 1800 can operate in a networked environment using logical connections to one or more remote computers and/or devices as described above with reference to FIG. 16. For example, the computer system 1800 can operate in a networked environment using logical connections to one or more mobile devices, landline telephones and other service providers or information servers. Communications may be via a wired and/or wireless network architecture, for instance wired and wireless enterprise-wide computer networks, intranets, extranets, telecommunications networks, cellular networks, paging networks, and other mobile networks.

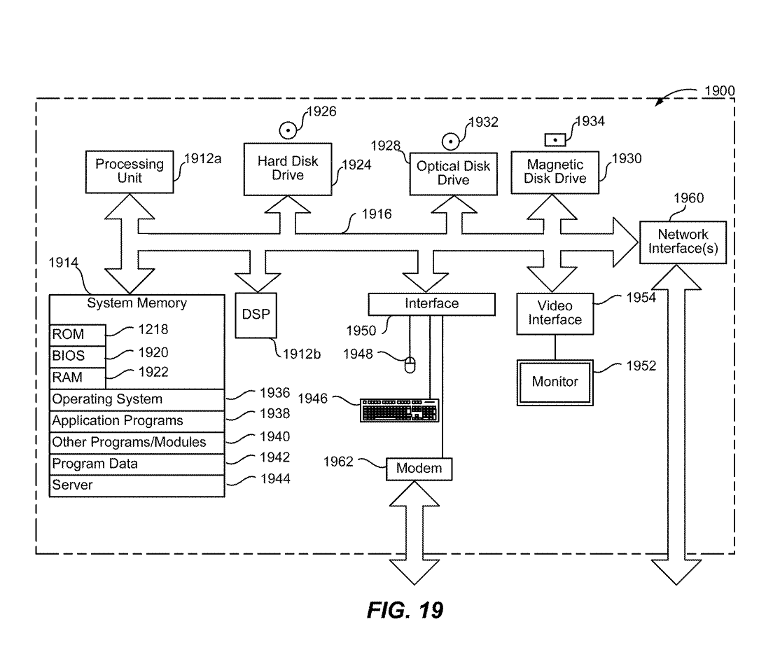

FIG. 19 shows an example computer system 1900, according to one illustrated embodiment. The computer system 1900 may be used to implement the server 1606 shown in FIG. 16. The computer system 1900 will at times be referred to in the singular herein, but this is not intended to limit the embodiments to a single device, since in typical embodiments, there may be more than one computer system or device involved. Unless described otherwise, the construction and operation of the various blocks shown in FIG. 19 are of conventional design. As a result, such blocks need not be described in further detail herein, as they will be understood by those skilled in the relevant art.

The computer system 1900 may include one or more processing units 1912a, 1912b (collectively 1912), a system memory 1914 and a system bus 1916 that couples various system components including the system memory 1914 to the processing units 1912. The processing units 1912 may be any logic processing unit, such as one or more central processing units (CPUs) 1912a, digital signal processors (DSPs) 1912b, application-specific integrated circuits (ASICs), field programmable gate arrays (FPGAs), etc. The system bus 1916 can employ any known bus structures or architectures, including a memory bus with memory controller, a peripheral bus, and a local bus. The system memory 1914 includes read-only memory (ROM) 1918 and random access memory (RAM) 1922. A basic input/output system (BIOS) 1920, which can form part of the ROM 1918, contains basic routines that help transfer information between elements within the computer system 1900, such as during start-up.

The computer system 1900 may also include a plurality of interfaces such as network interface 1960 and interface 1950 supporting modem 1962 or any other wireless/wired interfaces.

The computer system 1900 may include a hard disk drive 1924 for reading from and writing to a hard disk 1926, an optical disk drive 1928 for reading from and writing to removable optical disks 1932, and/or a magnetic disk drive 1930 for reading from and writing to magnetic disks 1934. The optical disk 1932 can be a CD-ROM, while the magnetic disk 1934 can be a magnetic floppy disk or diskette. The hard disk drive 1924, optical disk drive 1928 and magnetic disk drive 1930 may communicate with the processing unit 1912 via the system bus 1916. The hard disk drive 1924, optical disk drive 1928 and magnetic disk drive 1930 may include interfaces or controllers (not shown) coupled between such drives and the system bus 1916, as is known by those skilled in the relevant art. The drives 1924, 1928 and 1930, and their associated computer-readable storage media 1926, 1932, 1934, may provide non-volatile and non-transitory storage of computer-readable instructions, data structures, program modules and other data for the computer system 1900.

Although the depicted computer system 1900 is illustrated employing the hard disk drive 1924, optical disk drive 1928 and magnetic disk drive 1930, those skilled in the relevant art will appreciate that other types of computer-readable storage media that can store data accessible by a computer may be employed, such as magnetic cassettes, flash memory, Bernoulli cartridges, RAMs, ROMs, smart cards, etc. For example, computer-readable storage media may include, but is not limited to, random access memory (RAM), read-only memory (ROM), electrically erasable programmable read-only memory (EEPROM), flash memory, compact disc ROM (CD-ROM), digital versatile disks (DVD) or other optical disk storage, magnetic cassettes, magnetic tape, magnetic disk storage or other magnetic storage devices, solid state memory or any other medium which can be used to store the desired information and which may be accessed by processing unit 1912a.

Program modules can be stored in the system memory 1914, such as an operating system 1936, one or more application programs 1938, other programs or modules 1940, and program data 1942. Application programs 1938 may include instructions that cause the processor(s) 1912 to generate, process, transmit, and/or receive scent messages 1612 and scent information. Other program modules 1940 may include instructions for handling security such as password or other access protection and communications encryption. The system memory 1914 may also include communications programs, for example, a Web server 1944 for permitting the computer system 1900 to be accessed and exchange data with Web browsers of the Internet, corporate intranets, extranets, or other networks and devices as described herein, as well as other server applications on server computing systems. The Web server 1944 in the depicted embodiment is markup language based, such as Hypertext Markup Language (HTML), Extensible Markup Language (XML) or Wireless Markup Language (WML), and operates with markup languages that use syntactically delimited characters added to the data of a document to represent the structure of the document. A number of Web clients or browsers are commercially available such as those from Mozilla, Google, and Microsoft.

While shown in FIG. 19 as being stored in the system memory 1914, the operating system 1936, application programs 1938, other programs/modules 1940, program data 1942 and server 1944 can be stored on the hard disk 1926 of the hard disk drive 1924, the optical disk 1932 of the optical disk drive 1928 and/or the magnetic disk 1934 of the magnetic disk drive 1930.

An operator can enter commands and information into the computer system 1900 through input devices such as keyboard 1946 and/or a pointing device such as a mouse 1948, and/or via a graphical user interface. Other input devices can include a microphone, joystick, game pad, tablet, scanner, etc. These and other input devices are connected to one or more of the processing units 1912 through an interface 1950 such as a serial port interface that couples to the system bus 1916, although other interfaces such as a parallel port, a game port or a wireless interface or a universal serial bus (USB) can be used. A monitor 1952 or other display device is coupled to the system bus 1916 via a video interface 1954, such as a video adapter. The computer system 1900 can include other devices, such as speakers, printers, etc.

The computer system 1900 can operate in a networked environment using logical connections to one or more remote computers and/or devices as described above with reference to FIG. 16. For example, the computer system 1900 can operate in a networked environment using logical connections to one or more mobile devices, landline telephones and other service providers or information servers. Communications may be via a wired and/or wireless network architecture, for instance wired and wireless enterprise-wide computer networks, intranets, extranets, telecommunications networks, cellular networks, paging networks, and other mobile networks.

It should be understood that the various techniques described herein may be implemented in connection with hardware, software and/or firmware or, where appropriate, with a combination of such. Thus, the methods and apparatuses of the disclosure, or certain aspects or portions thereof, may take the form of program code (i.e., instructions) embodied in tangible media, such as floppy diskettes, CD-ROMs, DVDs, hard drives, flash drives, or any other machine-readable or processor-readable storage medium wherein, when the program code is loaded into and executed by a machine, such as a processor of a computer or mobile device, the machine becomes an apparatus for practicing various embodiments. In the case of program code execution on programmable computers or mobile devices, such generally includes a processor, a storage medium readable by the processor (including volatile and non-volatile memory and/or storage elements), at least one input device, and at least one output device. One or more programs may implement or utilize the processes described in connection with the disclosure, e.g., through the use of an API, reusable controls, or the like. Such programs are preferably implemented in a high level procedural or object-oriented programming language to communicate with a computer system including the computer system 1900 depicted in FIG. 19. However, the program(s) can be implemented in assembly or machine language, if desired. In any case, the language may be a compiled or interpreted language, and combined with hardware implementations.

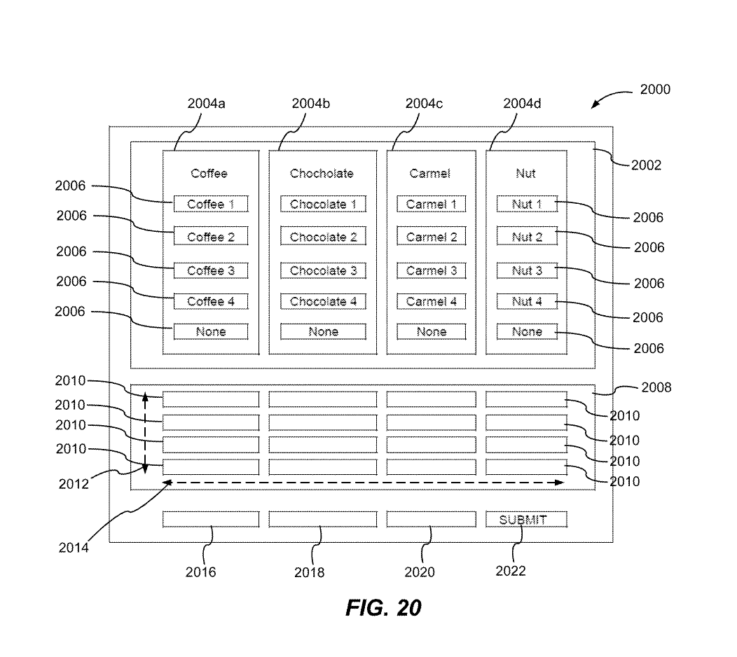

FIG. 20 is block diagram of a scent sequence specification user interface 2000 according to one illustrated embodiment. The scent sequence specification user interface 2000 may be provided by the server 1606. For example, the server 1606 may provide HTML formatted information corresponding to the scent sequence specification user interface 2000 to the second mobile device 1608. When the second mobile device 1608 receives the information, a processor of the device (e.g., processing unit 1812a) causes a display device (e.g., touch screen 1852) to display the scent sequence specification user interface 2000. When a user selects icons or inputs text (e.g., via the touch screen 1852), the processor of the second mobile device 1608 causes corresponding information to be transmitted to the server 1606 via the network 1610. As will be explained, a processor (e.g., processing unit 1912a) of the server 1606 executes predetermined instructions (e.g., that are stored in the application programs 1938) in response to receipt of information from the first mobile device 1604 and/or the second mobile device 1608.

The scent sequence specification user interface 2000 includes a scent palette 2002 that includes four categories of scents 2004a-2004d. The first category of scents 2004a includes four different coffee scents. The second category of scents 2004b includes four different chocolate scents. The third category of scents 2004c includes four different caramel scents. The fourth category of scents 2004d includes four different nut scents. The categories of scents 2004a-2004d include twenty selectable icons 2006, each of which indicates a particular scent (or absence of a scent). For example, one or more of the selectable icons 2006 may include text, symbols, and/or pictures that represent a particular scent.

The scent sequence specification user interface 2000 also includes a two-dimensional array of scent specification fields 2008. The array of scent specification fields 2008 includes sixteen text input boxes 2010. The text input boxes 2010 are arranged along a first axis 2012 and a second axis 2014, which is perpendicular to the first axis 2012. A distribution of the scent specifications fields (e.g., text input boxes 2010) along the first axis 2012 of the array of scent specification fields 2008 represents a position in a temporal sequence. For example, the scents specified via the text input boxes 2010 in the top row of the array of scent specification fields 2008 may be produced before the scents specified via the text input boxes 2010 in the bottom row of the array of scent specification fields 2008. A distribution of the scent specification fields (e.g., text input boxes 2010) along the second axis 2014 of the array of scent specification fields 2008 represents one or more scents to be dispensed at a respective position in the temporal sequence. For example, the scents specified via the text input boxes 2010 in a first row of the array of scent specification fields 2008 may be produced at a first time, the scents specified via the text input boxes 2010 in a second row of the array of scent specification fields 2008 may be produced at a second time, etc. That is, the scents specified via the text input boxes 2010 in each of the rows of the array of scent specification fields 2008 may represent a compound scent that is a mixture or combination of the scents specified via the input boxes 2010 in that row. Additionally, the scents specified via the text input boxes 2010 in the four illustrated rows of the array of scent specification fields 2008 may represent a sequence of four such compound scents.

When each of the selectable icons 2006 is selected, the server 1606 causes one of the text input boxes 2010 to display corresponding text. For example, if a user of the second mobile device 1608 selects the selectable icon 2006 labeled “Coffee 1,” the second mobile device 1608 sends the server 1606 information indicating that the selectable icon 2006 has been selected and, in response, the server 1606 causes the top text input box 2010 under the first category of scents 2004a to display “Coffee 1.” The scent sequence specification user interface 2000 also includes a text input box 2016, a text input box 2018, a text input box 2020, and a submit button 2022.

The server 1606 may initially enable the text input boxes 2010 in the first row (e.g., top row) of the two-dimensional array of scent specification fields 2008 to be populated, and then the scent specification fields 2008 in the second row, and so on. The scent sequence specification user interface 2000 may include a display area (not shown) in which instructions are displayed. The instructions may indicate that the scent sequence specification user interface 2000 can be used to generate up to four scent messages, each of which specifies a user-defined combination of up to four scents. The instructions may indicate that a theme or a name to be associated with the scent messages can be specified by entering text into the text input box 2016, that a sender to be associated with the scent messages can be specified by entering text into the text input box 2018, and that a recipient of the scent messages can be specified by entering text (e.g., an e-mail address) into the text input box 2020.

For example, a user of the second mobile device 1608 may initially select the selectable icons 2006 labeled “Coffee 1,” “Chocolate 1,” “Caramel 1,” and “Nut 1,” respectively, which causes the server 1606 to populate the text input boxes 2010 of the first row of the array of scent specification fields 2008 with text corresponding to the labels of the selectable icons 2006 that were selected. The user may then select the selectable icons 2006 labeled “Coffee 2,” “Chocolate 2,” “Caramel 2,” and “Nut 2,” respectively, which causes the server 1606 to populate the text input boxes 2010 of the second row of the array of scent specification fields 2008 with text corresponding to the labels of the selectable icons 2006 that were selected. The user may then select the selectable icons 2006 labeled “Coffee 3,” “Chocolate 3,” “Caramel 3,” and “None,” respectively, which causes the server 1606 to populate the text input boxes 2010 of the third row of the array of scent specification fields 2008 with text corresponding to the labels of the selectable icons 2006 that were selected. The user may then select the selectable icons 2006 labeled “Coffee 4,” “None,” “Caramel 1,” and “None,” respectively, which causes the server 1606 to populate the text input boxes 2010 of the fourth row of the array of scent specification fields 2008 with text corresponding to the labels of the selectable icons 2006 that were selected. The user may then enter a name or description to be associated with the selected scent combinations in the text input box 2016. The user also may enter her name in the text input box 2018, and an e-mail address of an intended recipient in the text input box 2020, and then select the submit button 2022.

In response, the server 1606 generates a scent specification file including a sequence of four scent messages 1612, each of which includes indicators of the scents specified in one of the rows of the array of scent specification fields 2008. The server 1606 also generates an e-mail message and attaches the scent specification file to the e-mail message and sends the e-mail message using the e-mail address input in the text input box 2020. The server 1606 also may include a default or user-specified duration (e.g., in seconds) for each scent message. For example, the scent generation file may include information corresponding to the information shown in Table 1 below.

TABLE 1

| Scent Specification Name = Alice's scent combinations | |
|---|---|
| From = Alice | |
| To = Bob@example.com | |
| Scent Message 1: | Scent Message 2: |
| Scent 1 = Coffee 1 | Scent 1 = Coffee 2 |
| Scent 2 = Chocolate 1 | Scent 2 = Chocolate 2 |
| Scent 3 = Caramel 1 | Scent 3 = Caramel 2 |
| Scent 4 = Nut 1 | Scent 4 = Nut 2 |
| Duration = 10 | Duration = 10 |
| Scent Message 3: | Scent Message 4: |
| Scent 1 = Coffee 3 | Scent 1 = Coffee 4 |
| Scent 2 = Chocolate 3 | Scent 2 = None |
| Scent 3 = Caramel 3 | Scent 3 = Caramel 1 |
| Scent 4 = None | Scent 4 = None |
| Duration = 10 | Duration = 10 |

When the e-mail message from the server 1606 is received at the first mobile device 1604, a user may open the message and select the attached file, which causes a processor of the first mobile device 1604 (e.g., processing unit 1812a) to execute an application for processing the scent generation file. The application may cause the processor to display a user interface on a touch screen (e.g., on the touch screen 1852). For example, the application may instruct the processor to cause a user interface to be displayed that displays a message indicating a name or theme of the scent messages (e.g., the text entered into the text input box 2016), from whom the message was sent (e.g., the text entered into the text input box 2018). Additionally, the displayed message may indicate the amount of time required for the scents specified in the scent generation file. For example, the processor may add the time durations specified in each scent message and cause the resulting sum to be displayed in the message. The user interface also may include selectable icons labeled “Continue,” and “Cancel,” for example. The displayed message may indicate that the user can select the “Continue” icon to continue processing the scent messages, and indicate that the user can select the “Cancel” icon at any time to cancel processing of the scent messages.

The scent sequence specification user interface 2000 may include categories of scents 2004a-2004d other than those described above. For example, the scents included in the scent sequence specification user interface 2000 may correspond to different flowers, and the scent sequence specification user interface 2000 may be used to create scent combinations and sequences that represent different bouquets of flowers.

Additionally, the scent sequence specification user interface 2000 may be used be to create paragraphs that convey ideas and/or emotions. Each paragraph may include a number of sentences, each of which is comprised of a number of words. Similarly, the scent sequence specification user interface 2000 can be used to generate symphonies of scents, wherein each symphony includes a paragraph.

For example, each of the scents may correspond to a scent that has been observed in a particular city, and the scent sequence specification user interface 2000 may be used to generate paragraphs that represent experiences in that city. A user of the second mobile device 1608 may use the scent sequence specification user interface 2000 to generate a paragraph and cause a corresponding scent specification file to be transmitted to a user of the first mobile device 1604 so that the scent delivery device 1062 can dispense a combination of scents in a particular sequence that is intended to remind the user of the first mobile device 1604 of a morning the two users had spent together in Paris. A first sentence included in the paragraph may include a number of words or particular scents observed while walking to a café (e.g., the scents of bread and other goods being baked). A second sentence may include a number of words or particular scents observed while dining in the café (e.g., the scents of coffee and croissants). A third sentence may include a number of words or particular scents observed while walking through a park on the way to the metro (e.g., the scents different trees). A fourth sentence may include a number of words or particular scents observed while riding the metro (e.g., the scents of subways and perfumes). A fifth sentence may include a number of words or particular scents observed while walking in the Jardin du Luxembourg (e.g., the scents of different flowers). Similarly, the user of the second mobile device 1608 may use the scent sequence specification user interface 2000 to generate another paragraph that is intended to remind the user of the first mobile device 1604 of another time the two users had spent together in Paris.

In one embodiment, the scent sequence specification user interface 2000 generates sentences according to predetermined grammatical rules. For example, the scent sequence specification user interface 2000 enables a user to select one or more of the selectable icons 2006 to specify: a scent or a scent combination that is the subject of a sentence, a scent or a scent combination that is the verb of the sentence, a scent or a scent combination that is a modifier of the subject of the sentence, and a scent or a scent combination that is a modifier of the verb of the sentence. In response to the user selections, the server 1606 generates information in which the scent corresponding to the subject is specified in one or more predetermined locations in the sequence (e.g., the first scent and the last scent of the sequence), the scent corresponding to the modifier of the subject is included immediately following or immediately the scent corresponding to the subject, the scent corresponding to the verb is repeated a predetermined number of times in the sequence (e.g., 3), and the scent corresponding to the modifier of the verb is included immediately before and/or immediately after each instance of the scent corresponding to the verb.

In one embodiment, the scent sequence specification user interface 2000 enables a user to specify information corresponding to the text portion 1614, the scent portion 1616, the audio portion 1618, and the video portion 1620 of a plurality of messages 1612. For example, the scent sequence specification user interface 2000 may be used to generate a message 1612 having a text portion 1614 that includes a text message, a scent portion 1616 that specifies a scent combination corresponding to the aroma of a particular coffee, an audio portion 1618 that specifies a recording of milk being frothed, and a video portion 1620 that specifies a picture of a cup of coffee. When the scent message 1612 is processed by the first mobile device 1604, a processing unit (e.g., processing unit 1812) of the first mobile device 1604 causes a display device to display the text corresponding to the text portion 1614, causes the scent dispensing device 1602 to dispense a scent corresponding to the scent portion 1616, causes speakers to emit sounds corresponding to the audio portion 1618, and causes the display device to display a picture corresponding to the video portion 1620. The audio portion 1618 and the video portion 1620 of the message 1612 may include references to attached files and/or links (e.g., URIs) to files that are stored by other devices (e.g., the server 1608).

Additionally, the scent sequence specification user interface 2000 can be used to generate a sequence of messages 1612 that correspond to a story, for example, a novel. An electronic or paper copy of such a novel may be accompanied by a group of scent cartridges (e.g., scent cartridges 900, 1200, or 1400) that include scents for creating the scent combinations specified in the scent portions 1616 of a group of messages 1612 corresponding to the novel. The novel and/or the scent cartridges may include a code or a link for accessing additional contents specified in the messages 1612. For example, a paper copy of the novel may include a bar code (e.g., a Quick Response Code) that, when scanned and decoded by the first mobile device 1604, causes the first mobile device 1604 to access a web page from which image files, audio files, and/or video files may be obtained. As described above, the first mobile device 1604 may process each message 1612 such that a scent specified in the scent portion 1616 is dispensed by the scent dispensing device 1602 while sounds specified in the audio portion 1618 are emitted by speakers and image(s) specified in the video portion 1620 are displayed by a display device.

In addition, a software application for processing the messages 1612 may cause information to be stored that can be used to resume the playback of the messages 1612. The software application may cause selectable icons to be displayed that enable a user to: specify one or more chapters of the novel to be played back, pause a playback at a current message 1612, and resume a playback at a message 1612 where the playback was previously paused. For example, the application may cause information identifying a sequence of messages 1612 corresponding to the novel to be stored in system memory (e.g., program data 1842) in association with an identifier of the next message 1612 that is to be processed, which may be incremented each time one of the messages 1612 is processed. When the user selects the icon for resuming the playback of a novel, the application may cause the first mobile device 1604 to obtain the stored identifier of the next message 1612 so that the playback is resumed at the proper message.

The software application also may cause information regarding an amount of usage of each scent medium included in each chamber of each scent cartridge to be stored. For example, the software application may cause a first variable and a second variable to be stored for each scent medium, wherein the first variable indicates a number of seconds of actual use time and the second variable indicates a total number of seconds of expected use time. The software application may cause the first variable to be incremented each time the scent medium is used, and cause the second variable to be initialized to a particular value when a scent cartridge containing the scent medium is loaded into a scent dispensing device (e.g., scent dispensing device 1602). Additionally, the software application may cause a message to be displayed when the actual use time indicated by the first variable exceeds a predetermined threshold (e.g., 90% of the total expected use time indicated by the second variable).

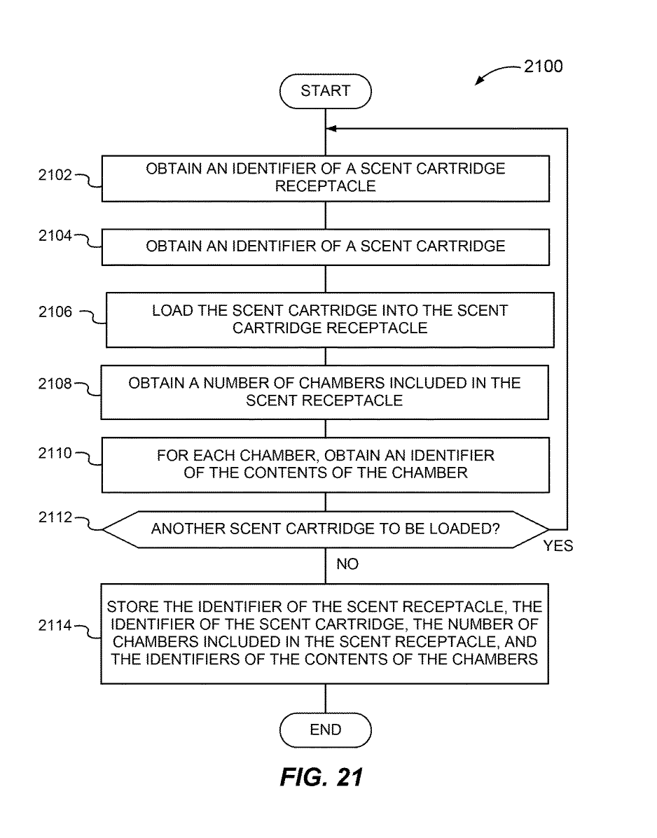

FIG. 21 is a flowchart of a method 2100 of operating the scent dispensing system 1600 shown in FIG. 16, according to one illustrated embodiment. One or more acts included in the method 2100 may be performed by the first mobile device 1604, for example, after a processing unit (e.g., processing unit 1812) of the first mobile device 1604 loads a software application including computer-executable instructions into RAM (e.g., RAM 1822) and executes the instructions. The application may instruct the processing unit to cause a touch screen (e.g., touch screen 1852) to display a series of prompts, receive information input in response to the prompts, and store the received information in program data in system memory (e.g., program data 1842).

At 2102, the processing unit of the first mobile device 1604 causes the touch screen to display a prompt requesting the user to enter an identifier of a scent cartridge receptacle in which a scent cartridge is to be loaded. For example, the identifier of the scent cartridge receptacle may be embossed, printed, provided on a label, or is otherwise visible to the user on an upper surface of one of the axels 1012c of one of the scent cartridge receptacles 1012b of the scent cartridge holder 1012 shown in FIG. 11A. The user may enter the identifier of the scent cartridge receptacle via the touch screen. In response, the processor obtains the identifier of the scent cartridge receptacle and stores it in RAM.

At 2104, the processing unit of the first mobile device 1604 causes the touch screen to display a prompt requesting the user to enter an identifier of the scent cartridge that is to be loaded into the scent cartridge receptacle. For example, the identifier of the scent cartridge may be embossed, printed, provided on a label, or is otherwise visible to the user on an exterior portion of the scent cartridge. The user may enter the identifier of the scent cartridge via the touch screen. In response, the processor obtains the identifier of the scent cartridge and stores it in RAM.

In one embodiment, at 2104, the processing unit of the first mobile device 1604 causes the touch screen to display a prompt requesting the user to take a picture using a camera (e.g., camera unit 1856) of a computer-readable symbol (e.g., barcode) included on the housing 1202 of the scent cartridge 1200 shown in FIG. 12A, or that is included in the contents indicator 1414 formed on the outer surface of the main portion 1410 of the hermetically sealed membrane 1408 shown in FIG. 14A, for example. The processing unit obtains the identifier of the scent cartridge by processing image data corresponding to an image of the computer-readable symbol using conventional techniques.

At 2106, the user loads the scent cartridge into the scent cartridge identified at 2104 into the scent cartridge receptacle of the scent dispensing device 1602 identified at 2102. For example, the user loads the scent cartridge 1200 into one of the scent cartridge receptacles 1012b of the scent cartridge holder 1012 show in FIG. 11A.

At 2108, the processing unit causes the touch screen to display a prompt requesting the user to enter a number of chambers included in the scent cartridge that was loaded into the scent cartridge receptacle of the scent dispensing device 1602 at 2102. In response, the processor obtains the number of chambers and stores the number in RAM. In one embodiment, the processor obtains the number of chambers from image data obtained at 2104.

At 2110, the processing unit causes the touch screen to display a series of prompts requesting the user to enter an identifier of the contents of each of the chambers included in the scent cartridge that was loaded into the scent cartridge receptacle at 2106. In response, the processor obtains the identifiers of the contents the chambers and stores them in RAM. In one embodiment, the processor obtains the identifiers of the contents of the chambers from image data obtained at 2104. In another embodiment, the processing unit 1812 obtains the identifiers of the contents each of the chambers from system memory. For example, the processing unit obtains the identifiers of the contents of each of the chambers from a table stored in program data, which is associated with the identifier of the scent cartridge obtained at 2104.

At 2112, the processing unit determines whether another scent cartridge is to be loaded into the scent dispensing device 1602. For example, the processing unit causes the touch screen to display a prompt requesting the user to select a selectable icon to indicate whether another scent cartridge is to be loaded. If the user indicates via the touch screen that another scent cartridge is to be loaded, the method returns to 2102. If the user indicates via the touch screen that no other scent cartridge is to be loaded, the method proceeds to 2114.

At 2114, the processing unit causes one or more of the following to be stored in system memory: the identifier of the scent cartridge receptacle obtained at 2102, the identifier of the scent cartridge obtained at 2104, the number of chambers included in the scent cartridge receptacle obtained at 2108, and the identifiers of the contents of the chambers obtained at 2110. For example, the processing unit causes information corresponding to Table 2 or Table 3 below to be stored in program data at 2110.

TABLE 2

| Scent Cartridge Receptacle Identifier = A | |
|---|---|
| Scent Cartridge Identifier = CHO001 | |
| Chamber Number | Content Identifier |
| 1 (default) | Empty |
| 2 | Chocolate 1 |
| 3 | Chocolate 2 |
| 4 | Chocolate 3 |
| 5 | Chocolate 4 |

TABLE 3

| Scent Cartridge Receptacle Identifier = B | |
|---|---|
| Scent Cartridge Identifier = COF001 | |
| Chamber Number | Content Identifier |
| 1 (default) | Empty |
| 2 | Coffee 1 |
| 3 | Coffee 2 |
| 4 | Coffee 3 |
| 5 | Coffee 4 |

After the information is stored at 2114, the touch screen of the first mobile device 1604 may display a selectable icon that, when selected, causes the processing unit to display a user interface that enables a user to specify scents to be dispensed by the scent dispensing device 1602. For example, the user interface may include a plurality of selectable icons, wherein each selectable icon includes one of the identifiers of the contents of the chambers obtained at 2110. In response to each of the icons being selected, the processing unit generates a message including a scent specification that identifies a scent corresponding to the selected icon and transmits the message to the scent dispensing device 1602. After the scent dispensing device 1602 receives each scent specification, the scent dispensing device 1602 causes a corresponding scent to be dispensed, as will be explained below.

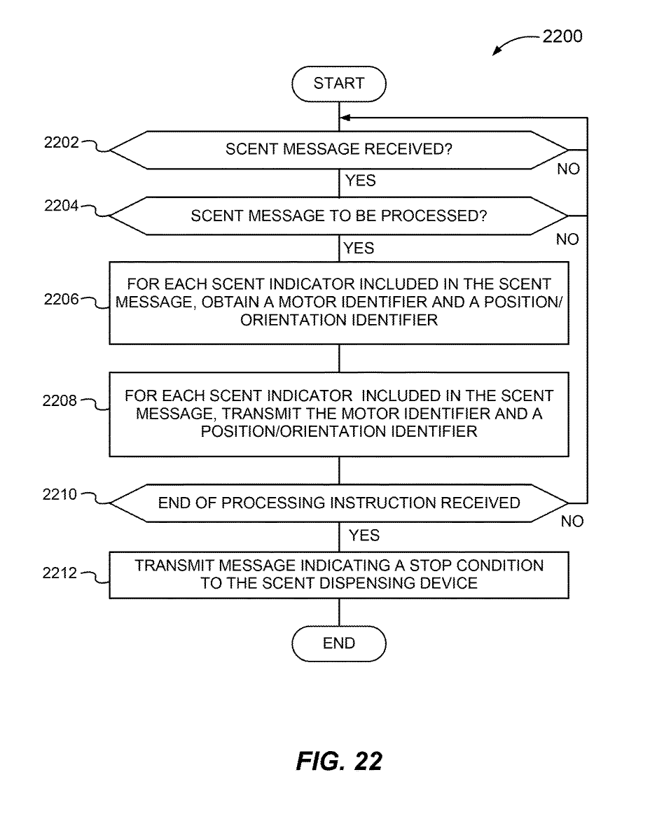

FIG. 22 is a flowchart of a method 2200 of operating the scent dispensing system 1600, according to one illustrated embodiment. One or more acts included in the method 2200 may be performed by the first mobile device 1604, for example, after a processing unit (e.g., processing unit 1812) of the first mobile device 1604 loads a software application including computer-executable instructions into RAM (e.g., RAM 1822) and executes the instructions. The application may instruct the processing unit to cause a touch screen (e.g., touch screen 1852) to display a series of prompts, receive information input in response to the prompts, store the received information in RAM (e.g., RAM 1822) of system memory (e.g., system memory 1814), process and/or transform the received information, and transmit information corresponding to the received information via a network interface (e.g., network interface 1860).

At 2202, the processing unit determines whether a scent message has been received. For example, the processing unit inspects a message buffer in the system memory for the presence of a scent message. If the processing unit determines that a scent message has not been received, the method returns to 2202. If the processing unit determines that a scent message has been received, the method proceeds to 2204.

At 2204, the processing unit determines whether the scent message is to be processed. For example, the processing unit 1812 causes the touch screen to display a prompt requesting the user to select a selectable icon indicating whether the scent message is to be processed. If the processing unit determines that the scent message is not to be processed, the method returns to 2202. If the processing unit determines that the scent message is to be processed, the method proceeds to 2206.

At 2206, the processing unit parses the scent message and identifies each scent indicator included in the scent message. Each scent indicator may indicate, for example, a particular scent (e.g., Chocolate 1) or a lack of particular scent (e.g., an empty chamber or a chamber including a particular type of air filter). For example, the processing unit obtains a first scent identifier of “Coffee 3” and a second scent identifier of “Chocolate 2” from the scent message. The processing unit then obtains a scent cartridge receptacle identifier and a chamber identifier that correspond to the first scent identifier. For example, the processing unit accesses information corresponding to Table 3 above and determines that the scent “Coffee 3” is included in chamber number 4 of the scent cartridge loaded in the scent cartridge receptacle “B,” and also information corresponding to Table 2 above and determines that the scent “Chocolate 2” is included in chamber number 3 of the scent cartridge loaded in the scent cartridge receptacle “A.” The processing unit then obtains the corresponding motor identifiers and position identifiers from information corresponding to Tables 4 and 5 below, for example. That is, the processing unit obtains, from information corresponding to Table 4, a motor identifier of “00,” which corresponds to the scent cartridge receptacle identifier “A” and a motor identifier of “01,” which corresponds to the scent cartridge receptacle identifier “B.” The processing unit also obtains, from information corresponding to Table 5, a position/orientation identifier of “010,” which corresponds to the chamber number 3, and a position/orientation identifier of “011,” which corresponds to the chamber number 4.

TABLE 4

| Scent cartridge | |
|---|---|
| receptacle | Motor |
| Identifier | Identifier |
| A | 00 |
| B | 01 |
| C | 10 |
| D | 11 |

TABLE 5

| Chamber | Orientation/ |
|---|---|
| Number | Position Identifier |
| 1 | 000 |
| 2 | 001 |
| 3 | 010 |
| 4 | 011 |
| 5 | 100 |

At 2208, the processing unit transmits the motor identifier and the position/orientation identifier obtained at 2206 for each scent identifier included in the scent message to the scent dispensing device 1602. The processing unit may cause a network interface for Bluetooth communications (e.g., network interface 1860) to transmit a first message for the first scent identifier and a second message for the second scent identifier to the scent dispensing device 1602. For example, for the first scent identifier of “Coffee 3,” the first message includes a motor identifier of “01” and a position/orientation identifier of “011” and, for the second scent identifier of “Chocolate 2,” the second message includes a motor identifier of “00” and a position/orientation identifier of “010.” The first and the second messages may include other information, for example, network addresses of the scent dispensing device 1602 and the first mobile device 1604. The first and the second messages may also include other information, such as control information indicating whether each message is the first or last message of a group of messages that create a particular scent combination or sequence of scent combinations.

At 2210, the processing unit determines whether an end of processing instruction has been received. For example, the processing unit 1812 causes the touch screen to display a selectable icon that, when selected, causes the processing unit to receive the end of processing instruction. If the processing unit determines that the end of processing instruction has not been received, the method returns to 2202. If the processing unit determines that the end of processing instruction has been received, the method proceeds to 2212.

At 2212, the processing unit 1812 causes the network interface to transmit a predefined message that indicates a stop condition to the scent dispensing device 1602. The method 2200 ends after the message is transmitted at 2212.

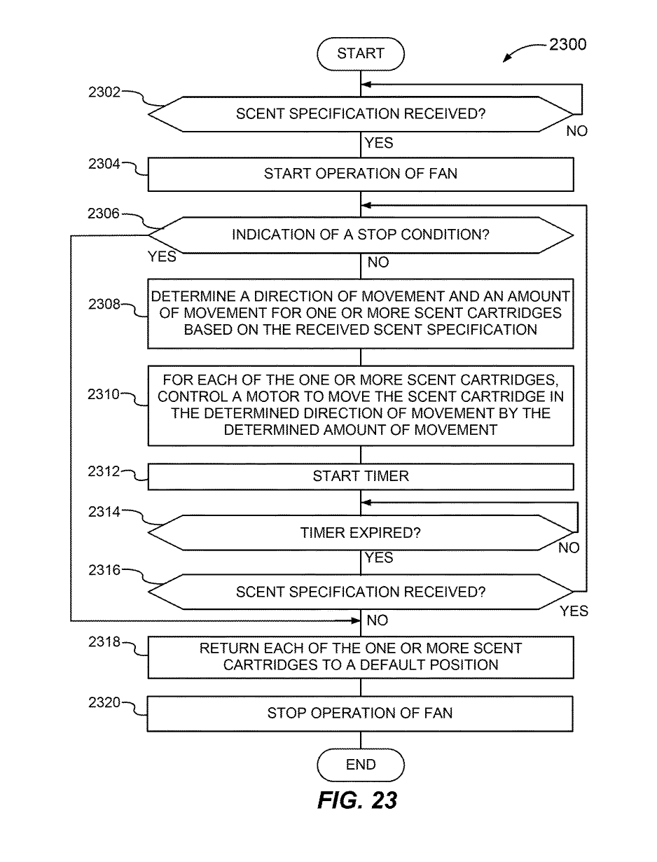

FIG. 23 is a flowchart of a method 2100 of operating the scent dispensing system 1600 shown in FIG. 16, according to one illustrated embodiment. One or more acts included in the method 2300 may be performed by the scent dispensing device 1602, for example, after a user operates a switch (e.g., slide potentiometer 1706) that causes power to be supplied to the scent dispensing device 1602. In response, a microcontroller (e.g., microcontroller 1702) causes processor-executable instructions stored in an internal memory to be loaded into an internal RAM and begins executing the instructions.

At 2302, the microcontroller determines whether a scent specification has been received. For example, the microcontroller may inspect an internal Universal Asynchronous Receiver/Transmitter (UART) to determine whether a message from the first mobile device 1604 has been received via a Bluetooth communications module (e.g., Bluetooth communications module 1710). The received scent specification may be included in, or correspond to, information included in a scent specification file generated using the scent sequence specification user interface 2000 shown in FIG. 20, for example. If the microcontroller determines that the scent specification has not been received, the method returns to 2302. If the microcontroller determines that the scent specification has been received, the method proceeds to 2304.

At 2304, the microcontroller starts operation of a fan (e.g., fan 1708) by causing an appropriate control signal to be provided to the fan. The particular control signal provided to the fan may depend on the position of a switch (e.g., slide potentiometer 1706). For example, if the switch is at a first position, the microcontroller provides a first control signal to the fan and, if the switch is at a second position, the microcontroller provides a second control signal to the fan, wherein the first control signal causes the fan to rotate faster than the second control signal.

At 2306, the microcontroller determines whether an indication of a stop condition is present. For example, the microcontroller monitors a particular input/output line that is connected to a circuit that provides an indication when the charge remaining in a battery that supplies power to the scent dispensing device 1602 falls below a predetermined threshold. The predetermined threshold is selected such that the charge remaining in the battery is sufficient to move each of the motors and thus each of the scent cartridges of the scent dispensing device 1602 to a default position or orientation. At 2306, the microcontroller also may determine whether a predetermined message indicating a stop condition has been received from the first mobile device 1604. If the microcontroller determines that the indication of the stop condition is present, the method proceeds to 2318. If the microcontroller determines that the indication of the stop condition is not present, the method proceeds to 2308.

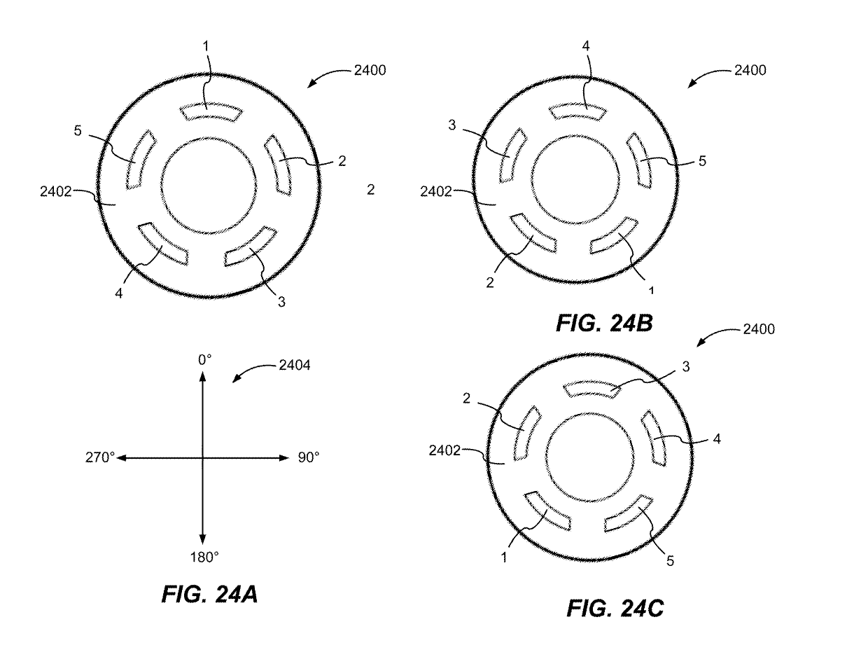

At 2308, the microcontroller determines a direction of movement and an amount of movement of one or more cartridges and/or motors. The acts performed at 2308 will be described with reference to FIGS. 24A-24C. FIG. 24A shows a top plan view of a scent cartridge 2400, according to one illustrated embodiment. An end portion 2402 of the cartridge 2400 includes five apertures that function as outlet ports for five chambers numbered 1-5. The cartridge 2400 shown in FIG. 24A is in a default orientation or position, in which the aperture corresponding to chamber 1 is aligned with an inlet port of a scent mixing chamber (not shown) of the scent dispensing device 1602. A reference coordinate system 2404 also is shown in FIG. 24A.

For example, the aperture corresponding to chamber 1 may be aligned with an aperture 1014c formed in the bottom portion 1014b of the scent mixing chamber 1014 shown in FIG. 10E. More particularly, the cartridge 2400 may be positioned below the scent mixing chamber 1014 such that a lower surface of the bottom portion 1014b of the scent mixing chamber 1014 is parallel to an upper surface of the end portion 2402 of the cartridge 2400, and the cartridge 2400 is rotated, positioned, or otherwise oriented such that the aperture corresponding to chamber 1 is vertically aligned with the aperture 1014c. For example, the cross-sectional areas of the aperture corresponding to chamber 1 the aperture 1014c are sized and dimensioned the same and, if the cross-sectional area of the aperture 1014c is projected downwardly, the projected area would pass through the aperture corresponding to chamber 1 and would not pass through other areas of the end portion 2402.

Table 6 below shows an example of information that may be stored in internal memory of the microcontroller. In one embodiment, the apertures in the end portion 2402 of the cartridge 2400 have the same size and dimensions, and are uniformly spaced on the end portion 2402 of the cartridge 2400. The orientation of the center of each chamber can be defined by the orientation of a line that starts at the center of the end portion 2402 of the cartridge 2400 and passes through the center of the aperture corresponding to the chamber. The microcontroller can cause the shaft of stepper motors (e.g., stepper motors 1704) to move one step at a time, wherein 200 steps cause one full rotation of the shaft. Table 6 also includes the number of steps that are required to rotate the cartridge 2400 counterclockwise from the default position shown in FIG. 24A such that a particular chamber is aligned with one of the apertures 1014c formed in the bottom portion 1014b of the scent mixing chamber 1014, for example.

TABLE 6

| Chamber | Orientation | Orientation of Center | Relative |
|---|---|---|---|
| Number | Identifier | of Chamber | Steps |
| 1 | 000 | 0° | 0 |
| 2 | 001 | 72° | 40 |
| 3 | 010 | 144° | 80 |
| 4 | 011 | 216° | 120 |
| 5 | 100 | 288° | 160 |

Table 7 shows a table similar to Table 6 that is for clockwise rotation. Information corresponding to Table 7 may be stored in internal memory of the microcontroller.

TABLE 7

| Chamber | Orientation | Orientation of Center | Relative |
|---|---|---|---|
| Number | Identifier | of Chamber | Steps |
| 1 | 000 | 0° | 0 |
| 2 | 001 | 288° | 160 |
| 3 | 010 | 216° | 120 |
| 4 | 011 | 144° | 80 |
| 5 | 100 | 72° | 40 |

The processing performed at 2308 will be explained with reference to a series of calculations performed by the scent delivery device 1602 in response to receiving a scent specification corresponding to a scent specification file, for example, generated using the scent sequence specification user interface 2000 shown in FIG. 20 and described above. The scents specified in a sequence of scent specifications require the scent dispensing device 1602 to determine corresponding orientations of the scent cartridge 2400. For example, the scent cartridge 2400 is initially a default or first orientation or position shown in FIG. 24A, the scent dispensing device 1602 determines a second orientation of the scent cartridge 2400 corresponding to FIG. 24B, and then a third orientation of the scent cartridge 2400 corresponding to FIG. 24C.

As noted above, the scent cartridge 2400 initially is in the default orientation shown in FIG. 24A, and the scent dispensing device 1602 determines the second orientation corresponding to FIG. 24B. For example, the scent specification includes information indicating that the aperture corresponding to chamber number 4 of the scent cartridge 2400 is to be aligned with aperture 1014c formed in the bottom portion 1014b of the scent mixing chamber 1014. The microcontroller first determines the amount of rotation required if the scent cartridge 2400 is rotated in a clockwise direction and the amount of rotation required if the scent cartridge 2400 is rotated in a counterclockwise direction. That is, the microcontroller determines that the scent cartridge 2400 shown in FIG. 24A must be rotated by 144° (or 80 steps) in the clockwise direction, or alternatively must be rotated by 216° (or 120 steps) in the counterclockwise direction, for the scent cartridge 2400 to become oriented as shown in FIG. 24B. Because less rotation of the scent cartridge 2400 is required in the clockwise direction (e.g., fewer steps), the microcontroller determines at 2308 that the direction of movement is the clockwise direction, and that the amount of movement is 144° (or 80 steps).

At 2310, the microcontroller controls the motor coupled to the scent cartridge 2400 to move in the direction of movement determined at 2308, by the amount of movement determined at 2308. For example, the microcontroller 1702 provides control signals to the stepper motor 1704a that cause the shaft of the stepper motor 1704a to move 80 steps in the clockwise direction to orient the scent cartridge 2400 as shown in FIG. 24B.

At 2312, the microcontroller starts an internal timer. In one embodiment, the microcontroller initializes the timer started at 2312 using a default timer value (e.g., 5000 milliseconds). In another embodiment, the microcontroller initializes the timer started at 2312 using a timer value (e.g., 10 seconds) that is included in the scent specification received at 2302.

At 2314, the microcontroller determines whether the timer has expired. For example, the microcontroller monitors the timer for a predetermined voltage level that indicates that the amount of time used to initialize the timer has elapsed after the timer was set at 2312. If the microcontroller 1702 determines that the timer has not expired, the method returns to 2314. If the microcontroller 1702 determines that the timer has expired, the method proceeds to 2316.

At 2316, the microcontroller determines whether an additional scent specification has been received. For example, the microcontroller performs the same acts as described above in connection with 2302. If the microcontroller determines that the additional scent specification has been received, the method returns to 2306.

For example, at 2306, the microcontroller determines that the indication of the stop condition has not been received and proceeds to 2308. At 2308, the microcontroller determines the third orientation corresponding to FIG. 24C. For example, the scent specification includes information indicating that the aperture corresponding to chamber number 3 of the scent cartridge 2400 is to be aligned with aperture 1014c formed in the bottom portion 1014b of the scent mixing chamber 1014. The microcontroller determines the amount of rotation required if the scent cartridge 2400 is rotated in the clockwise direction and the amount of rotation required if the scent cartridge 2400 is rotated in the counterclockwise direction. That is, the microcontroller determines that the scent cartridge 2400 shown in FIG. 24B must be rotated by 72° (or 40 steps) in the clockwise direction, or alternatively must be rotated by 288° (or 160 steps) in the counterclockwise direction, for the scent cartridge 2400 to become oriented as shown in FIG. 24C. Because less rotation of the scent cartridge 2400 is required in the clockwise direction, the microcontroller determines at 2308 that the direction of movement is the clockwise direction, and that the amount of movement is 72° (or 40 steps). The microcontroller then performs the acts described above at 2310, 2312, and 2314.

At 2316, if the microcontroller determines that the additional scent specification has not been received, the method proceeds to 2318. At 2318, the microcontroller returns each cartridge that was moved based on the controlling performed at 2310 to its default orientation or position. For example, if the cartridge 2400 is currently oriented/positioned as shown in FIG. 24C, the microcontroller causes the cartridge 2400 to be oriented/positioned as shown in FIG. 24A. That is, the microcontroller performs processing similar to that performed at 2308 and 2310 to cause the cartridge 2400 to be oriented/positioned as shown in FIG. 24A.

At 2318, the microcontroller causes the fan to stop operating. For example, the microcontroller 1702 provides a control signal to the fan 1708 that causes the fan 1708 to turn off.

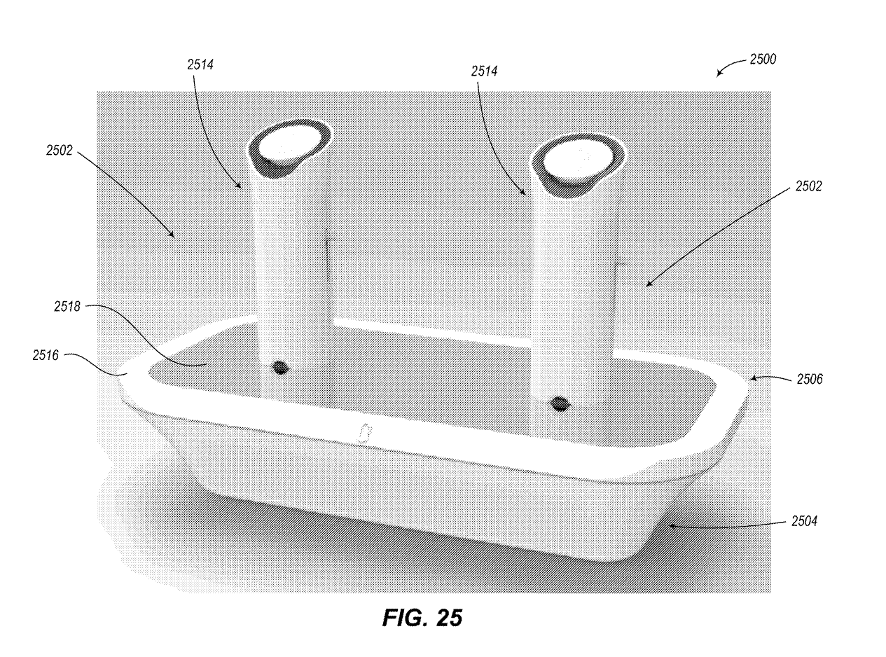

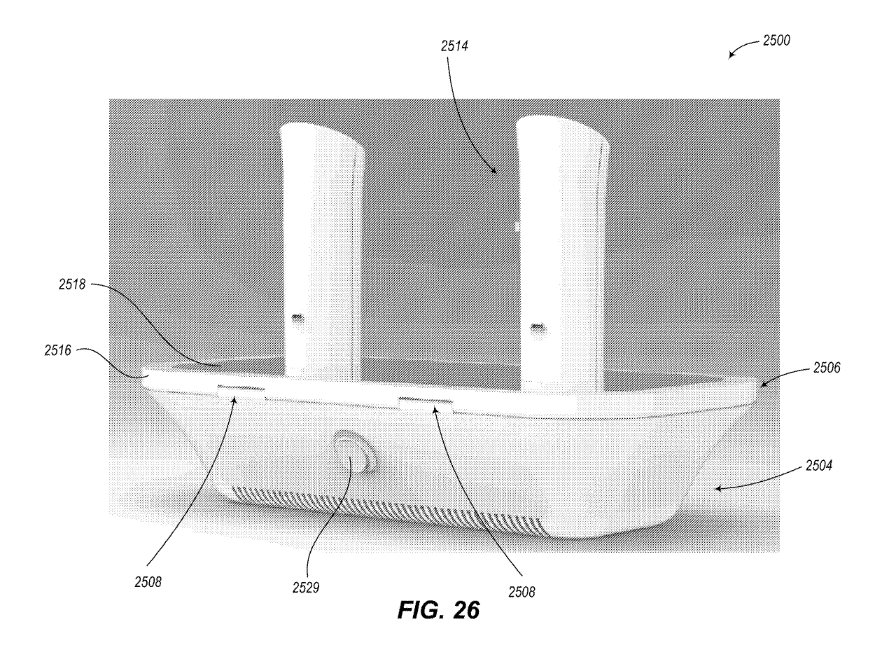

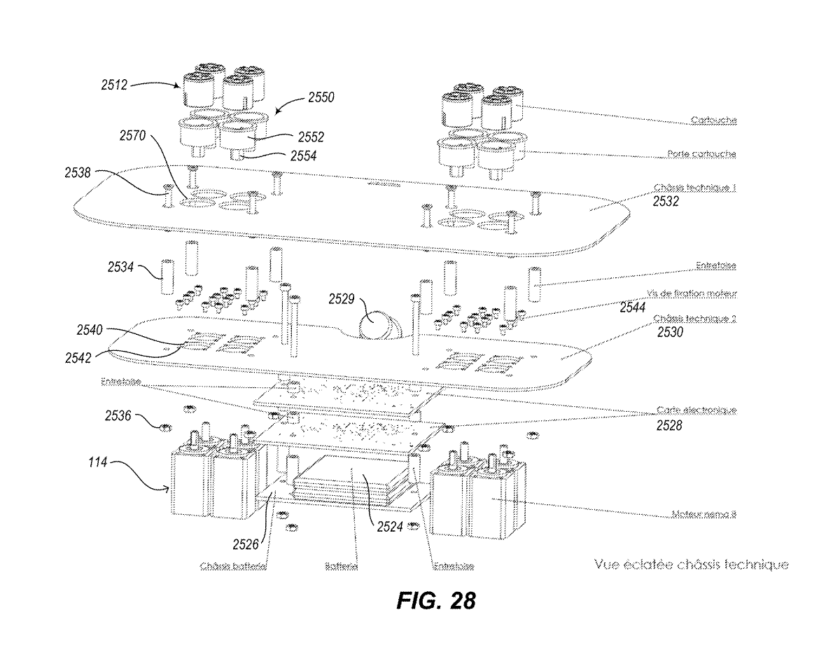

FIGS. 25-28 show a scent dispensing device 2500 according to one illustrated embodiment. The scent dispensing device 2500 is similar in many respects to the scent dispensing device 100 of FIG. 1A, so much of the discussion above regarding the scent dispensing device 100 is also applicable to the scent dispensing device 2500. Generally, the scent dispensing device 2500 includes two scent dispensing units 2502, a base 2504, and a lid 2506 rotatably coupled to the base via hinges 2508. Each scent dispensing unit 2502 includes four motors 114 (FIG. 28), four consumable scent cartridges 2512, a scent emitter 2514, and a number of electronic components that are described below. The motor 114 is discussed above with reference to FIGS. 7A and 7B.

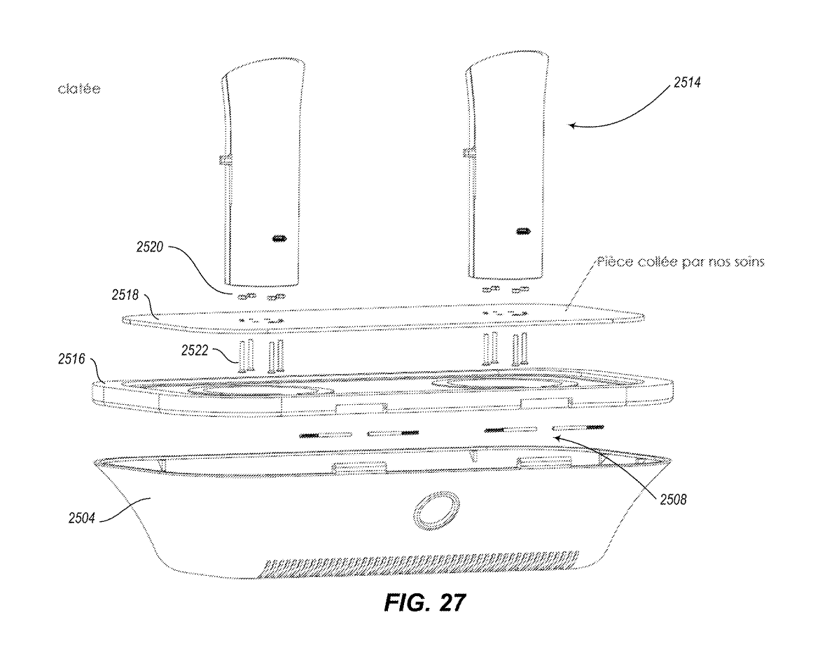

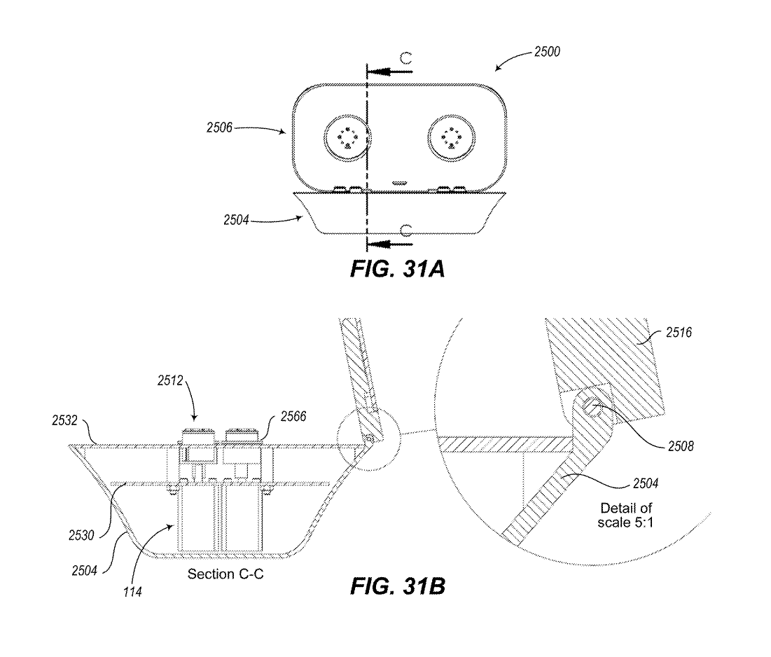

As shown in FIG. 27, the lid 2506 includes a supporting frame 2516 and a top shelf 2518 positioned thereon. Two scent emitters 2514 are attached to the top shelf using a plurality of nuts 2520 and bolts 2522. The top shelf 2518 may be fixedly or removably coupled to the supporting frame 2516 of the lid 2506. As shown best in FIGS. 31A-B and 32A-B, the hinges 2508 permit the lid 2506 to rotate relative to the base 2504 into an open position.

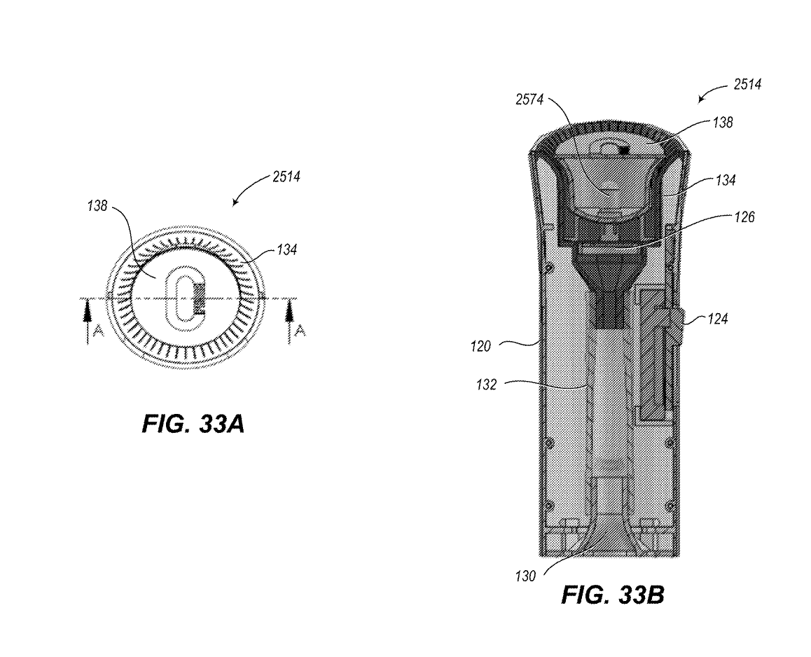

As shown in FIG. 28, the scent dispensing device 2500 also includes one or more batteries 2524 disposed on a battery support frame 2526 and one or more printed circuit boards 2528. The printed circuit boards 2528 include various components and circuits for communicating and processing scent information, and for controlling operation of the motors 114 and a fan 126 (FIG. 33B) based on such scent information. The scent dispensing device 2500 may also include a button 2529 that allows the user to turn the scent dispensing device 2500 ON and OFF by controlling power to one or more components on the printed circuit boards 2528. A lower shelf or frame 2530 of the base rests upon the motors 114. The lower frame 2530 is coupled to an upper frame 2532 of the base by spacers 2534, nuts 2536, and bolts 2538.

As discussed above, each of the motors 114 includes a motor housing 114a and a shaft 114b that extends upwardly from the motor housing 114a (FIGS. 7A and 7B). The shaft 114b includes a flat portion 114c and a truncated cylindrical portion 114d. Each of the motors 114 includes a top portion 114e having a plurality of apertures 114f.

As shown in FIG. 28, a portion of the lower shelf 2530 includes four large circular apertures 2540 and sixteen small circular apertures 2542. Eight motors 114 are attached to the lower shelf 2530 using a plurality of bolts 2544. A process for attaching one of the motors 114 to the lower shelf 2530 will now be described with reference to FIG. 28. One of the motors 114 is placed below the lower shelf 2530 such that the circular apertures 114f of the motor 114 are aligned with a group of four small circular apertures 2542 surrounding one of the large circular apertures 2540 of the lower shelf 2530. The motor 114 is advanced toward the lower shelf 2530 and the shaft 114b is passed through the large circular aperture 2540 until the top portion 114e of the motor 114 abuts the bottom surface of the lower shelf 2530. Four fasteners (e.g., bolts 2544) are inserted through respective small circular apertures 2542 of the lower shelf 2530 and are advanced into the circular apertures 114f of the motor 114. The bolts 2544 are rotated to engage threads (not shown) within the motor housing 114a thereby attaching the motor 114 to the lower shelf 2530. The above-described process is repeated for the remaining seven motors 114.

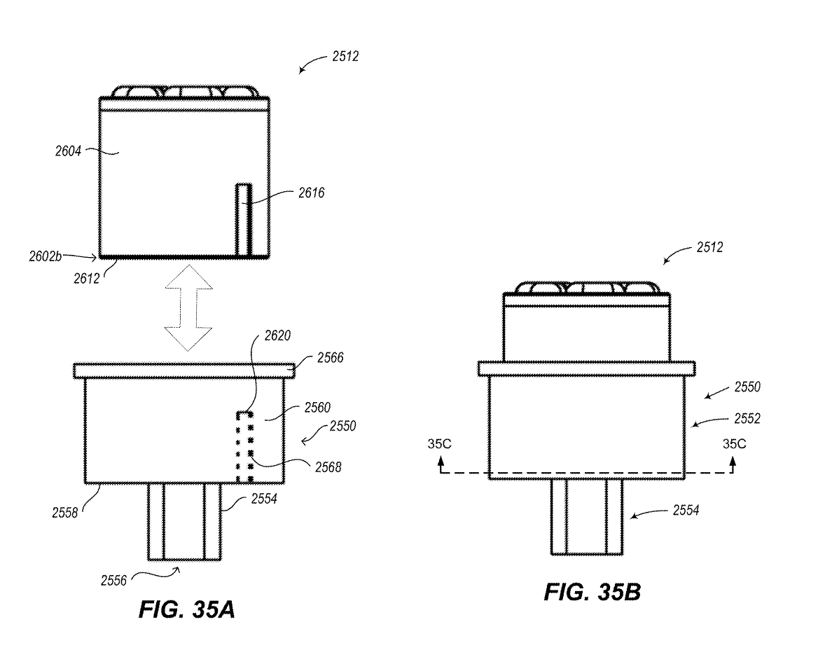

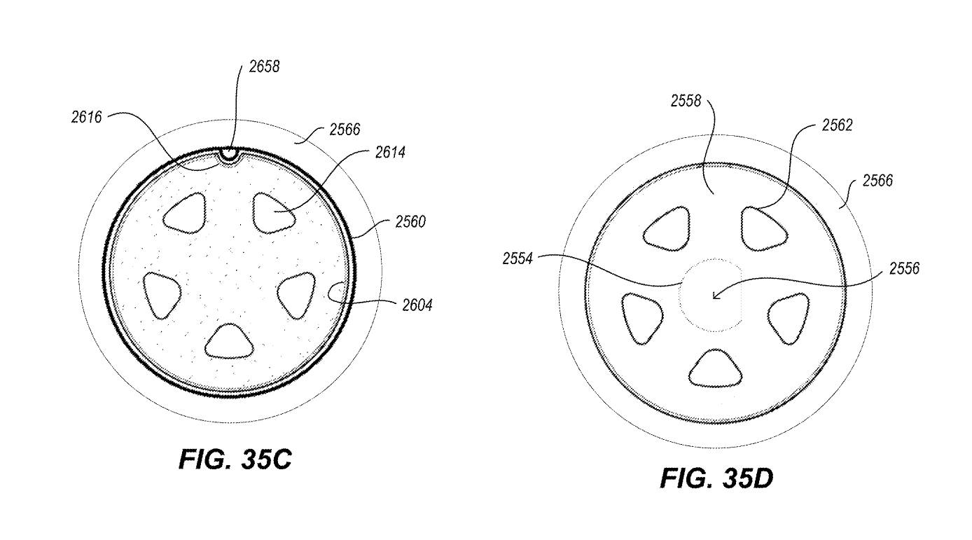

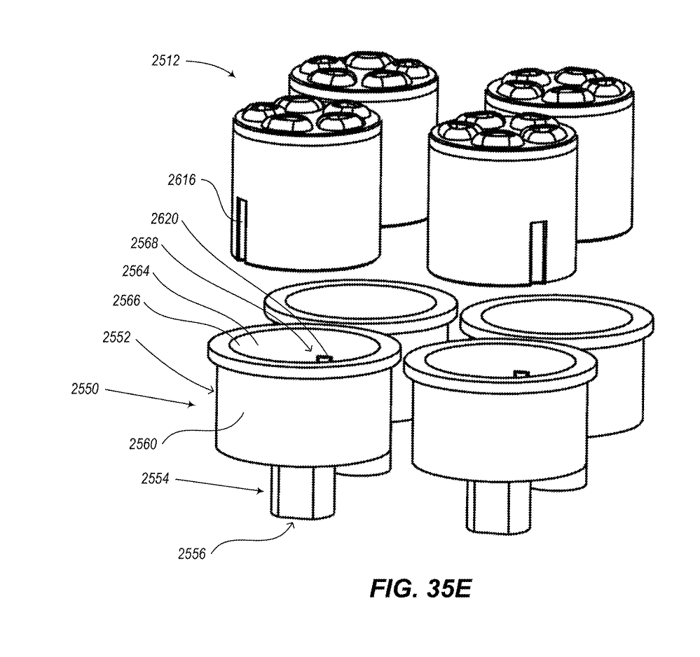

The scent dispensing device 2500 includes eight scent cartridge receiving portions 2550, also referred to herein as coupling sleeves. The scent cartridge receiving portions 2550 may best be viewed in FIGS. 35A-E. Each scent cartridge receiving portion 2550 comprises a body portion 2552 and a motor shaft coupling portion 2554 disposed below the body portion. As shown in FIG. 35D, the motor shaft coupling portion 2554 includes downward facing “D” shaped aperture 2556. The aperture 2556 of the motor shaft coupling portion 2554 is sized and dimensioned such that the shaft 114b of the motors 114 can be inserted into the aperture. When the shaft 114b of one of the motors 114 is inserted into the aperture 2556 of the motor shaft coupling portion 2554, the “D” shaped shaft 114b of the motor engages the “D” shaped aperture 2556 of the motor shaft coupling portion, which ensures that rotation of the shaft 114d causes rotation of the scent cartridge receiving portion 2550 about a vertical axis.

As shown best in FIGS. 35A-D, the body portion 2552 of the scent cartridge receiving portion 2550 comprises a base portion 2558 and a cylindrical wall 2560 extending upwardly therefrom. The base portion 2558 includes five wedge-shaped apertures 2562 (FIG. 35D). The cylindrical wall 2560 has an interior surface 2564 (FIG. 35E) sized and dimensioned to snuggly receive a lower portion of the scent cartridge 2512, as discussed below. A flange or lip 2566 extends around an uppermost portion of the cylindrical wall 2560. The interior surface 2564 of the cylindrical wall 2560 of the body portion 2552 comprises an inward facing projection or key 2568 (FIGS. 35C and 35E) extending inward from the interior surface 2564. The key 2568 is shown in dashed lines in FIG. 35A. As discussed further below, the key 2568 slidably engages a corresponding keyway of the scent cartridge 2512 to align the scent cartridge and restrict rotational movement thereof with respect to the scent cartridge receiving portion 2550.

A process for attaching one of the scent cartridge receiving portion 2550 to the scent dispensing device 2500 will now be described with reference to FIGS. 28 and 31B. When the upper shelf 2532 is attached to the lower shelf 2530 using the bolts 2538, nuts 2536 and spacers 2534, one of the scent cartridge receiving portions 2550 is advanced toward one of the shafts 114b of one of the motors 114. The motor shaft coupling portion 2554 and most of the body portion 2552 of the scent cartridge receiving portion 2550 passes through a large circular aperture 2570 in the top shelf 2532 until the “D” shaped shaft 114b of the motor 114 engages the “D” shaped aperture 2556 of the motor shaft coupling portion 2554 and the bottom surface of the flange 2566 abuts the top surface of the upper shelf 2532. The above-described process is repeated for the remaining seven scent cartridge receiving portions 2550.

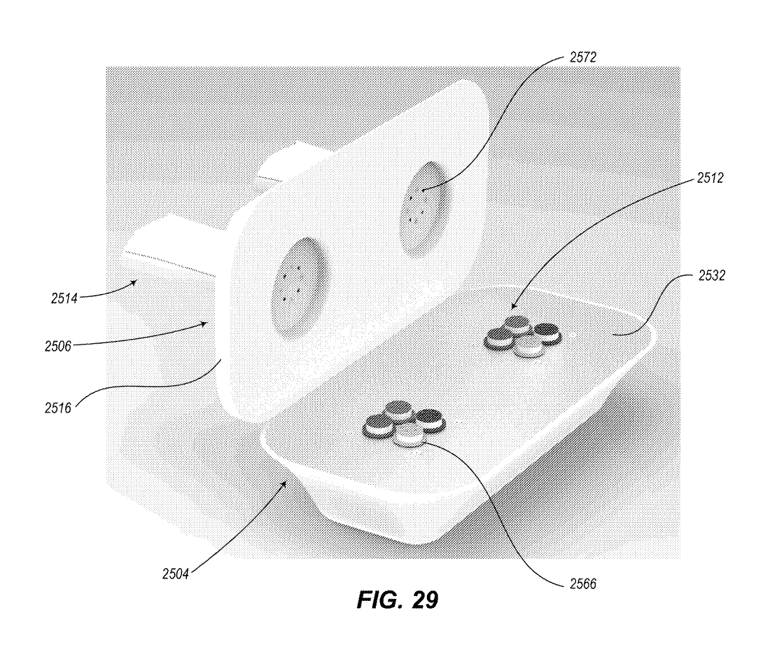

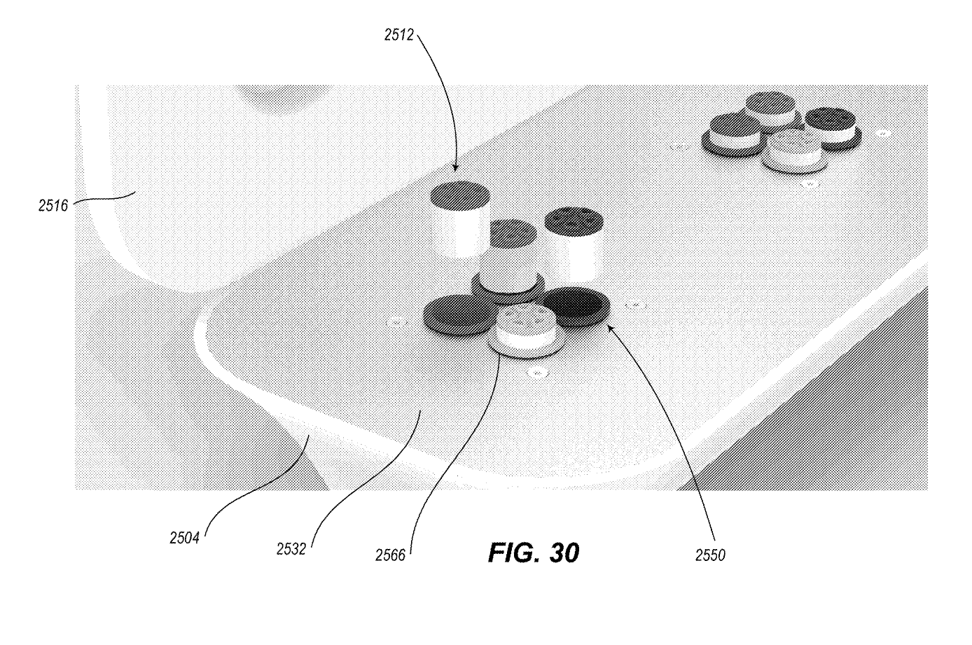

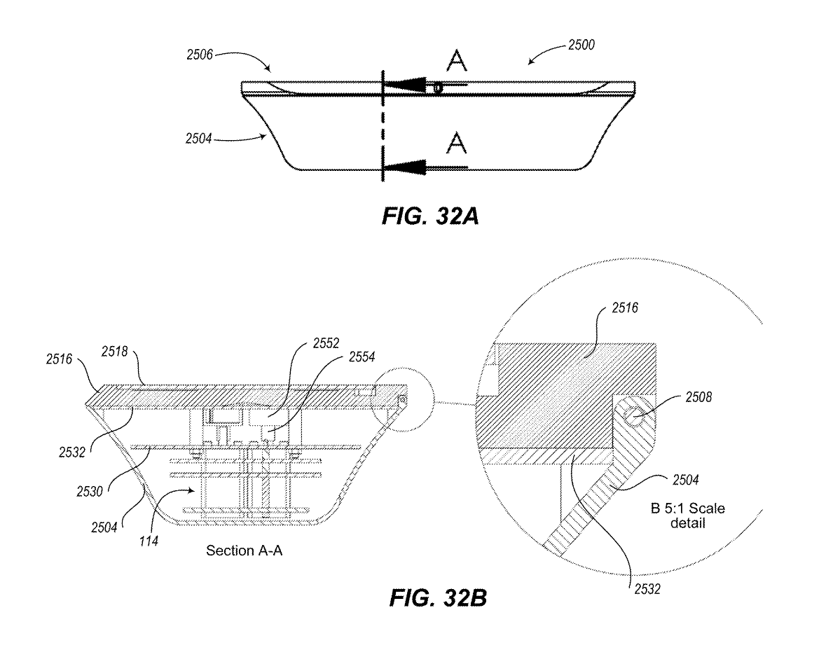

As shown in FIGS. 29, 30, 31A-B, and 32A-B, the lid 2506 of the scent dispensing device 2500 pivots relative to the base 2504 to provide access to the eight scent cartridge receiving portions 2550. During use, a user may selectively rotate the lid 2506 from a closed position shown in FIGS. 32A and 32B wherein the lid is substantially adjacent the top shelf 2532 of the base 2504, to an open position shown in FIGS. 31A and 31B wherein the lid is spaced apart from the top frame. When the lid 2506 is in the open position, the user can selectively position scent cartridges 2512 within the scent cartridge receiving portions 2550 of the scent dispensing device 2500. When the lid 2506 is in the closed position with scent cartridges 2512 positioned within the scent cartridge receiving portions 2550, apertures 2572 (FIG. 29) of the top shelf 2518 of the lid 2506 act as inlet ports to the scent mixing chambers 130 of the scent emitters 2514 (see FIGS. 5A and 5B), which are coupled to the top surface of the top shelf.

FIGS. 33A and 33B illustrate the scent emitter 2514. The scent emitter 2514 is similar in many respects to the scent emitter 118 of FIGS. 2A-5B discussed above, so much of the discussion of the scent emitter 118 applies to the scent emitter 2514. In this embodiment, the scent emitter 2514 includes a light source 2574 (e.g., a light emitting diode (LED)). The light source 2574 may be controlled by electronics disposed on one of the printed circuit boards 2528 to provide various notifications for a user of the scent dispensing device 2500. For example, the light source 2574 may be operatively coupled to the microcontroller 1702 of FIG. 17A. In some embodiments, the light source 2574 may be caused to illuminate when the scent dispensing device 2500 receives a scent message (e.g., from a mobile device). As another example, the light source 2574 may be used to notify a user of status indications for the scent dispensing device 2500. Examples of status indications may include, but are not limited to, power status, battery status, error status, message status, and the like.

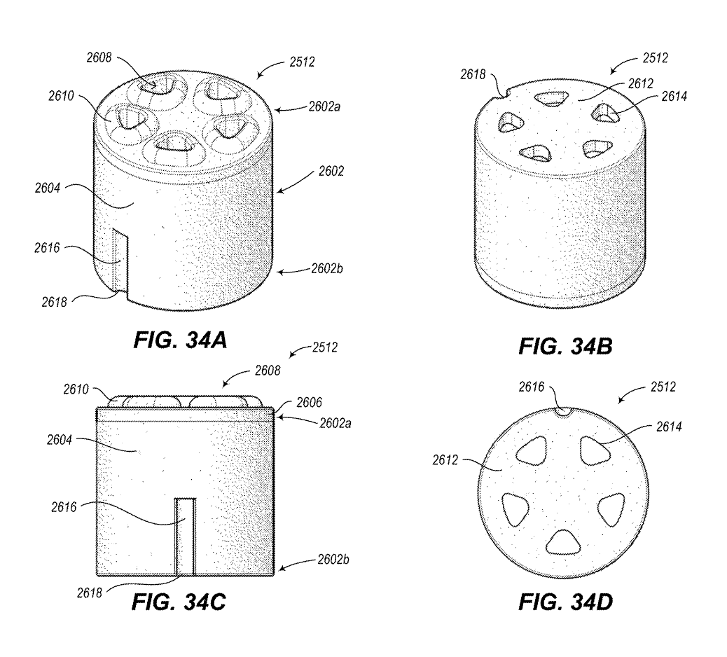

A scent cartridge 2512 according to one illustrated embodiment will now be described with reference to FIGS. 34A-34D. The scent cartridge 2512 is similar in many respects to the scent cartridge 900 discussed above with reference to FIGS. 9A-9D, so much of the discussion regarding the scent cartridge 900 also applies to the scent cartridge 2512.

The scent cartridge 2512 includes a housing 2602 having a first end 2602a and a second end 2602b. A cylindrical outer wall 2604 defines a peripheral perimeter of the housing 2602. A first end portion 2606 is disposed at the first end 2602a of the scent cartridge 2512. The first end portion 2606 includes five wedge-shaped apertures 2608. A raised lip 2610 extends outwardly from the first end portion 2606 around the periphery of each of the apertures 2608. A second end portion 2612 is disposed at the second end 2602b of the scent cartridge 2512. As best shown in FIG. 34B, the second end portion 2612 includes five wedge-shaped apertures 2614. Each of the apertures 2608 formed in the first end portion 2606 is aligned with a corresponding aperture 2614 formed in the second end portion 2612 to define five scent cartridge chambers inside the housing 2602.

The scent cartridge 2512 may have virtually any desired number of scent cartridge chambers. One or more of the chambers may be filled with a scent medium. The scent medium may be a non-liquid substance such as aromatic particles, a scented powder, which may be in a packed powder form, a scented wax, an air filter, or an unscented material. One or more of the chambers may be empty.

A recess or keyway 2616 is disposed on the cylindrical outer wall 2604 of the housing 2602 of the scent cartridge 2512. The keyway 2616 is sized and shaped to slidably receive the key 2568 of the scent cartridge receiving portion 2550 when the scent cartridge 2512 is installed in the scent dispensing device 2500. In the illustrated embodiment, the keyway 2616 extends upward along the cylindrical outer wall 2604 from the second end 2602b to a mid-portion of the outer wall. In other embodiments, the keyway 2616 may be other sizes or shapes so long as its functionality is preserved.

To install the scent cartridge 2512 in the scent dispensing device 2500, the user aligns a bottom portion 2618 of the keyway 2616 of the scent cartridge 2512 with a top portion 2620 of the key 2568 of the body portion 2552 of the scent cartridge receiving portion 2550. The user then moves the scent cartridge 2512 downward relative to the body portion 2552 so that the lower portion of the scent cartridge is disposed within the cylindrical wall 2560 of the body portion 2552 and the key 2568 slidably engages the keyway 2616. When the scent cartridge 2512 is installed within the body portion 2552 as shown in FIGS. 31B and 35B, the second end 2602b of the scent cartridge 2512 abuts the top surface of the base portion 2558 of the body portion 2552 of the scent cartridge receiving portion 2550, and the five wedge-shaped apertures 2562 of the base portion 2558 are aligned with the five wedge-shaped apertures 2614 of the second end portion 2612 of the scent cartridge 2512. Thus air may flow freely though the five wedge-shaped apertures 2562 of the base portion 2558 into the five scent chambers of the scent cartridge 2512. The key 2568 and keyway 2616 operate to permit axial movement between the scent cartridge 2512 and the scent cartridge receiving portion 2550 to allow the scent cartridge to be selectively removable, while restricting rotational movement to ensure that rotation of the scent cartridge receiving portion causes rotation of the scent cartridge.

It should be appreciated that the key 2568 of the body portion 2552 of the scent cartridge receiving portion 2550 and the keyway 2616 of the scent cartridge 2512 may have various sizes and shapes to achieve the desired functionality. Further, in some embodiments, multiple keys and/or keyways may be used. In some embodiments, the scent cartridge 2512 includes one or more keys and the body portion 2552 includes one or more keyways.

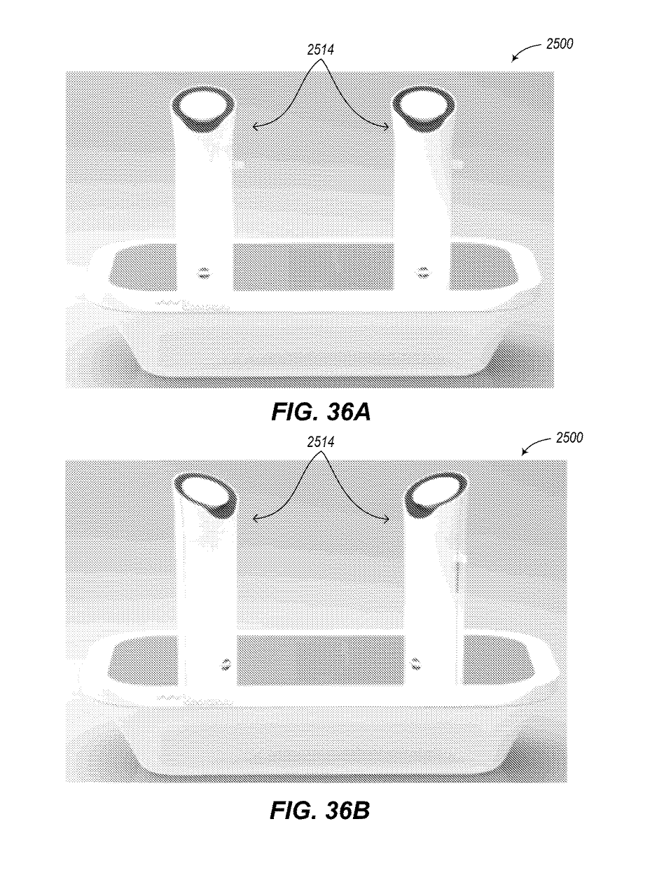

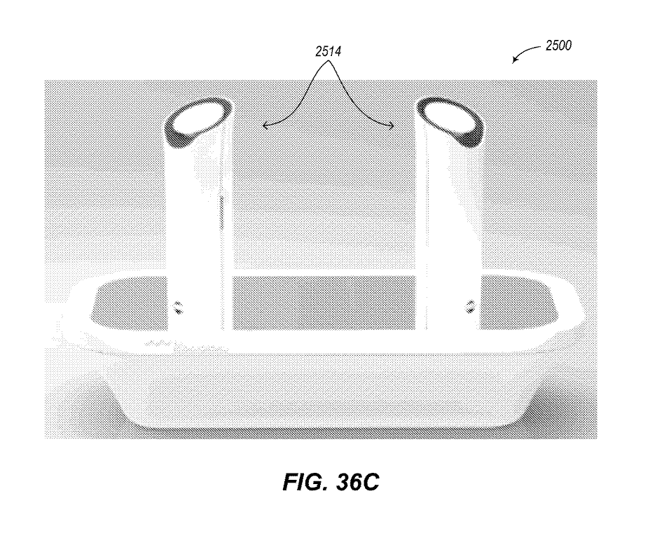

FIGS. 36A-36C illustrate an embodiment of the scent dispensing device 2500 of FIG. 25 in which the two scent emitters 2514 are rotatable about a vertical axis. FIG. 36A shows the scent dispensing device 2500 when the scent emitters 2514 are both facing directly forward. FIG. 36B shows the scent dispensing device 2500 when the scent emitters 2514 are each rotated inward by 45 degrees. FIG. 36C shows the scent dispensing device 2500 when the scent emitters 2514 are each rotated outward by 45 degrees. In some embodiments, the scent emitters 2514 may be rotated manually by a user. In other embodiments, a motor or other mechanical control system may be provided to rotate the scent emitters 2514 automatically. In some embodiments, the scent emitters 2514 may rotate more or less than 45 degrees inward and outward.

A user may position the scent emitters 2514 in the rotated outward position (FIG. 36C) when, for example, two users will be using the scent dispensing device 2500. By dispensing two scents outward from the scent dispensing device 2500, the two users may not need to be positioned as close to each other as would be required if the scent emitters 2514 were facing directly forward. Thus, two users may be positioned at a comfortable distance from each other while each user experiences one or more scents. Additionally, by directing the scent emitters 2514 outward, scents emitted from each of the scent emitters 2514 are separated, allowing two users to experience different scents simultaneously.

A user may position the scent emitters 2514 in the rotated inward position (FIG. 36B) when, for example, a single user will be using the scent dispensing device 2500. In this configuration, the user can simultaneously experience scents emitted from the two scent emitters 2514. This allows the scent dispensing device 2500 to generate scents simultaneously and/or sequentially using any or all of the eight scent cartridges 2512 installed within the scent dispensing device 2500, rather than using only four scent cartridges associated with each of the scent emitters 2514.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments. For example, instead of starting operation of the fan at 2304, the scent dispensing device 1602 may start operation of the fan after the one or more scent cartridges are oriented at 2310. Additionally, instead of stopping operation of the fan at 2320, the scent dispensing device 1602 may stop operation of the fan before returning each of the one or more scent cartridges to the default position at 2318.

In addition, the scent dispensing device 1602 may include a display device and a user input device (e.g., a touch screen) that enables a user to specify scents and/or durations of scents to be dispensed by the scent dispensing device 1602 (e.g., without receiving any message from the first mobile device 1604). The microcontroller of the scent dispensing device 1602 may store instructions that cause the scent dispensing device 1602 to perform the method shown in FIG. 21. Additionally, the microcontroller of the scent dispensing device 1602 may store instructions that cause the scent dispensing device 1602 to display a selectable icon that, when selected, causes the microcontroller to display a user interface that enables a user to specify scents to be dispensed by the scent dispensing device 1602. For example, the user interface may include a plurality of selectable icons, wherein each selectable icon includes an identifier of the scent medium 1234 included in one of the chambers 1232 of one of the scent cartridges loaded in the scent dispensing device 1602. In response to one or more of the icons being selected, the microcontroller causes a corresponding scent or scent combination to be dispensed for a predetermined or a user-specified amount of time.

U.S. provisional patent application Ser. No. 61/891,328 filed Oct. 15, 2013, and U.S. provisional patent application Ser. No. 61/944,862 filed Feb. 26, 2014 are incorporated herein by reference, in their entirety.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of operating a scent dispensing device, the method comprising:
starting operation of an air mover to move air;
following the starting operation of the air mover, orienting via a first motor a first scent cartridge in a first orientation in which a respective outlet of a first one of a plurality of chambers of the first scent cartridge is fluidly communicatively coupled with a first port of the scent dispensing device;
determining via at least one controller a direction of movement and an amount of movement in the determined direction of movement from the first orientation to orient the first scent cartridge in a second orientation in which a respective outlet of a second one of the plurality of chambers is fluidly communicatively coupled with the first port of the scent dispensing device; and
orienting via the first motor the first scent cartridge in the second orientation in which the respective outlet of the second one of the plurality of chambers is fluidly communicatively coupled with the first port of the scent dispensing device.

2. The method of claim 1 wherein determining a direction of movement includes determining which of a first rotational direction and a second rotational direction provides a least amount of movement to move the first scent cartridge from the first orientation to the second orientation, the second rotational direction opposite the first rotational direction.

3. The method of claim 2 wherein determining an amount of movement in the determined direction of movement includes determining a number of steps by a stepper motor in the first rotational direction that will orient the first scent cartridge in the second orientation from the first orientation.

4. The method of claim 1 wherein determining an amount of movement in the determined direction of movement includes determining a number of steps by a stepper motor in the first rotational direction that will orient the first scent cartridge in the second orientation from the first orientation.

5. The method of claim 1 wherein orienting the first scent cartridge in the second orientation includes moving the first scent cartridge from the first orientation in the determined direction of movement by the determined amount of movement.

6. The method of claim 1 wherein the first direction is a first rotational direction, and orienting the first scent cartridge in the second orientation includes driving a stepper motor to move the first scent cartridge in the first rotational direction by a determined first number of steps of the stepper motor.

7. The method of claim 1, further comprising:
in response to a first indication, orienting via the first motor the first scent cartridge in a default orientation.

8. The method of claim 7 wherein orienting the first scent cartridge in the default orientation includes orienting the first scent cartridge such that none of the plurality of chambers which contain scent media are fluidly communicatively coupled with the first port of the scent dispensing device.

9. The method of claim 7 wherein orienting the first scent cartridge in the default orientation includes orienting the first scent cartridge such that a third one of the plurality of chambers which is empty is fluidly communicatively coupled with the first port of the scent dispensing device.

10. The method of claim 7 wherein orienting the first scent cartridge in the default orientation includes orienting the first scent cartridge such that a third one of the plurality of chambers in which an air filter is located is fluidly communicatively coupled with the first port of the scent dispensing device.

11. The method of claim 7, further comprising:
detecting a low available power condition of a power source via the at least one controller, and providing the first indication in response to the detection of the low available power condition.

12. The method of claim 7, further comprising:
detecting a turn off input via the at least one controller, and providing the first indication in response to the detection of the turn off input.

13. The method of claim 7, further comprising:
stopping the operation of the air mover after orienting the first scent cartridge in the default orientation in response to the first indication.

14. The method of claim 13, further comprising:
continuously operating the air mover to move air between the starting operation of the air mover and the stopping of the operation of the air mover in response to the first indication.

15. The method of claim 13, further comprising:
determining a direction of movement and an amount of movement in the determined direction of movement from the second orientation to orient a third one of the plurality of chambers of the first scent cartridge with the first port of the scent dispensing device via the at least one controller; and
orienting via the first motor the first scent cartridge in a third orientation in which a respective outlet of the second one of the plurality of chambers is fluidly communicatively coupled with the first port of the scent dispensing device; and
continuously operating the air mover to move air between the starting operation of the air mover and the stopping of the operation of the air mover in response to the first indication.

16. The method of claim 13, further comprising:
orienting via the first motor the first scent cartridge in the default orientation between orienting the first scent cartridge in the second orientation and orienting the first scent cartridge in a third orientation.

17. The method of claim 7, further comprising:
determining a sequence of orientations of the first scent cartridge based at least in part on a scent specification via the at least one controller, the sequence of orientations including at least the first and the second orientations.

18. The method of claim 17, further comprising;
determining via the at least one controller a direction of movement and an amount of movement in the determined direction of movement from the default orientation to orient the first scent cartridge in the first orientation; and
orienting via the first motor the first scent cartridge in the first orientation in which the respective outlet of the first one of the plurality of chambers is fluidly communicatively coupled with the first port of the scent dispensing device before orienting the first scent cartridge in the second orientation.

19. The method of claim 17, further comprising:
wirelessly receiving the scent specification via a radio prior to determining the sequence of orientations of the first scent cartridge.

20. A scent dispensing device comprising:
an air mover that moves air;
a first motor that orients a first scent cartridge in a first orientation in which a respective outlet of a first one of a plurality of chambers of the first scent cartridge is fluidly communicatively coupled with a first port of the scent dispensing device after operation of the air mover has been started; and
at least one controller that determines a direction of movement and an amount of movement in the determined direction of movement from the first orientation to orient the first scent cartridge in a second orientation in which a respective outlet of a second one of the plurality of chambers is fluidly communicatively coupled with the first port of the scent dispensing device, and that causes the first motor to orient the first scent cartridge in the second orientation in which the respective outlet of the second one of the plurality of chambers is fluidly communicatively coupled with the first port of the scent dispensing device.

21. The device of claim 20 wherein the at least one controller determines which of a first rotational direction and a second rotational direction provides a least amount of movement to move the first scent cartridge from the first orientation to the second orientation, the second rotational direction opposite the first rotational direction.

22. The device of claim 21 wherein the at least one controller determines a number of steps by a stepper motor in the first rotational direction that will orient the first scent cartridge in the second orientation from the first orientation.

23. The device of claim 20 wherein the at least one controller determines a number of steps by a stepper motor in the first rotational direction that will orient the first scent cartridge in the second orientation from the first orientation.

24. The device of claim 20 wherein the at least one controller causes the first motor to move the first scent cartridge from the first orientation in the determined direction of movement by the determined amount of movement.

25. The device of claim 20 wherein the first direction is a first rotational direction, the first motor is a stepper motor, and the at least one controller causes the first motor to move the first scent cartridge in the first rotational direction by a determined first number of steps.

26. The device of claim 20 wherein the at least one controller causes the first motor to orient the first scent cartridge in a default orientation in response to a first indication.

27. The device of claim 26 wherein the at least one controller causes the first motor to orient the first scent cartridge in the default orientation such that none of the plurality of chambers which contain scent media are fluidly communicatively coupled with the first port of the scent dispensing device.

28. The device of claim 26 wherein the at least one controller causes the first motor to orient the first scent cartridge in the default orientation such that a third one of the plurality of chambers which is empty is fluidly communicatively coupled with the first port of the scent dispensing device.

29. The device of claim 26 wherein the at least one controller causes the first motor to orient the first scent cartridge in the default orientation such that a third one of the plurality of chambers in which an air filter is located is fluidly communicatively coupled with the first port of the scent dispensing device.

30. The device of claim 26 wherein the at least one controller detects a low available power condition of a power source and provides the first indication in response to a detection of the low available power condition.

31. The device of claim 26 wherein the at least one controller detects a turn off input and provides the first indication in response to a detection of the turn off input.

32. The device of claim 26 wherein the at least one controller stops the operation of the air mover after orienting the first scent cartridge in the default orientation in response to the first indication.

33. The device of claim 32 wherein the at least one controller causes the air mover to continuously operate to move air between starting operation of the air mover and stopping operation of the air mover in response to the first indication.

34. The device of claim 32 wherein the at least one controller:
determines a direction of movement and an amount of movement in the determined direction of movement from the second orientation to orient a third one of the plurality of chambers of the first scent cartridge with the first port of the scent dispensing device via the at least one controller, and
causes the first motor to orient the first scent cartridge in a third orientation in which a respective outlet of the second one of the plurality of chambers is fluidly communicatively coupled with the first port of the scent dispensing device, and
causes the air mover to continuously operate to move air between starting operation of the air mover and stopping operation of the air mover in response to the first indication.

35. The device of claim 32 wherein the at least one controller causes the first motor to orient the first scent cartridge in the default orientation between orienting the first scent cartridge in the second orientation and orienting the first scent cartridge in a third orientation.

36. The device of claim 26 wherein the at least one controller determines a sequence of orientations of the first scent cartridge based at least in part on a scent specification, the sequence of orientations including at least the first and the second orientations.

37. The device of claim 36 wherein the at least one controller:
determines a direction of movement and an amount of movement in the determined direction of movement from the default orientation to orient the first scent cartridge in the first orientation; and
causes the first motor to orient the first scent cartridge in the first orientation in which the respective outlet of the first one of the plurality of chambers is fluidly communicatively coupled with the first port of the scent dispensing device before orienting the first scent cartridge in the second orientation.

38. The device of claim 36, further comprising:
a communications module that wirelessly receives the scent specification via a radio, wherein the communications module receives the scent specification before the at least one controller determines the sequence of orientations of the first scent cartridge.

39. A method of operating a scent dispensing device, the method comprising:
receiving a first scent cartridge having a plurality of chambers at least some of the chambers having respective scent media therein;
with the first scent cartridge in a default orientation, starting operation of an air mover to move air with respect to the first scent cartridge;
following the starting operation of the air mover, orienting via a first motor the first scent cartridge from the default orientation to a first orientation in which a respective outlet of a first one of the plurality of chambers of the first scent cartridge is fluidly communicatively coupled with a first port of the scent dispensing device; and
in response to an indication of a stop condition, returning the first scent cartridge to the default orientation.

40. The method of claim 39 wherein returning the first scent cartridge to the default orientation includes orienting the first scent cartridge such that none of the plurality of chambers which contain scent media are fluidly communicatively coupled with the first port of the scent dispensing device.

41. The method of claim 39 wherein returning the first scent cartridge to the default orientation includes orienting the first scent cartridge such that one of the plurality of chambers which is empty is fluidly communicatively coupled with the first port of the scent dispensing device.

42. The method of claim 39 wherein returning the first scent cartridge to the default orientation includes orienting the first scent cartridge such that one of the plurality of chambers in which an air filter is located is fluidly communicatively coupled with the first port of the scent dispensing device.

43. The method of claim 39, further comprising:
detecting a low available power condition of a power source via at least one controller, providing the indication of the stop condition in response to the detection of the low available power condition.

44. The method of claim 39, further comprising:
detecting a turn off input via at least one controller, and providing the indication of the stop condition in response to the detection of the turn off input.

45. The method of claim 39, further comprising:
stopping the operation of the air mover after orienting the first scent cartridge in the default orientation in response to the indication of the stop condition.

46. The method of claim 45, further comprising:
continuously operating the air mover to move air between the starting operation of the air mover and the stopping of the operation of the air mover in response to the indication of the stop condition.

47. A scent dispensing device comprising:
a first scent cartridge having a plurality of chambers at least some of the chambers having respective scent media therein;
an air mover that moves air with respect to the first scent cartridge;
a first motor that changes an orientation of the first scent cartridge; and
at least one controller, wherein the at least one controller:
starts operation of air mover with the first scent cartridge in a default orientation,
following the starting operation of the air mover, causes the first motor to orient the first scent cartridge from the default orientation to a first orientation in which a respective outlet of a first one of the plurality of chambers of the first scent cartridge is fluidly communicatively coupled with a first port of the scent dispensing device, and
in response to an indication of a stop condition, causes the first motor to return the first scent cartridge to the default orientation via the first motor.

48. The device of claim 47 wherein the at least one controller causes the first motor to return the first scent cartridge to the default orientation such that none of the plurality of chambers which contain scent media are fluidly communicatively coupled with the first port of the scent dispensing device.

49. The device of claim 47 wherein the at least one controller causes the first motor to return the first scent cartridge to the default orientation such that one of the plurality of chambers which is empty is fluidly communicatively coupled with the first port of the scent dispensing device.

50. The device of claim 47 wherein the at least one controller causes the first motor to return the first scent cartridge to the default orientation such that one of the plurality of chambers in which an air filter is located is fluidly communicatively coupled with the first port of the scent dispensing device.

51. The device of claim 47 wherein the at least one controller detects a low available power condition of a power source, and provides the indication of the stop condition in response to a detection of the low available power condition.

52. The device of claim 47 wherein the at least one controller detects a turn off input, and provides the indication of the stop condition in response to the detection of the turn off input.

53. The device of claim 47 wherein the at least one controller causes the operation of the air mover to stop after causing the first motor to orient the first scent cartridge in the default orientation in response to the indication of the stop condition.

54. The device of claim 53 wherein the at least one controller causes the air mover to continuously operate to move air between the starting operation of the air mover and the stopping of the operation of the air mover in response to the indication of the stop condition.

55. A method of operation in a scent sequence specification device, the method comprising:
causing a presentation of a scent sequence specification user interface, the scent sequence specification user interface including a scent palette of a set of user selectable scent icons, each of the user selectable scent icons corresponding to a respective scent, the scent sequence specification user interface further including an array of scent specification fields which are selectively populatable via selections of the user selectable scent icons from the scent palette; and
receiving inputs via the scent sequence specification user interface; and
generating a scent specification file based at least in part on the inputs received via the scent sequence specification user interface.

56. The method of claim 55 wherein causing the presentation of the scent sequence specification user interface includes causing a presentation of the scent sequence specification user interface including two or more categories of scents.

57. The method of claim 56 wherein causing the presentation of the scent sequence specification user interface includes causing a presentation of the scent sequence specification user interface including user selectable icons for two or more scents in each of the categories of scents.

58. The method of claim 56 wherein causing the presentation of the scent sequence specification user interface includes causing a presentation of the scent sequence specification user interface including, in each of the categories of scents, two or more user selectable icons for two or more scents arranged spatially sequentially with respect to one another in the respective category.

59. The method of claim 56 wherein causing the presentation of the scent sequence specification user interface includes causing a presentation of the scent sequence specification user interface including, in each of the categories of scents, two or more user selectable icons for two or more scents arranged spatially sequentially in columns with respect to one another in the respective category.

60. The method of claim 55 wherein causing the presentation of the scent sequence specification user interface includes causing a presentation of a first array of user selectable scent icons each of which represents a respective scent of a first category of scent, and at least a second array of user selectable scent icons each of which represents a respective scent of a second category of scent, the second category of scent different from the first category of scent.

61. The method of claim 55 wherein causing the presentation of the scent sequence specification user interface includes causing a presentation of at least two of the following:
a first array of user selectable scent icons each of which represents a respective scent of a coffee category of scent,
a second array of user selectable scent icons each of which represents a respective scent of a chocolate category of scent,
a third array of user selectable scent icons each of which represents a respective scent of a caramel category of scent, or
a fourth array of user selectable scent icons each of which represents a respective scent of a nut category of scent.

62. The method of claim 55 wherein causing the presentation of the scent sequence specification user interface includes causing a presentation of the scent sequence specification user interface including an array of scent specification fields where a temporal sequence is represented by distribution of the scent specifications fields along a first axis of the array of scent specification fields.

63. The method of claim 55 wherein causing the presentation of the scent sequence specification user interface includes causing a presentation of the scent sequence specification user interface including a two-dimensional array of scent specification fields having a first axis and second axis perpendicular to the first axis, where a distribution of the scent specifications fields along the first axis of the array represents a position in a temporal sequence and a distribution of the scent specification field along the second axis of the array represents one or more scents to be dispensed at a respective position in the temporal sequence.

64. The method of claim 55 wherein causing the presentation of the scent sequence specification user interface includes causing a presentation of the scent sequence specification user interface including a name field, and further comprising:
logically associating a name entered in the name field with the scent specification file.

65. The method of claim 55 wherein causing the presentation of the scent sequence specification user interface includes causing a presentation of the scent sequence specification user interface including a recipient address field and a submit user selectable icon; and further comprising:
in response to selection of the submit user selectable icon, causing the scent specification file to be transmitted to an address specified via the recipient address field.

66. The method of claim 55 wherein causing a presentation of a scent sequence specification user interface includes providing a set of processor executable instructions to a processor-based device.

67. The method of claim 55 wherein causing a presentation of a scent sequence specification user interface includes wirelessly providing at least one markup language file to a mobile communications device via a server computer.

68. The method of claim 55 wherein causing a presentation of a scent sequence specification user interface includes providing a set of Web services via a server computer.

69. A scent sequence specification device comprising:
at least one processor; and
a computer-readable storage medium storing instructions that, when executed by the at least one processor, cause the scent sequence specification device to:
present a scent sequence specification user interface, the scent sequence specification user interface including a scent palette of a set of user selectable scent icons, each of the user selectable scent icons corresponding to a respective scent, the scent sequence specification user interface further including an array of scent specification fields which are selectively populatable via selections of the user selectable scent icons from the scent palette; and
receiving inputs via the scent sequence specification user interface; and
generating a scent specification file based at least in part on the inputs received via the scent sequence specification user interface.

70. The device of claim 69 wherein the scent sequence specification user interface includes two or more categories of scents.

71. The device of claim 70 wherein the scent sequence specification user interface includes user selectable icons for two or more scents in each of the categories of scents.

72. The device of claim 70 wherein the scent sequence specification user interface includes, in each of the categories of scents, two or more user selectable icons for two or more scents arranged spatially sequentially with respect to one another in the respective category.

73. The device of claim 70 wherein the scent sequence specification user interface includes, in each of the categories of scents, two or more user selectable icons for two or more scents arranged spatially sequentially in columns with respect to one another in the respective category.

74. The device of claim 69 wherein the scent sequence specification user interface includes a first array of user selectable scent icons each of which represents a respective scent of a first category of scent, and at least a second array of user selectable scent icons each of which represents a respective scent of a second category of scent, the second category of scent different from the first category of scent.

75. The device of claim 69 wherein the scent sequence specification user interface includes at least two of the following:
a first array of user selectable scent icons each of which represents a respective scent of a coffee category of scent,
a second array of user selectable scent icons each of which represents a respective scent of a chocolate category of scent,
a third array of user selectable scent icons each of which represents a respective scent of a caramel category of scent, or
a fourth array of user selectable scent icons each of which represents a respective scent of a nut category of scent.

76. The device of claim 69 wherein the scent sequence specification user interface includes an array of scent specification fields where a temporal sequence is represented by distribution of the scent specifications fields along a first axis of the array of scent specification fields.

77. The device of claim 69 wherein the scent sequence specification user interface includes a two-dimensional array of scent specification fields having a first axis and second axis perpendicular to the first axis, where a distribution of the scent specifications fields along the first axis of the array represents a position in a temporal sequence and a distribution of the scent specification field along the second axis of the array represents one or more scents to be dispensed at a respective position in the temporal sequence.

78. The device of claim 69 wherein the scent sequence specification user interface includes a name field, the at least one processor logically associates a name entered in the name field with the scent specification file.

79. The device of claim 69 wherein the scent sequence specification user interface includes a recipient address field and submit user selectable icon and, in response to selection of the submit user selectable icon, the at least one processor causes the scent specification file to be transmitted to an address specified via the recipient address field.

80. The device of claim 69 wherein the at least one processor presents the scent sequence specification user interface by providing a set of processor executable instructions to a processor-based device.

81. The device of claim 69 wherein the at least one processor presents the scent sequence specification user interface by wirelessly providing at least one markup language file to a mobile communications device.

82. The device of claim 69 wherein the at least one processor presents the scent sequence specification user interface by providing a set of Web services.

83. A consumable scent cartridge, comprising:
a housing having at least one wall defining a peripheral perimeter, a first end, a second end opposed across a length of the housing from the first end, and a plurality of chambers in the housing annularly arrayed about a rotational axis thereof, at least two of the chambers extending through the first and the second ends, each of the chambers separated and distinct from one another; and
a plurality of consumable scent media, a respective one of the consumable scent media received in a respective one of each of at least two of the chambers.

84. The consumable scent cartridge of claim 83 wherein at least one of the chambers is empty.

85. The consumable scent cartridge of claim 83 wherein at least one of the chambers contains a medium that is unscented.

86. The consumable scent cartridge of claim 83 wherein at least one of the chambers contains an air filter medium that removes scent from air that passes therethrough.

87. The consumable scent cartridge of claim 83 wherein each of the chambers has an aperture at least proximate the first end, the apertures each having a respective dimensional area, and the first end includes at least one portion spaced between two of the apertures, the at least one portion having a dimensional area equal to the respective dimensional area of the apertures.

88. The consumable scent cartridge of claim 83 wherein each of the consumable scent media is in a non-liquid form.

89. The consumable scent cartridge of claim 83 wherein each consumable scent medium is in a powder form.

90. The consumable scent cartridge of claim 83 wherein each consumable scent medium is in a packed powder form.

91. The consumable scent cartridge of claim 83 wherein each consumable scent medium is in a wax form.

92. The consumable scent cartridge of claim 83 wherein, for each of the chambers, there is a gap between the consumable scent medium and an inner surface of at least one wall of the respective chamber, the gap extending along the length of the housing.

93. The consumable scent cartridge of claim 83 wherein, for each of the chambers, there is a gap between the consumable scent medium and an inner surface of the at least one wall that defines the peripheral perimeter of the housing.

94. The consumable scent cartridge of claim 83 wherein the housing has a circular peripheral perimeter and each chamber is a wedge-shaped segment of a circular profile of the circular peripheral perimeter.

95. The consumable scent cartridge of claim 94 wherein, for each of the chambers, there is a gap between the consumable scent medium and an inner surface of the at least one wall that defines the peripheral perimeter of the housing, and each consumable scent medium is adjacent at least one other wall that forms the respective chamber.

96. The consumable scent cartridge of claim 94, further comprising:
at least one selective membrane at each of the first and the second ends, which at least one selective membrane retains consumable scent media in a solid form and passes air and scented air, and wherein, for each of the chambers, there is a gap between the consumable scent medium and all walls that form the respective chamber, the consumable scent media in contact with the respective selective membranes.

97. The consumable scent cartridge of claim 96 wherein the at least one selective membrane has a nominal pore size small enough to retain consumable scent media in a solid form and large enough to allow passage of air and scented air.

98. The consumable scent cartridge of claim 83 wherein each of the consumable scent media have a respective scent, the scents of a respective scent medium received in each of the at least two of the chambers being different from one another.

99. The consumable scent cartridge of claim 83 wherein the housing includes an annular bearing surface at the second end of the housing, the annular bearing surface disposed about the axis of rotation.

100. The consumable scent cartridge of claim 83, further comprising:
a first and a second hermetically sealed membrane removably located respectively across the first and the second ends of the housing.

101. A consumable scent cartridge, comprising:
a housing having at least one wall defining a peripheral perimeter, a first end, a second end opposed across a length of the housing from the first end, and a plurality of chambers, at least two of the chambers extending through the first and the second ends, each of the chambers separated and distinct from one another; and
a plurality of consumable scent media, each of the consumable scent media received in respective ones of each of at least two of the chambers.

102. The consumable scent cartridge of claim 101 wherein each consumable scent medium is in a solid form.

103. The consumable scent cartridge of claim 101 wherein each consumable scent medium is in a powder form.

104. The consumable scent cartridge of claim 101 wherein each consumable scent medium is in a packed powder form.

105. The consumable scent cartridge of claim 101 wherein each consumable scent medium is in a wax form.

106. The consumable scent cartridge of claim 101, further comprising:
at least one selective membrane at each of the first and the second ends, the selective membranes which retain the consumable scent media in the respective chambers while passing air into and scented air out of the respective chambers.

107. The consumable scent cartridge of claim 106 wherein, for each of the chambers, there is a gap between the consumable scent medium and all walls that form the respective chamber, the consumable scent media in contact with the respective selective membranes.

108. The consumable scent cartridge of claim 101 wherein the housing has a circular peripheral perimeter and each chamber is a wedge-shaped segment of a circular profile of the circular peripheral perimeter.

109. The consumable scent cartridge of claim 101 wherein each of the consumable scent media have a respective scent, the scents of a respective scent medium received in each of the at least two of the chambers being different from one another.

110. The consumable scent cartridge of claim 101, further comprising:
a first and a second hermetically sealed membrane removably located respectively across the first and the second ends of the housing to hermetically seal the chambers at least prior to use.

111. A consumable scent cartridge, comprising:
a housing having a first end, a second end opposed across a length of the housing from the first end, and a plurality of chambers in the housing, each of the chambers separated and distinct from one another and having an aperture at least proximate the first end of the housing;
a plurality of consumable scent media in a particulate form, a respective one of the consumable scent medium located in a respective one of each of at least two of the chambers; and
at least one first end selective membrane at least proximate the first end of the housing and positioned across at least the apertures of the at least two of the chambers in which the consumable scent medium is located, the at least one first end selective membrane which selectively passes scented air while not passing the consumable scent media in the particulate form.

112. The consumable scent cartridge of claim 111 wherein each of the chambers additionally has an aperture at least proximate the second end of the housing, further comprising:
at least one second end selective membrane at least proximate the second end of the housing and positioned across at least the apertures of the at least two of the chambers in which the consumable scent medium is located, the at least one second end selective membrane which selectively passes scented air while not passing the consumable scent media in the particulate form.

113. The consumable scent cartridge of claim 111 wherein each of the chambers forms a fluid flow path within the housing, extending through the first and the second ends of the housing.

114. The consumable scent cartridge of claim 111 wherein each consumable scent medium is in a powder form.

115. The consumable scent cartridge of claim 111 wherein each consumable scent medium is in a packed powder form.

116. The consumable scent cartridge of claim 101 wherein each consumable scent medium is in a wax form.

117. The consumable scent cartridge of claim 111 wherein, for each of the chambers, there is a gap between the consumable scent medium and all walls that form the respective chamber, the consumable scent media in contact with at least one of the respective selective membranes.

118. The consumable scent cartridge of claim 111, further comprising:
a first and a second hermetically sealed membrane removably located respectively across the first and the second ends of the housing to hermetically seal the chambers at least prior to use.

119. The consumable scent cartridge of claim 118 wherein the first and the second hermetically sealed membranes are foil membranes with a pressure sensitive adhesive about at least a peripheral portion thereof.

120. A consumable scent kit, comprising:
a first consumable scent cartridge having a plurality of chambers, two or more of the chambers containing consumable scent media; and
at least a second consumable scent cartridge having a plurality of chambers, two or more of the chambers containing consumable scent media in a non-liquid form, at least some of the consumable scent media of the second consumable scent cartridge having a scent that is a different scent from that of at least some of the consumable scent media of the first consumable scent cartridge.

121. The consumable scent kit of claim 120 wherein at least one of the chambers is empty.

122. The consumable scent kit of claim 120 wherein at least one of the chambers contains a medium that is unscented.

123. The consumable scent kit of claim 120 wherein at least one of the chambers contains a scent neutralizing medium.

124. The consumable scent kit of claim 120 wherein at least one of the chambers contains an air filter medium that removes scent from air that passes therethrough.

125. The consumable scent kit of claim 120 wherein each consumable scent medium is in a non-liquid form.

126. The consumable scent kit of claim 120 wherein each consumable scent medium is a solid.

127. The consumable scent kit of claim 120 wherein each consumable scent medium is a reservoir of particulate.

128. The consumable scent kit of claim 127 wherein the reservoir of particulate is a powder.

129. The consumable scent kit of claim 127 wherein the reservoir of particulate is a packed powder.

130. The consumable scent kit of claim 101 wherein each consumable scent medium is in a wax form.

131. The consumable scent kit of claim 120, further comprising:
a first pair of hermetically sealed membranes removably located respectively across a first end and a second end of the first consumable scent cartridge to hermetically seal the chambers thereof at least prior to use; and
a second pair of hermetically sealed membranes removably located respectively across a first end and a second end of the second consumable scent cartridge to hermetically seal the chambers thereof at least prior to use.

132. The consumable scent kit of claim 131 wherein the hermetically sealed membranes are foil membranes each with a respective tab and with a pressure sensitive adhesive about at least a peripheral portion thereof, the pressure sensitive which allows resealing of the chambers after use.

133. The consumable scent kit of claim 120, further comprising:
a first hermetically sealed pouch which contains at least the first consumable scent cartridge prior to use.

134. The consumable scent kit of claim 133 wherein the first hermetically sealed pouch also contains at least the second consumable scent cartridge prior to use.

135. The consumable scent kit of claim 133, further comprising:
a second hermetically sealed pouch which contains at least the second consumable scent cartridge prior to use, the first and the second hermetically sealed pouches packaged together prior to use.

136. The consumable scent kit of claim 120, further comprising:
a blister pack including a backing substrate and a blister substrate carried by the backing substrate, the blister substrate having a plurality of blisters to receive respective ones of the first and at the second first consumable scent cartridges therein prior to use.

137. The consumable scent kit of claim 120 wherein the consumable scent media contained in each of the chambers of the first consumable scent cartridge which contain consumable scent media have a different scent from one another.

138. The consumable scent kit of claim 120 wherein the consumable scent media contained in each of the chambers of the first consumable scent cartridge which contain consumable scent media have a same scent as each other.

139. A consumable scent cartridge, comprising:
a housing having a first end, a second end opposed across a length of the housing from the first end, and a plurality of chambers in the housing, each of the chambers separated and distinct from one another and having an aperture at least proximate the first end of the housing; and
a plurality of consumable scent media, a respective one of the consumable scent media located in a respective one of each of at least two of the chambers, with at least fifty percent of a total surface area of the consumable scent medium exposed to an airflow through the chamber.

140. The consumable scent cartridge of claim 139 wherein each consumable scent media emits a scent at least during use and is in a non-liquid form.

141. The consumable scent cartridge of claim 140 wherein each consumable scent media is in a powder form.

142. The consumable scent cartridge of claim 140 wherein each consumable scent media is in a packed powder form.

143. The consumable scent cartridge of claim 139 wherein, for each of the chambers, there is a gap between the consumable scent medium and an inner surface of at least one wall of the respective chamber, the gap extending along the length of the housing.

144. The consumable scent cartridge of claim 139 wherein, for each of the chambers, there is a gap between the consumable scent medium and an inner surface of at least one wall that defines a peripheral perimeter of the housing.

145. The consumable scent cartridge of claim 139 wherein the housing has a circular peripheral perimeter and each chamber is a wedge-shaped segment of a circular profile of the circular peripheral perimeter.

146. The consumable scent cartridge of claim 145 wherein, for each of the chambers, there is a gap between each consumable scent media medium and an inner surface of at least one wall that defines a peripheral perimeter of the housing, and each consumable scent medium is adjacent at least one other wall that forms the respective chamber.

147. The consumable scent cartridge of claim 145 further comprising:
at least one selective membrane at each of the first and the second ends, and wherein, for each of the chambers, there is a gap between the consumable scent medium and all walls that form the respective chamber, the consumable scent media in contact with the respective selective membranes.

148. The consumable scent cartridge of claim 139 wherein the at least one selective membrane at each of the first and the second ends includes at least one first end selective membrane at least proximate the first end of the housing and positioned across at least the apertures of the at least two of the chambers in which the consumable scent medium is located, the at least one first end selective membrane which selectively passes air or scented air while not passing the consumable scent media in the particulate form.

149. The consumable scent cartridge of claim 148, further comprising:
a first and a second hermetically sealed membrane removably located respectively across the first and the second ends of the housing.

150. The consumable scent cartridge of claim 148, further comprising an asymmetric coupler to detachably couple the cartridge to an asymmetric portion of a drive shaft.

151. A scent dispensing device to dispense a scent from a removable scent cartridge, the removable scent cartridge including body having a plurality of chambers each having a respective outlet, the body of the removable scent cartridge including an exterior surface having a keyway, the scent dispensing device comprising:
an air mover that moves air;
a first motor comprising an output shaft;
a coupling sleeve coupled to the output shaft of the first motor and rotatable thereby, the coupling sleeve defining a volume shaped and sized to receive at least a portion of the exterior surface of the body of the removable scent cartridge, the coupling sleeve including a protruding key that slidably engages with the keyway of the removable scent cartridge to releasably interconnect the coupling sleeve with the removable scent cartridge such that the output shaft of the first motor transmits rotary motion to the coupling sleeve which in turn transmits rotary motion to the removable scent cartridge, wherein the first motor orients the removable scent cartridge in a first orientation in which a respective outlet of a first one of the plurality of chambers of the removable scent cartridge is fluidly communicatively coupled with a first port of the scent dispensing device after operation of the air mover has been started; and
a controller that determines a direction of movement and an amount of movement in the determined direction of movement from the first orientation to orient the removable scent cartridge in a second orientation in which a respective outlet of a second one of the plurality of chambers is fluidly communicatively coupled with the first port of the scent dispensing device, and that causes the first motor to orient the removable scent cartridge in the second orientation in which the respective outlet of the second one of the plurality of chambers is fluidly communicatively coupled with the first port of the scent dispensing device.

152. The device of claim 151 wherein the coupling sleeve comprises a base portion and a cylindrical wall extending upwardly therefrom, and the protruding key is disposed on an interior surface of the cylindrical wall.

153. The device of claim 152 wherein the base portion of the coupling sleeve comprises a plurality of apertures each aligned with a respective outlet of the plurality of chambers of the removable scent cartridge.

154. The device of claim 151 wherein the coupling sleeve comprises a motor shaft coupling portion comprising an aperture shaped and sized to receive at least a portion of the output shaft of the first motor.

155. The device of claim 151, further comprising:
a light source operatively coupled to the controller, wherein the controller causes the light source to emit light upon the occurrence of one or more conditions.

156. The device of claim 151 wherein the one or more conditions comprises the scent dispensing device receiving a scent specification that specifies at least one scent.

157. The device of claim 151 wherein the controller determines which of a first rotational direction and a second rotational direction provides a least amount of movement to move the removable scent cartridge from the first orientation to the second orientation, the second rotational direction opposite the first rotational direction.

158. The device of claim 157 wherein the controller determines a number of steps by a stepper motor in the first rotational direction that will orient the removable scent cartridge in the second orientation from the first orientation.

159. The device of claim 151 wherein the controller determines a number of steps by a stepper motor in the first rotational direction that will orient the removable scent cartridge in the second orientation from the first orientation.

160. The device of claim 151 wherein the controller causes the first motor to move the removable scent cartridge from the first orientation in the determined direction of movement by the determined amount of movement.

161. The device of claim 151 wherein the first direction is a first rotational direction, the first motor is a stepper motor, and the controller causes the first motor to move the removable scent cartridge in the first rotational direction by a determined first number of steps.

162. The device of claim 151 wherein the controller causes the first motor to orient the removable scent cartridge in a default orientation in response to a first indication.

163. The device of claim 162 wherein the controller causes the first motor to orient the removable scent cartridge in the default orientation such that none of the plurality of chambers which contain scent media are fluidly communicatively coupled with the first port of the scent dispensing device.

164. The device of claim 162 wherein the controller causes the first motor to orient the removable scent cartridge in the default orientation such that a third one of the plurality of chambers which is empty is fluidly communicatively coupled with the first port of the scent dispensing device.

165. The device of claim 162 wherein the controller causes the first motor to orient the removable scent cartridge in the default orientation such that a third one of the plurality of chambers in which an air filter is located is fluidly communicatively coupled with the first port of the scent dispensing device.

166. The device of claim 162 wherein the controller detects a low available power condition of a power source and provides the first indication in response to a detection of the low available power condition.

167. The device of claim 162 wherein the controller detects a turn off input and provides the first indication in response to a detection of the turn off input.

168. The device of claim 162 wherein the controller stops the operation of the air mover after orienting the removable scent cartridge in the default orientation in response to the first indication.

169. The device of claim 168 wherein the controller causes the air mover to continuously operate to move air between starting operation of the air mover and stopping operation of the air mover in response to the first indication.

170. The device of claim 168 wherein the controller:
determines a direction of movement and an amount of movement in the determined direction of movement from the second orientation to orient a third one of the plurality of chambers of the removable scent cartridge with the first port of the scent dispensing device via the controller, and
causes the first motor to orient the removable scent cartridge in a third orientation in which a respective outlet of the second one of the plurality of chambers is fluidly communicatively coupled with the first port of the scent dispensing device, and
causes the air mover to continuously operate to move air between starting operation of the air mover and stopping operation of the air mover in response to the first indication.

171. The device of claim 168 wherein the controller causes the first motor to orient the removable scent cartridge in the default orientation between orienting the removable scent cartridge in the second orientation and orienting the removable scent cartridge in a third orientation.

172. The device of claim 162 wherein the controller determines a sequence of orientations of the removable scent cartridge based at least in part on a scent specification, the sequence of orientations including at least the first and the second orientations.

173. The device of claim 172 wherein the controller:
determines a direction of movement and an amount of movement in the determined direction of movement from the default orientation to orient the removable scent cartridge in the first orientation; and
causes the first motor to orient the removable scent cartridge in the first orientation in which the respective outlet of the first one of the plurality of chambers is fluidly communicatively coupled with the first port of the scent dispensing device before orienting the removable scent cartridge in the second orientation.

174. The device of claim 172, further comprising:
a communications module that wirelessly receives the scent specification via a radio, wherein the communications module receives the scent specification before the controller determines the sequence of orientations of the removable scent cartridge.

175. The device of claim 174, further comprising:
a light source operatively coupled to the controller, wherein the controller causes the light source to emit light when the communications module receives the scent specification.

176. A scent dispensing device,
a body having a top surface;
a plurality of spaced apart scent dispensing subunits coupled to the body, each of the plurality of scent dispensing subunits comprising:
an air mover that moves air;
a motor that orients a scent cartridge in a first orientation in which a respective outlet of a first one of a plurality of chambers of the scent cartridge is fluidly communicatively coupled with a port of the scent dispensing subunit after operation of the air mover has been started;
an elongate scent channel fluidly communicatively coupled with the port of the scent dispensing subunit, the elongate scent channel attached to and extending upwardly from the top surface of the body and being selectively rotatable about a vertical axis; and
a controller that determines a direction of movement and an amount of movement in the determined direction of movement from the first orientation to orient the scent cartridge in a second orientation in which a respective outlet of a second one of the plurality of chambers is fluidly communicatively coupled with the port of the scent dispensing subunit, and that causes the motor to orient the scent cartridge in the second orientation in which the respective outlet of the second one of the plurality of chambers is fluidly communicatively coupled with the port of the scent dispensing subunit.

177. The scent dispensing device of claim 176 wherein the body of the scent dispensing device includes a lower body portion, and the top surface is rotatably coupled to the lower body portion.

178. The scent dispensing device of claim 176 wherein the plurality of spaced apart scent dispensing units comprises two spaced apart scent dispensing units, and each of the elongate scent channels of the respective scent dispensing units is rotatable over a range of at least 90 degrees.

179. A scent dispensing device to dispense a scent from a removable scent cartridge, the removable scent cartridge including body having a plurality of chambers each having a respective outlet, the body of the removable scent cartridge including an exterior surface having a keyway, the scent dispensing device comprising:
a scent channel comprising an air mover that moves air, the scent channel further comprising a first port;
a first motor comprising an output shaft;
a coupling sleeve coupled to the output shaft of the first motor and rotatable thereby, the coupling sleeve defining a volume shaped and sized to receive at least a portion of the exterior surface of the body of the removable scent cartridge, the coupling sleeve including a protruding key that slidably engages with the keyway of the removable scent cartridge to releasably interconnect the coupling sleeve with the removable scent cartridge such that the output shaft of the first motor transmits rotary motion to the coupling sleeve which in turn transmits rotary motion to the removable scent cartridge, wherein the first motor orients the removable scent cartridge in a first orientation in which a respective outlet of a first one of the plurality of chambers of the removable scent cartridge is fluidly communicatively coupled with the first port of the scent channel after operation of the air mover has been started; and
a controller that determines a direction of movement and an amount of movement in the determined direction of movement from the first orientation to orient the removable scent cartridge in a second orientation in which a respective outlet of a second one of the plurality of chambers is fluidly communicatively coupled with the first port of the scent channel, and that causes the first motor to orient the removable scent cartridge in the second orientation in which the respective outlet of the second one of the plurality of chambers is fluidly communicatively coupled with the first port of the scent channel.

180. The device of claim 179, further comprising:
a base portion that houses the first motor, the coupling sleeve and the controller; and
a lid portion, wherein the scent channel is coupled to the lid portion, and the lid portion is rotatably coupled to the base portion.

181. The device of claim 179 wherein the scent channel comprises a light source operatively coupled to the controller, and the controller causes the light source to emit light upon the occurrence of one or more conditions.

182. A scent dispensing device to dispense a scent from a removable scent cartridge, the removable scent cartridge including body having a plurality of chambers each having a respective outlet, the body of the removable scent cartridge including an exterior surface having a first coupling portion, the scent dispensing device comprising:
an air mover that moves air;
a first motor comprising an output shaft;
a coupling sleeve coupled to the output shaft of the first motor and rotatable thereby, the coupling sleeve defining a volume shaped and sized to receive at least a portion of the exterior surface of the body of the removable scent cartridge, the coupling sleeve including a second coupling portion that engages the first coupling portion of the removable scent cartridge to releasably interconnect the coupling sleeve with the removable scent cartridge such that the output shaft of the first motor transmits rotary motion to the coupling sleeve which in turn transmits rotary motion to the removable scent cartridge, wherein the first motor orients the removable scent cartridge in a first orientation in which a respective outlet of a first one of the plurality of chambers of the removable scent cartridge is fluidly communicatively coupled with a first port of the scent dispensing device after operation of the air mover has been started; and
a controller that determines a direction of movement and an amount of movement in the determined direction of movement from the first orientation to orient the removable scent cartridge in a second orientation in which a respective outlet of a second one of the plurality of chambers is fluidly communicatively coupled with the first port of the scent dispensing device, and that causes the first motor to orient the removable scent cartridge in the second orientation in which the respective outlet of the second one of the plurality of chambers is fluidly communicatively coupled with the first port of the scent dispensing device.

183. A consumable scent cartridge, comprising:
a housing having at least one wall defining a peripheral perimeter, a first end, a second end opposed across a length of the housing from the first end, and a plurality of chambers in the housing arrayed about a rotational axis thereof, at least two of the chambers extending through the first and the second ends, each of the chambers separated and distinct from one another, the wall comprising one of a key and a keyway that mates with the other of a key and a keyway of a coupling portion of a scent dispensing device to define a keyed joint, the keyed joint permitting axial movement between the housing of the consumable scent cartridge and the coupling portion of the scent dispensing device and restricting rotational movement therebetween about the rotational axis of housing; and
a plurality of consumable scent media, a respective one of the consumable scent media received in a respective one of each of at least two of the chambers.

184. The consumable scent cartridge of claim 183 wherein the wall comprises a keyway extending upwardly along the wall from the second end.

185. A consumable scent cartridge, comprising:
a housing having at least one wall defining a peripheral perimeter, a first end, a second end opposed across a length of the housing from the first end, and a plurality of chambers in the housing arrayed about a rotational axis thereof, at least two of the chambers extending through the first and the second ends, each of the chambers separated and distinct from one another, the wall comprising a recess that mates with a protrusion of a coupling portion of a scent dispensing device to define a keyed joint, the keyed joint permitting axial movement between the housing of the consumable scent cartridge and the coupling portion of the scent dispensing device and restricting rotational movement therebetween about the rotational axis of housing; and
a plurality of consumable scent media, a respective one of the consumable scent media received in a respective one of each of at least two of the chambers.

186. A consumable scent cartridge, comprising:
a housing having at least one wall defining a peripheral perimeter, a first end, a second end opposed across a length of the housing from the first end, and a plurality of chambers in the housing arrayed about a rotational axis thereof, at least two of the chambers extending through the first and the second ends, each of the chambers separated and distinct from one another, the wall comprising exterior coupling portion that mates with interior coupling portion of a coupling sleeve of a scent dispensing device to define a keyed joint, the keyed joint permitting axial movement between the housing of the consumable scent cartridge and the coupling portion of the scent dispensing device and restricting rotational movement therebetween about the rotational axis of housing; and
a plurality of consumable scent media, a respective one of the consumable scent media received in a respective one of each of at least two of the chambers.
